(19) United States
(12) Patent Application Publication
(10) Pub. No.: US 2009/0270431 A1
(43) Pub. Date: Oct. 29, 2009

(54) Cyclopentenol Nucleoside Compounds Intermediates for their Synthesis and Methods of Treating Viral Infections

(75) Inventors: David C.K. Chu, Statham, GA (US); Jong Hyun Cho, Athens, GA (US); Hyo-Joong Kim, Gainsville, FL (US)

Correspondence Address: COLEMAN SUDOL SAPONE, P.C., 714 COLORADO AVENUE, BRIDGE PORT, CT, 06605-1601, US

(73) Assignee: THE UNIVERSITY OF GEORGIA RESEARCH FOUNDATION, Athens, GA (US)

(21) Appl. No.: 12/083,571

(22) PCT Filed: Oct. 19, 2006
(86) PCT No.: PCT/US06/40773

§ 371 Date: Jun. 23, 2008

Related U.S. Application Data
(60) Provisional application No. 60/728,107, filed on Oct. 19, 2005.

(51) Int. Cl. A61K 31/519; C07D 249/04; A61K 31/4192; A61K 31/437; C07D 471/04; C07D 487/04; A61P 31/12

(52) U.S. Cl. 514/265.1; 548/255; 514/359; 514/303; 546/118; 544/280

(57) ABSTRACT

The present invention relates to compounds according to the structure (I), Where B is formula (Ia), formula (Ib) or formula (Ic); A is H, OR² or halogen (F, Cl, Br, I, preferably F or Br, more preferably F); A′ is H, OR2 or halogen (F, Cl, Br, I, preferably F or Br, more preferably F); A″ is H or OR¹, with the proviso that when A′ is OR, A is H; and when A is OR², A′ is H; X is C—R³ or N; Y is C—R³ or N; preferably X or Y is N and X and Y are not both simultaneously N; R³ is H or C₁-C₃ alkyl; D is H or NHR²; E is absent or H; G is O or NHR²; J is N or C—R⁴; K is N or C—H; R⁴ is H, halogen (F, Cl, Br, I), CN, —C(═O)NH₂, NH₂, NO₂, —C═C—H (cis or trans) or —C≡C—H; Rₐ is H or CH₃; Each R¹ is independently H, an acyl group, a C₁-C₂₀ alkyl or ether group, a phosphate, diphosphate, triphosphate, phosphodiester group; Each R² is independently H, an acyl group, a C₁-C₂₀ alkyl or ether group; and Pharmaceutically acceptable salts, solvates or polymorphs thereof.

### RELATED APPLICATIONS

This application claims the benefit of priority from provisional application number US60/728,107, filed Oct. 19, 2006, which is incorporated by reference in its entirety herein.

The research which gave rise to the present invention was supported by one or more U.S. Public Health Service Grants. Accordingly, the government retains certain rights in the invention.

### FIELD OF THE INVENTION

The present invention relates to novel nucleoside compounds, intermediate compounds for making certain of these compounds, pharmaceutical compositions comprising these novel compounds, methods of treating viral infections and methods of making compounds according to the present invention.

### BACKGROUND OF THE INVENTION

Neplanocin A (NPA),¹ a carbocyclic nucleosides isolated from Ampullariella regularis, has received a great deal of attention as antiviral or antitumor agents.² Inhibition of S-adenosylhomocysteine hydrolase (AdoHcy-ase) is responsible for the biological activity, which is the key enzyme in the regulation of S-adenosyl-L-methionine (AdoMet)-dependent methylation reactions during mRNA replication cycles.³ NPA is also a substrate for adenosine kinase as well as adenosine deaminase, and exhibits cellular toxicity.⁴

Based on these interesting biological results, significant amounts of synthetic efforts have been directed toward finding more selective analogues⁵. As part of these efforts, a number of 6′-modified analogues, such as (6′R)-6′-C-methylneplanocin A (RMNPA, 2a)^{5e} and 6′-homoneplanocin A (HNPA, 2b)⁵ⁱ have been synthesized (FIG. 1). 3-Deazaneplanocin A (2c)^{5d} and its analogue (2d)^{5a} also showed potent bioactivities against various viruses, including orthopoxyirues. Our group has reported synthetic method for NPA and several NPA analogues as well as their antiviral activities.⁶ Among the modified NPA analogues, cytosine (3a) and fluorocytosine (3b) analogues were found to be active against HIV, West Nile virus and orthopox viruses.⁶

The synthesis of NPA analogues have utilized a chiral cyclopentenol as the key intermediate, starting from optically pure carbohydrates or tartaric acids by various synthetic methods.⁷ Recently, the ring-closing metathesis (RCM) reaction,⁸ one of the most powerful methods for the formation of small-sized rings via C—C double bonds, has been employed for the synthesis of disubstituted cyclopentenols.⁹ Although a few examples used RCM reaction as the key synthetic step for the tri-substituted cyclopentenol derivatives, large amounts of Grubbs catalysts were necessary to complete the reaction¹⁰ and its reaction conditions were difficult to control when Schrock's catalysts were used.¹¹ Herein, we wish to report efficient and practical method for the synthesis of cyclopentenol (+)-12a from D-ribose (4) and its utilization for the synthesis of novel biologically active five-ring heterocyclic NPA analogues (23a-c) as potential antiviral agents.

### BRIEF DESCRIPTION OF THE FIGURES

FIG. 1 shows scheme 1 which sets forth the synthesis of a blocked cyclopentenol synthon for use in the present invention.

FIG. 2 shows scheme 2 which sets forth in the synthesis of triazolo-substituted cyclopentenol compounds according to the present invention.

FIG. 3 shows the scheme 1A synthesis of chloropurine analog 9. The reaction conditions for the scheme include the following conditions and reagents: a) DIAD, Ph₃P, THF, 0° C. then −78° C., rt, 24 h; b) HCl/MeOH, rt, 6 h; c) NH₂NH₂, reflux, 3 h, then Raney-Nickel, rt, 4 h; d) NH₃/MeOH, 150° C., 48 h.

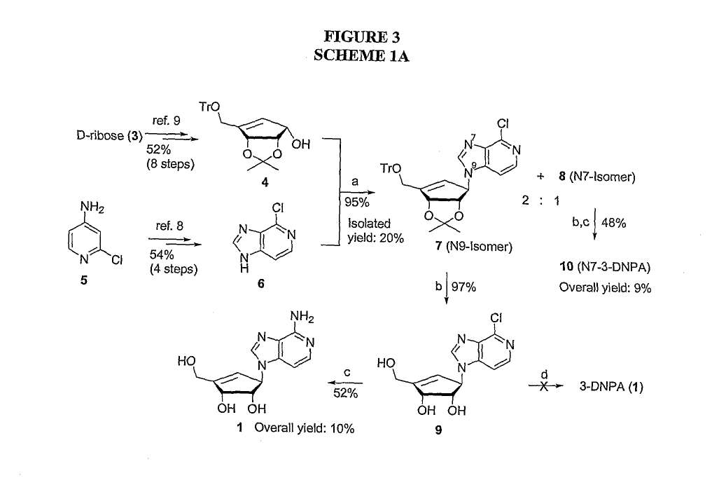

FIG. 4 shows a synthetic scheme for the production of 3-Deazanaplanocin A (1). The reaction conditions for the scheme include the following conditions and reagents: a) 20 wt. % of LiN₃ in water, DMF, [emim]BF₄, 80° C., 8 h; b) H₂ (3 atm), Pd/C, 6N HCl, rt, 36 h; c) i. benzoyl chloride, pyridine, rt, 24 h (13a); ii. (Boc)₂O, DMAP, THF, rt, 24 h (13b); d) i. sat. pyridine, EtOH, reflux, 3 h (14a); ii. 1.0 M NaHCO₃, THF, 0° C. (14b and 14d); iii. NaOMe, THF, rt (14c); e) DIAD, Ph₃P, THF, 0° C.→−78° C., rt, 24 h, f) sat. NH₃/MeOH, 100° C., 12 h in steel bomb; g) HCl/MeOH, rt, 3 h.

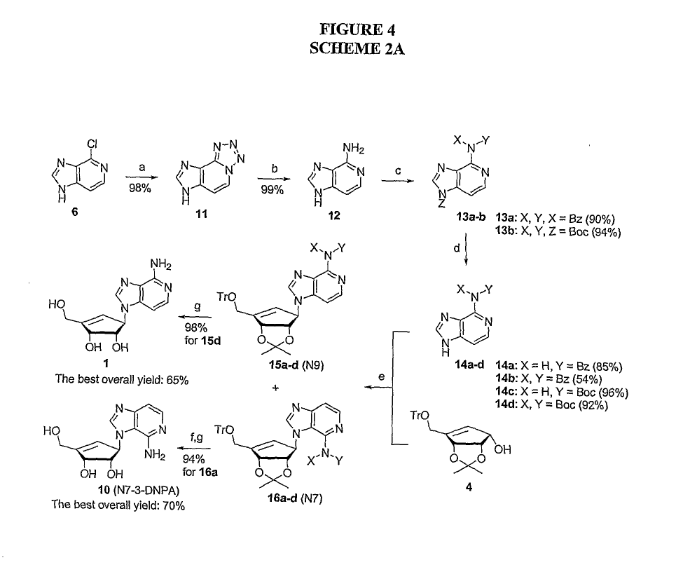

FIG. 5 shows synthetic scheme 1C which relates to the synthesis of 7-deazaadeninecyclopentenol analog. The FIG. 5 reaction condition and reagents: a) 20 wt. % of LiN₃ in water, DMF, [emim]BF₄, 80° C., 8 h, b) H₂ (3 atm), Pd/C, 6N HCl, rt, 36 h; c) i. benzoyl chloride, pyridine, rt, 24 h (13a); ii. (Boc)₂O, DMAP, THF, rt, 24 h (13b); d) i. sat. pyridine, EtOH, reflux, 3 h (14a); ii. 1.0 M NaHCO₃, THF, 0° C. (14b and 14d); iii. NaOMe, THF, rt (14c); e) DIAD, Ph₃P, THF, 0° C.→−78° C., rt, 24 h; f) sat. NH₃/MeOH, 100° C., 12 h in steel bomb; g) HCl/MeOH, rt, 3 h.

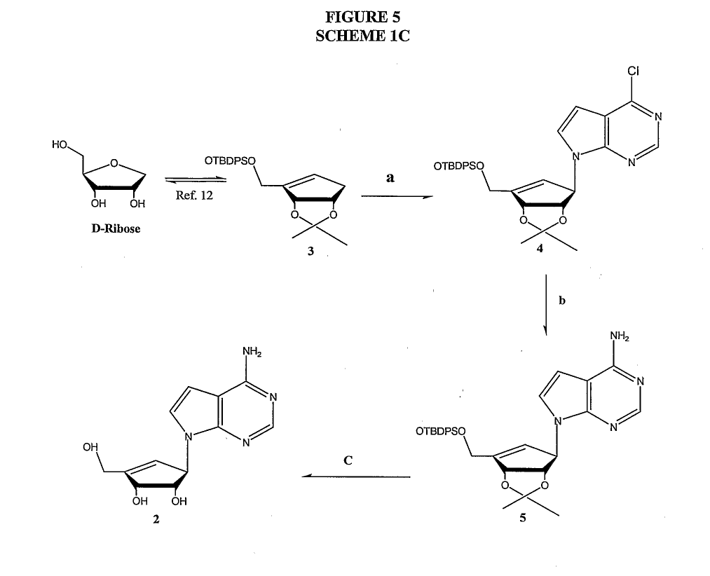

FIG. 6 shows a number of preferred 1,3-deazaadenine derivatives which can be used in the present invention to synthesize relevant cyclopentenol compounds according to the present invention.

### BRIEF DESCRIPTION OF THE INVENTION

The present invention relates to compounds according to the structure I:

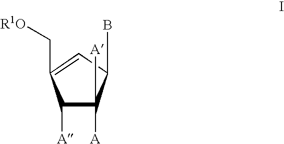

### Where B is

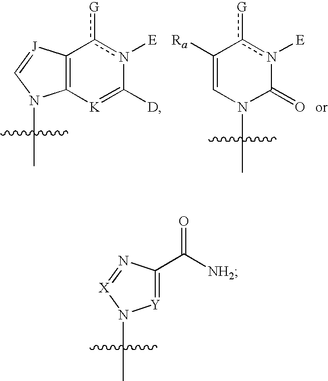

A is H, OR² or halogen (F, Cl, Br, I, preferably F or Br, more preferably F);
A′ is H, OR² or halogen (F, Cl, Br, I, preferably F or Br, more preferably F);

### A″ is H or OR¹,

with the proviso that when A′ is OR², A is H; and when A is OR², A′ is H;

### X is C—R³ or N;

Y is C—R³ or N; preferably X or Y is N and X and Y are not both simultaneously N;
R³ is H or C₁-C₃ alkyl;

### D is H or NHR²;

E is absent (when G is NHR²) or H (when G is O);

### G is O or NHR²;

### J is N or C—R⁴;

### K is N or C—H;

R⁴ is H, halogen (F, Cl, Br, I), CN, —C(═O)NH₂, NH₂, NO₂, —C═C—H (cis or trans) or —C≡C—H;

### Rₐ is H or CH₃;

Each R¹ is independently H, an acyl group, a C₁-C₂₀ alkyl or ether group, a phosphate, diphosphate, triphosphate, phosphodiester group;
Each R² is independently H, an acyl group, a C₁-C₂₀ alkyl or ether group; and
pharmaceutically acceptable salts, solvates or polymorphs thereof.

In certain preferred aspects of the present invention, A is OH, A′ is H and A″ is OH, J is CR⁴, K is N or CH, X is N, Y is CR³, E is absent and G is NHR². In other preferred embodiments, J is N, K is CH and G is O or NHR². In many preferred embodiments, R¹ and R² are both H. In certain preferred embodiments, R⁴ is an acetylenic group. In other preferred embodiments, the preferred compound is

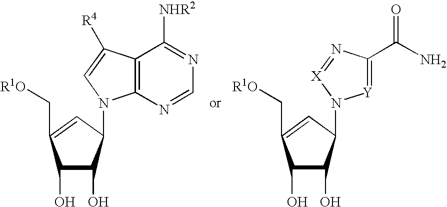

Where R¹, R², R⁴, X and Y are the same as described above. Other preferred compounds may be readily gleaned from the description of the invention which follows.

The present invention also relates to pharmaceutical compositions comprising an effective amount of any one or more of the compounds described above, optionally in combination with a pharmaceutically acceptable carrier, additive or excipient.

Thus, the present application is directed to the treatment of disease states or conditions including viral infections, especially Orthopox virus infections including alastrim, vaccinia, variola (smallpox), cowpox, ectromelia, monkeypox, rabbitpox, severe acute respiratory syndrome virus-associated coronavirus (SARS virus), measles virus (family Paramyxoviridae, genus Morbillivirus), human cytomegalovirus (HCMV), hepatitis B virus (HBV), hepatitis C virus (HCV), vaccinia virus (especially in immunocompromised individuals), Herpes simplex virus I and II (HSV-1 and HSV-2), Varicella-Zoster virus (chicken pox and shingles) (VZV), yellow fever virus, dengue virus, tacaribe virus, Rhinovirus (common cold), adenovirus, influenza A (flu A, including strains H1N1, H3N2 and H3N3), influenza B (flu B), respiratory syncytial virus (RSV), parainfluenza virus (PIV), including drug resistant strains of these viruses, comprising administering to a patient in need thereof an effective amount of a pharmaceutical composition comprising any one or more of the compounds previously described above, optionally in combination with a pharmaceutically acceptable carrier, additive or excipient. A further application or method involves reducing the likelihood of a patient contracting an infection from one or more of the above viruses, comprising administering to a patient at risk of such a virus infection an effective amount of one or more compounds according to the present invention, optionally in combination with a pharmaceutically acceptable carrier, additive or excipient.

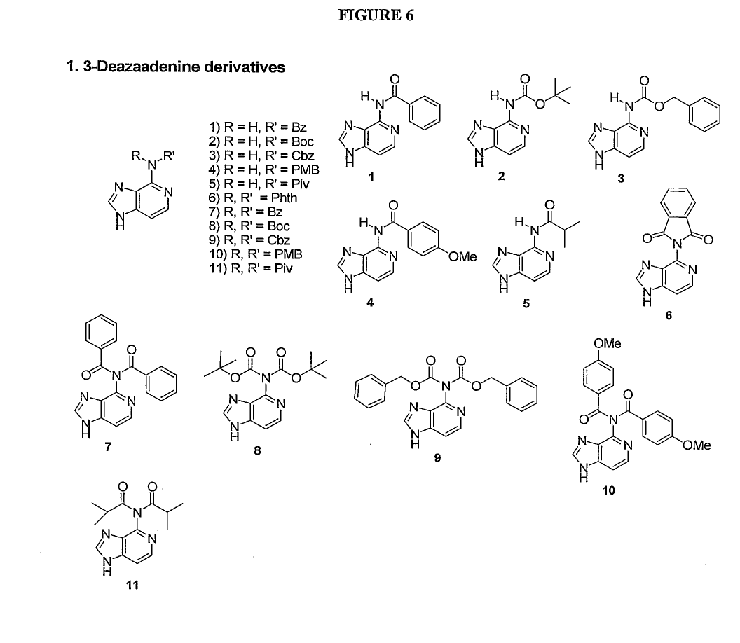

The present invention also relates to compounds according to the structures II or III:

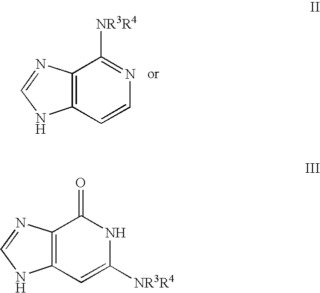

Where R³ and R⁴ are the same or different and are independently H, a COR^{a} group or a COOR^{b} group (preferably R³ and R⁴ are identical), or when R³ and R⁴ are both COR^{a} groups, R³ and R⁴ together with the nitrogen to which they are attached may form a single or multi-ring system having two keto groups alpha to the nitrogen in the single or multi-ring system, with the proviso that both R³ and R⁴ are not simultaneously H;
Each R^{a} is the same or different and is independently a C₁-C₂₅ optionally substituted hydrocarbyl group (preferably each R^{a} is identical);
Each R^{b} is the same or different and is independently a C₁-C₂₅ optionally substituted hydrocarbyl group (preferably each R^{b} is identical); and
salts, solvates and polymorphs thereof. Preferred compounds include those which are provided in FIG. 6 hereof.

In another aspect of the present invention, a method of synthesizing the compound 3-deazaadenine is provided by reacting a compound according to the structure:

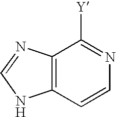

where Y′ is Cl, Br, or I, preferably Cl
With an azide salt (preferably sodium or lithium azide) in the presence of ionic liquid to produce a compound according to the structure

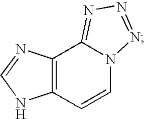

Which is further subjected to hydrogenation conditions (e.g. H₂ Pd/C, HCl, etc.) to convert the azide group to an amino group to produce the compound

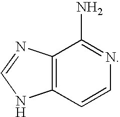

### DETAILED DESCRIPTION OF THE INVENTION

The following terms shall be used to describe the present invention. In instances where a term is not specifically defined herein, the definition given to that term is that which is used within the context of the present invention by those of ordinary skill in the art.

“Patient” refers to an animal, preferably a mammal, even more preferably a human, in need of treatment or therapy to which compounds according to the present invention are administered in order to treat a condition or disease state treatable using compounds according to the present invention.

The term “compound” is used herein to refer to any specific chemical compound disclosed herein. Within its use in context, the term generally refers to a single compound, but in certain instances may also refer to stereoisomers and other positional isomers and/or optical isomers (including racemic mixtures) of disclosed compounds. The compounds of this invention include all stereoisomers where relevant (e.g., cis and trans isomers, such as of vinyl groups) and all optical isomers of the present compounds (eg., R and S enantiomers), as well as racemic, diastereomeric and other mixtures of such isomers, as well as all polymorphs and hydrates of the present compounds, where applicable. Note that a dashed line [symbol] which represents a bond between two atoms in a compound signifies that the bond may be a single bond or a double bond, depending upon the substituents (if any) on the atoms to which the dashed line is attached. By way of example, in exemplary purine compounds according to the invention, where G is an oxygen atom (O), the bond between O and the carbon atom to which it is attached is a double bond and the bond between the carbon to which the oxygen is bonded and the alpha nitrogen is a single bond, and E (which is bonded to the nitrogen atom alpha to the carbon) is H. When G is a NHR² group, then the bond between NHR² and the carbon atom to which it is attached is a single bond and the bond between the carbon to which the nitrogen of NHR² is bonded and the alpha nitrogen is a double bond, and E (which is bonded to the nitrogen atom alpha to the carbon) is non-existent.

“Hydrocarbon” or “hydrocarbyl” refers to any monovalent radical containing carbon and hydrogen, which may be straight, branch-chained or cyclic in nature. Hydrocarbons include linear, branched and cyclic hydrocarbons, including alkyl groups, alkylene groups, saturated and unsaturated hydrocarbon groups, including aromatic groups both substituted and unsubstituted.

“Alkyl” refers to a fully saturated monovalent radical containing carbon and hydrogen, and which may be cyclic, branched or a straight chain. Examples of alkyl groups are methyl, ethyl, n-butyl, n-hexyl, n-heptyl, n-octyl, isopropyl, 2-methylpropyl, cyclopropyl, cyclopropylmethyl, cyclobutyl, cyclopentyl, cyclopentylethyl, cyclohexylethyl and cyclohexyl. Preferred alkyl groups are C₁-C₂₀ alkyl groups. “Alkylene” refers to a fully saturated hydrocarbon which is divalent (may be linear, branched or cyclic) and which is optionally substituted. The term “ether” shall mean a C₁ to C₂₀ ether group, formed from an oxygen and an alkyl group at a position on the sugar moiety of compounds according to the present invention, or alternatively, may also contain at least one oxygen group within the alkyl chain.

The term “acyl” is used throughout the specification to describe a group at the 5′ position of the nucleoside analog (i.e., at the free hydroxyl position in the sugar or cyclopentenoside synthon) which contains a C₁ to C₂₀ linear, branched or cyclic alkyl chain. The acyl group at the 5′ position, in combination with the 5′ hydroxyl group results in an ester, which, after administration, may be cleaved to produce the free nucleoside form of the present invention. Acyl groups according to the present invention are represented by the structure:

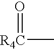

where R₄ is a C₁ to C₂₀ linear, branched or cyclic alkyl group, alkoxyalkyl, aryloxyalkyl, such as phenoxymethyl, aryl, alkoxy, among others. Preferred acyl groups are those where R⁴ is a C₁ to C₁₀ alkyl group. Acyl groups according to the present invention also include, for example, those acyl groups derived from benzoic acid and related acids, 3-chlorobenzoic acid, succinic, capric and caproic, lauric, myristic, palmitic, stearic and oleic groups, among numerous others including mesylate groups. One of ordinary skill in the art will recognize the acyl groups which will have utility in the present invention, either to synthesize the target pharmaceutical compounds or as prodrug forms of the nucleosides according to the present invention.

The term “phosphate ester” or “phosphodiester” is used throughout the specification to describe mono-phosphate groups at the 5′ position of the cyclopentenoside moiety or sugar synthon which are diesterified such that the phosphate group is rendered neutral, i.e., has a neutral charge. Phosphate esters for use in the present invention include those represented by the structures:

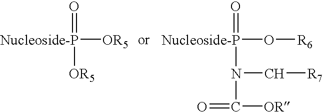

where R₅, R₆ and R″ are selected from a C₁ to C₂₀ linear, branched or cyclic alkyl group, alkoxyalkyl, aryloxyalkyl, such as phenoxymethyl, aryl and alkoxy, among others, and R₇ is a C₁ to C₂₀ linear, branched or cyclic alkyl or acyl group, alkoxyalkyl, aryloxyalkyl, such as phenoxymethyl, aryl and alkoxy, among others. Preferred monophosphate esters for use in prodrug forms according to the present invention are those where R₅ is a C₁ to C₂₀ is a linear or branched chain alkyl group, more preferably a C₁ to C₃ alkyl group.

Other terms used to indicate substituent groups in compounds according to the present invention are as conventionally used in the art.

“Aryl” or “aromatic” refers to a substituted or unsubstituted monovalent aromatic radical having a single ring (e.g., benzene) or multiple condensed. rings (e.g., naphthyl, anthracenyl, phenanthryl), which may be optionally substituted and can be can be bound to the compound according to the present invention at any position on the ring(s) (preferably, for example, benzyl).

The term “cyclic” shall refer to an optionally substituted carbocyclic or heterocyclic group, preferably a 3-7-membered ring, preferably a 5- or 6-membered ring. A heterocyclic ring or group shall be a ring containing between 3 and 7 atoms of which up to four of those atoms are other than carbon and are selected from nitrogen, sulfur and oxygen. Carbocyclic and heterocyclic rings according to the present invention may be unsaturated or saturated.

The term “effective amount” refers to the amount of a selected compound which is effective within the context of its use or administration. In the case of therapeutic methods according to the present invention, the precise amount required will vary depending upon the particular compound selected, the age and weight of the subject, route of administration, and so forth, but may be easily determined by routine experimentation. Compounds according to the present invention may be used to treat or prevent viral infections (by for example, inhibition the growth, replication or elaboration of the virus).

The term “substituted” shall mean substituted at a carbon (or nitrogen) position with, in context, hydroxyl, carboxyl, cyano (C≡N), nitro (NO₂), halogen (preferably, 1, 2 or 3 halogens, especially on an allyl, especially a methyl group such as a trifluoromethyl), thiol, alkyl group (preferably, C₁-C₆, more preferably, C₁-C₃), alkoxy group (preferably, C₁-C₆ alkyl or aryl, including phenyl), ester (preferably, C₁-C₆ alkyl or aryl) including alkylene ester (such that attachment is on the alkylene group, rather than at the ester function which is preferably substituted with a C₁-C₆ alkyl or aryl group), thioether (preferably, C₁-C₆ alkyl or aryl), thioester (preferably, C₁-C₆ alkyl or aryl), (preferably, C₁-C₆ alkyl or aryl), halogen (F, Cl, Br, I), nitro or amine (including a five- or six-membered cyclic alkylene amine, including a C₁-C₆ alkyl amine or C₁-C₆ dialkyl amine), alkanol (preferably, C₁-C₆ alkyl or aryl), or alkanoic acid (preferably, C₁-C₆ alkyl or aryl). Preferably, the term “substituted” shall mean within its context of use alkyl, alkoxy, halogen, hydroxyl, carboxylic acid, nitro and amine (including mono- or di-alkyl substituted amines). The term unsubstituted shall mean substituted with one or more H atoms.

The term “virus” shall be used to describe all types of viruses, the growth or replication of which may be inhibited or disease states of which may be treated using one or more methods according to the present invention. Viruses which may be treated preferably according to the present invention include, for example, the Orthopox viruses, including alastrim, vaccinia, variola (smallpox), cowpox, ectromelia, monkeypox, rabbitpox and severe acute respiratory syndrome virus-associated coronavirus (SARS virus), among others, including measles virus (family Paramyxoviridae, genus Morbillivirus), human cytomegalovirus (HCMV), hepatitis B virus (HBV), hepatitis C virus (HCV), vaccinia virus (especially in immunocompromised individuals), Herpes simplex virus I and II (HSV-1 and HSV-2), Varicella-Zoster virus (chicken pox and shingles) (VZV), yellow fever virus, dengue virus, tacaribe virus, Rhinovirus (common cold), adenovirus, influenza A (flu A, including strains H1N1, H3N2 and H3N3), influenza B (flu B), respiratory syncytial virus (RSV) and parainfluenza virus (PIV), including drug resistant forms of each of these viruses.

The term “ionic liquid” refers to conditions which are used to introduce an azide onto the 6-position of 3-deazaadenine according to the present invention. Ionic liquid is an imidazolium complex generally in a polar aprotic solvent such as DMF or DMA (at about 1:5 to about 1:20 v:v, preferably about 1:10 v:v) identified by the following:

[emim]BF4; 1-Ethyl-3-methylimidazolium tetrafluoroborate;

[emim]Cl; 1-Ethyl-3-methylimidazolium chloride;

[emim]OTf; 1-Ethyl-3-methylimidazolium triflate;

[bmim]BF4; 1-n-Butyl-3-methylimidazolium tetrafluoroborate;

[bmim]Cl; 1-n-Butyl-3-methylimidazoliumchloride;

[bmim]PF6; 1-n-Butyl-3-methylimidazolium hexafluorophosphate;

[bmim]OTf; 1-n-Butyl-3-methylimidazoliumtriflate; and

[bmim]SbF6; 1-n-Butyl-3-methylimidazolium hexafluoroantimonate.

In the present invention, introduction of an azido moiety on the 1 and 6-positions of 3-deazapurine to form the corresponding tricyclic tetraazole compound by displacement of a halogen at the 6-position with a salt of azide (preferably sodium azide, potassium azide or lithium azide) in ionic liquid as described above, followed by isolating the resulting tricyclic tetrazole compound and then hydrogenating the tetrazole group to convert to a 6-amino group (3-deazaadenine) in an unexpectedly high yield (at least 85+%, preferably at least 90+%, even more preferably at least about 95%, and most preferably at least about 98% or virtually quantitative yield), or alternatively, in a single pot reaction, converting the 6-halo-3-deazapurine compound to the tetrazole group in ionic liquid and then subjecting the tricyclic tetrazole intermediate to hydrogenation conditions to afford 3-deazaadenine, also in very high yield as described above.

Alternatively, the 6-halo-3-deazapurine can be reacted with a salt of azide (e.g., sodium azide, potassium azide, lithium azide) at elevated temperature in DMF to afford the 6-azido-3-deazapurine, which can be exposed to hydrogenation conditions to convert the 6-azide to a 6-amino group to produce 3-deazaadenine. This latter reaction may be performed in two steps or alternatively, in a single pot reaction to produce 3-deazaadenine in reasonably high yield (75+%, preferably 80+%).

Compounds according to the present invention may be used in pharmaceutical compositions having biological/pharmacological activity for the treatment of, for example, viral infections, in particular, Orthopox viruses as well as the SARS virus, as well as a number of other conditions and/or disease states which may appear or occur secondary to the viral infection. These compositions comprise an effective amount of any one or more of the compounds disclosed hereinabove, optionally in combination with a pharmaceutically acceptable additive, carrier or excipient. Compounds according to the present invention may also be used as intermediates in the synthesis of compounds exhibiting biological activity as well as standards for determining the biological activity of the present compounds as well as other biologically active compounds.

The compositions of the present invention may be formulated in a conventional manner using one or more pharmaceutically acceptable carriers. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally, or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutical compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions may be formulated in an ointment such as petrolatum.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of novel nucleoside of the instant invention that may be combined with the carrier materials to produce a single dosage form will vary depending upon the host treated, the particular mode of administration. Preferably, the compositions should be formulated so that a dosage of between about 0.01 and 150, preferably about 0.5 to about 25 mg/kg of patient/day of the novel nucleoside can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal and suppository administration, among other routes of administration. Enteric coated oral tablets may also be used to enhance bioavailability of the compounds from an oral route of administration. The most effective dosage form will depend upon the pharmacokinetics of the particular agent chosen as well as the severity of disease in the patient. Oral dosage forms are particularly preferred, because of ease of administration and prospective favorable patient compliance.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral or parenteral. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavouring agents, preservatives, colouring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques. The use of these dosage forms may significantly the bioavailability of the compounds in the patient.

For parenteral formulations, the carrier will usually comprise sterile water or aqueous sodium chloride solution, though other ingredients, including those which aid dispersion, also may be included. Of course, where sterile water is to be used and maintained as sterile, the compositions and carriers must also be sterilized. Injectable suspensions may also be prepared, in which case appropriate liquid carriers, suspending agents and the like may be employed.

Liposomal suspensions (including liposomes targeted to viral antigens) may also be prepared by conventional methods to produce pharmaceutically acceptable carriers. This may be appropriate for the delivery of free nucleosides, acyl/alkyl nucleosides or phosphate ester pro-drug forms of the nucleoside compounds according to the present invention.

In particularly preferred embodiments according to the present invention, the compounds and compositions are used to treat, prevent or delay the onset of viral infections of mammals and in particular Orthopox viruses, including alastrim vaccnia, variola, cowpox (smallpox), ectromelia, monkeypox and rabbitpox, as well as SARS coronavirus in mammals, especially including humans. Preferably, to treat, prevent or delay the onset of a viral infection, the compositions will be administered in oral dosage form in amounts ranging from about 250 micrograms up to about 500 mg or more at least once a day, preferably, up to four times a day, within the dosage range used for therapeutic treatment. The present compounds are preferably administered orally, but may be administered parenterally, topically, in suppository or other form.

In addition, compounds according to the present invention may be administered alone or in combination with other agents, including other compounds of the present invention. Certain compounds according to the present invention may be effective for enhancing the biological activity of certain agents according to the present invention by reducing the metabolism, catabolism or inactivation of other compounds and as such, are co-administered for this intended effect.

As indicated, compounds according to the present invention may be administered alone or in combination with other anti-viral agents for the treatment of a virus infection as otherwise described herein, especially including other compounds of the present invention or compounds which are otherwise disclosed as being useful for the treatment of Orthopox viruses, the SARS coronavirus, or other viruses. Certain compounds according to the present invention may be effective for enhancing the biological activity of certain agents according to the present invention by reducing the metabolism or inactivation of other compounds and as such, are co-administered for this intended effect.

The present invention is now described, purely by way of illustration, in the following further synthetic chemical description and the attached examples. It will be understood by one of ordinary skill in the art that these examples are in no way limiting and that variations of detail can be made without departing from the spirit and scope of the present invention.

### Synthesis of Novel Cyclopentenoside Compounds (5-Membered Ring) of the Present Invention

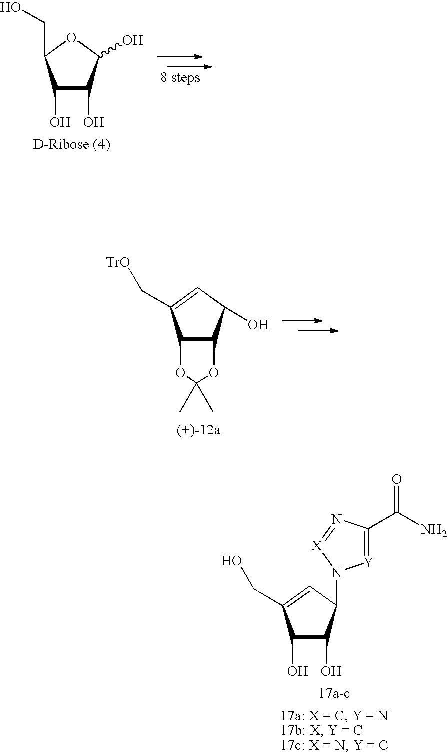

We describe herein an efficient and practical method for the synthesis of cyclopentenol (+)-12a from D-ribose (4) and its utilization for the synthesis of novel biologically active five-ring heterocyclic analogs (17-a-c) as potential antiviral agents.

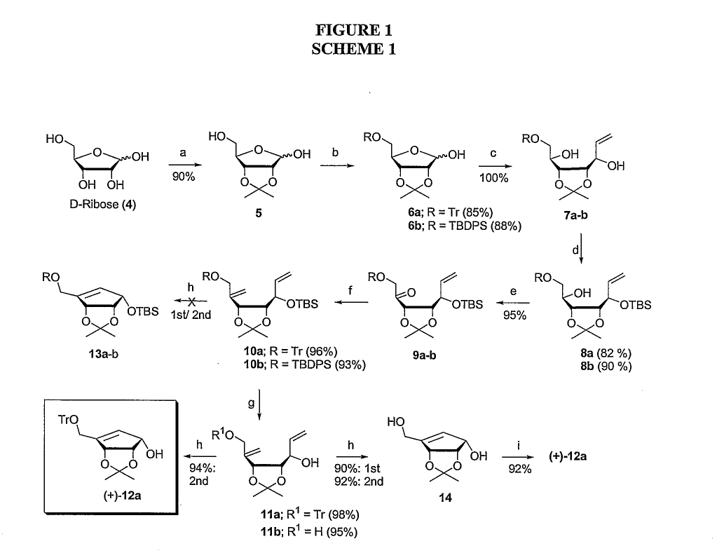

The chiral intermediate 7a was synthesized according to the published procedures⁶ as shown in scheme 1 (FIG. 1). D-ribose (4) was treated with 2,2-dimethoxypropane in the presence of a catalytic amount of p-toluenesulfonic acid to give isopropylidene derivative 5 in 90% yield, followed by the protection of the primary alcohol with triphenylmethyl (Tr) chloride to provide 6 in 85% yield. The protected lactol 6a was reacted with vinylmagnesium bromide to quantitatively give a single diastereomeric diol 7a, which was subsequently protected with tert-butyldimethylsilyl (TBDMS) only at the allylic hydroxyl position to afford silyldienol 8a in 82% yield, which was observed as two conformers by NMR spectrum as previously observed.¹²

To incorporate another double bond for the RCM reaction, the protected secondary alcohol 8a was oxidized to the ketone 9a by Swern oxidation, followed by Wittig reaction with methyltriphenylphosphonium bromide and n-BuLi in THF to provide the diene 10a in quantitative yield. With the diene 10a in hand, RCM reaction was investigated in the presence of 10 or 20 mol % 1st/2nd Grubbs catalysts without success, providing trace amounts of the desired tri-substituted diene 13a (Scheme 1). The literature search indicated that few RCM reaction with hindered terminal double bonds, as in the case of diene 10a-b, has been successful with the Grubbs catalysts, due to the significant steric hindrance by bulky groups, the major obstacle of the RCM reaction.¹³ Therefore, the silyl group from diene 10a was removed with TBAF to give the less sterically demanding dienol 11a, which was successfully converted to an cyclopentenol (+)-12a with only 2.0 mol % of the 2^{nd}-generation catalyst in 94% yield (whereas 40.0 mol % of 1^{st}-generation catalyst in 90%), thus provided the RCM reaction for the synthesis of multigram scale (10 g) cyclopentenol (+)-12a without any major difficulties.

Alternative approaches for the substrate with unprotected dihydroxyl moiety (11b) were also investigated to minimize the amount of the Grubbs catalysts in the RCM reaction as shown in Scheme 1 [Method B]. Thus, dienol lib was prepared from 5 following the same route: 2′,3′-protected D-ribose 5 was reacted with tert-butyldiphenylsilyl chloride (TBDPSCl) in the presence of imidazole in CH₂Cl₂ to obtain a silyl lactol 6b in 90% yield. Grignard reaction of the lactol 6b, followed by subsequent selective protection with TBDMSCl, Swern oxidation and Wittig reaction afforded bis-silyl diene 10b in 80% overall yield. Two silyl groups of 10b were then removed with TBAF to obtain dihydroxydiene 11b as the substrate for the RCM reaction. The diene 11b was then cyclized to provide the cyclopentenol 14 with 5.0 mol % of 2^{nd}-generation catalyst or 10.0 mol % of 1^{st}-generation catalyst in 92% and 90% yields, respectively. The primary alcohol 14 was reacted with TrCl in the presence of Et₃N in CH₂Cl₂ to give the mono-protected cyclopentenol (+)-12a in 92% yield. Thus, the best optimal condition for cyclopentanol (+)-12a via RCM reactions was with substrate 11a or 11b in the presence of 2.0 mol % of the 2^{nd}-generation Grubbs catalyst.

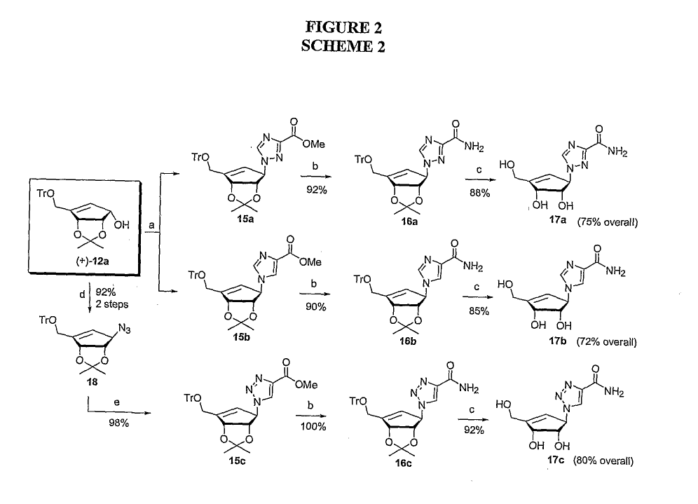

The stereochemistry of cyclopentenol (+)-12a was determined based on ¹H-NMR spectra, Nuclear Overhauser Effect (1D-NOE) and [α]^{D} value, which were compared to the reported values¹⁴ for (+)-12a along with the data of its diastereomer (+)-12b, which was readily converted from (+)-12a by Mitsunobu reaction¹⁵. Additionally, 1D-NOE was determined in which a significant interaction between H^{a} and H^{c} in (+)-12a was observed, whereas the NOE between H^{e} and H^{c} was not observed in (+)-12b as shown in FIG. 2.

With the chiral (+)-12a in hands, Mitsunobu reaction¹⁶ of (+)-12a with methyl-1H-1,2,4-triazole-3-carboxylate in the presence of DIAD and PPh₃ was reacted to obtain the triazole nucleoside 15a (Scheme 2). The ester 15a was transformed to the amide 16a with saturated methanolic ammonia, followed by deprotection of trityl and isopropylidene groups by methanolic hydrogen chloride solution (1.0 M HCl in diethylether) to afford the desired nucleoside 17a in 75% yield in three steps from (+)-12a. The other five-ring heterocyclic nucleosides 17b was also prepared from the coupling reactions of (+)-12a with methyl-4-imidazolecarboxylate by the similar method in 72% yield. The 1,2,3-triazole derivative (17c) was also synthesized using the 1,3-dipolar reaction of methyl propiolate with the azide derivative (18), prepared from (+)-12a by the reported method.¹⁷ The ester 15c was converted to the amide 16c in saturated methanolic ammonia. which was treated with methanolic hydrogen chloride to afford 1,2,3-triazole carbocyclic nucleoside 17c in 80% overall yield from (+)-12a.

The newly synthesized carbocyclic nucleosides (17a-c) have been evaluated for their antiviral activity against vacinnia, cowpox and SARS viruses, and the results are summarized. The 1,2,3-triazole analogue (17c) exhibited the most potent antiviral activity (EC₅₀ 0.4 μM) against vacinnia virus with high selectivity (SI>750) and moderate activity (EC₅₀ 39 μM, SI>7.7) against cowpox virus as well as weak activity (EC₅₀ 47 μM, SI>2.1) against SARS virus. The 1,2,4-triazole analogue (17a) also exhibited comparable antiviral activity (EC₅₀ 21 μM, SI>4.8) against SARS virus, whereas the imidazole analogue (17b) did not show any significant antiviral activity.

In summary, an efficient synthetic methodology for the cyclopentenol (+)-12a, employing the RCM reaction with the minimum amount of 2^{nd}-generation Grubbs catalyst, has been developed for a multi-gram scale. Coupling reactions of cyclopentenol (+)-12a with appropriate five-membered ring heterocycles provided novel antiviral agents of biodefense interest.

### Efficient Synthetic Method for 3-Deazaadenine

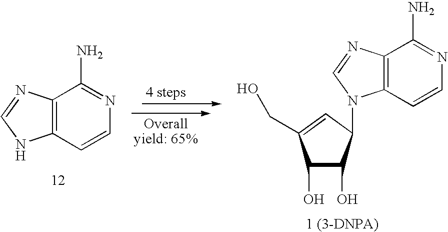

The key intermediate, 3-deazaadenine 12 was prepared from 4-chloroimidazo[4,5-c]pyridine 6 with LiN₃ and DMF-[emim]BF₄, from which a practical synthesis of (−)-3-deazaneplanocin A (1) was accomplished via Mitsunobu reaction.

We developed a significantly improved synthesis of 3-deazaadenine (13) and the condensation reaction (Mitsunobu reaction) for the synthesis of 3-deazaneplanocin A (1) in a multi-gram scale. See Scheme 1A.

The key intermediate 6 was synthesized according to the modified method by the previously reported procedures from commercially available 4-amino-2-chloropyridine (5)⁸; N-nitration and its rearrangement followed by hydrogenation to give 2-chloro-3,4-diaminopyridine, which was then reacted with acetic anhydride and triethylorthoformate to furnish 6-chloro-3-deazapurine (6) as shown in Scheme 1A.

With the key heterocyclic intermediate 6 in hand, the Mitsunobu reaction of 6 with the chiral cyclopentenyl moiety (4),⁹ prepared from D-ribose (3), was carried out to obtain a mixture of N₉- and N₇-regioisomers (7 to 8, 2:1) in 95% yield, which was difficult to separate [isolated yields; 7 (20%) and 8 (18%)]. The ratio of the two isomers was determined by ¹H-NMR, and their configuration was identified by Nuclear Overhouser Effect (1D-NOE), which indicated the interaction between the Cl′—H and the aromatic C3-H of compound 7, whereas the same effect was not present in compound 8. The desired protected nucleosides 7, purified from the reaction mixture, was converted to 3-DNPA 1 by deprotecting the trityl and isopropylidene groups with methanolic HCl, followed by a substitution reaction with hydrazine and hydrogenation in the presence of Raney-nickel. The N₇-isomer 8 was also converted to the corresponding 3-DNPA analogue 10 in 48% yield by the same procedure. The λₘₐₓ values of synthesized 3-DNPA (1) were showed at 265 and 217 nm, whereas those of N₇-3-DNPA 10 were appeared at 285 and 212 nm, which is consistent with the previously reported values.^{3a} The regioselectivity of 6-chloro-3-deazaadenine (6) of the Mitsunobu reaction was inferior to 6-chloroadenine and the yield of amination reaction of 6-chloro-3-deaazaneplanocin (9) to 3-deaza-nepanocin A were lower than that of neplanocin A.¹⁰

To improve the selectivity of N₉-substitution by blocking the N₇-position with sterically demanding groups, 6-chloro-3-deazapurine (6) was converted to 3-deazaadenine derivatives (14a-d) (Scheme 2). Our initial attempts to convert the 6-chloride to the N⁶-amino group by methanolic ammonia or hydrazine/Raney-Nickel gave 3-deazaadenine (12) in poor yields. However, the reaction of 6 with NaN₃ and LiN₃ provided 3-deza-tetrazolopurine (11) in 80 and 82% yields, respectively (Table 1). The same reaction for 3-deaza-tetrazolopurine (11) was investigated with addition of an ionic liquid (DMF-[emim]BF₄).¹¹ LiN₃ (20% in water) and DMF-[emim]BF₄ ionic liquid (10:1 v/v) at 80° C. for 6 h provided 3-deaza-tetrazolopurine (11) in quantitative yield. The ionic liquid was readily recovered by simple filtration, and recycled.¹²

TABLE 1
The result of synthesis of 3-deazaadenine 12

| Entry | Starting material | Reaction conditions | Yield (%) |
|---|---|---|---|
| 1 | 6 | NH₃, MeOH, 150° C., 48 h | ? |
| 2 | 6 | NH₂NH₂/Raney-nickel | 45 |
| 3 | 6 | Na N₃, DMF, 140° C., 48 h; | 80 |
| | | H₂, Pd/C | |
| 4 | 6 | 20 wt % of N₃Li in H₂O | 82 |
| | | DMF, 130° C., 48 h; H₂, Pd/C | |
| 5 | 6 | 20 wt % of LiN₃ in H₂O | >98 |
| | | DMF-[emim]BF₄ (10:1 v/v), | |
| | | 80° C., 6 h; H₂, Pd/C | |

The 3-Deaza-tetrazolopurine (11) was converted to the 3-deaza-6-aminopurine derivative 12 by h 94%

yield, respectively. Two N-benzoyl groups from tris-N-benzoyl-3-deazaadenine (13a) were removed by the previously reported method¹⁵ to give mono-N⁶-benzoyl-3-deazaadenine 14a in 85% yield, and the removal of one N-benzoyl group with 1.0 M NaHCO₃ in THF gave bis-N⁶-benzoyl-3-deazaadenine 14b in 54% yield.^{14a} Also, tris-N-Boc-3-deazaadenine 13b was converted to mono-N⁶-Boc-3-deazaadenine 14c and bis-N⁶-Boc-3-deazaadenine 14d by NaOMe and NaHCO₃ in 96% and 92% yields, respectively.^{16,14a}

With protected 3-deazaadenine derivatives 14a-d in hand, the coupling reaction with the cyclopentenyl moiety 4 in the presence of DIAD, Ph₃P and THF was investigated and is summarized in Table 2. Unexpectedly, the reaction of mono-N⁶-benzoyl-3-deazaadenine (14b) with 14a gave only 94% of the N₇-isomer 16a without detectable amounts of N₉-isomer, whereas bis-N⁶-benzoyl-3-deazaadenine 14b gave exclusively the N₉-isomer 15b in 92% yield under similar reaction conditions. The Mitsunobu reaction of mono-N⁶-Boc-3-deazaadenine (14c) and 4 provided a mixture of the N₉- and N₇-isomers (4:1) in 83% yield. In the case of bis-N⁶-Boc-3-deazaadenine (14d), however, the selectivity toward the N₉-isomer (15d) over the N₇-isomer (15d) increased to 10:1 in 88% yield. The major product N₉-isomer 15d was readily separable from the reaction mixture by silica-gel column chromatography.¹⁷

TABLE 2
The results of Mitsunobu reaction and overall yield of 3-DNPA
(1) and N7-3-DNPA (10) from 6

| | | Ratio of N9:N7 | |
|---|---|---|---|
| | | (combined yield, | Overall % yield |
| Entry | Compound | %) | from 6 |
| 1 | 6 | 2:1 (94)^{a} | 10 (1) |
| 2 | X = H, Y = Bz (15a) | 0:10 (94)^{b} | 70 (10) |
| 3 | X, Y = Bz (15b) | 10:0 (92)^{b} | 41 (1) |
| 4 | X = H, Y = Boc (15c) | 4:1 (83)^{a} | 34 (1) |
| 5 | X, Y = Boc (15d) | 10:1 (88)^{b} | 65 (1) |

^{a}The ratio determined by ¹H-NMR.
^{b}The ratio isolated by silica gel column.

Thus, in view of the regiospecifc ratio as well as the overall yield of the desired product, bis-N⁶-Boc-3-deazaadenine (14d) was proved to be the best substrate for the synthesis 3-DNPA 1, which was used for the preparative scale synthesis. The removal of Boc, trityl and isopropylidene groups from 15d by methanolic hydrogen chloride gave 3-deazaneplanocin A (1) in 65% overall yield. Additionally, N₇-3-DNPA (10), a regioisomer of 3-DNPA 1 was also obtained in 70% overall yield from 6.

In summary, an efficient synthetic methodology for a gram-scale synthesis of 3-deazaneplanocin A (1) has been developed. We also developed a multi-gram synthesis of 3-deazaadenine 12 from 6-chloro-3-deazaadenine by using an ionliqid. These observed interesting regiospecific Mitsunobu reactions of the 3-deaza-adenine derivatives warrant, further investigation of the underlying mechanistic studies.

### Synthesis and Antiviral Activity of 7-Deaza Neplanocin A Against Orthopox Viruses (Vaccinia and Cowpox Virus)

The preparation of 7-deaza neplanocin A (2) is shown in Scheme 1C. The cyclopentenol 3, which was prepared according to the literature with slight modifications of reported method, 12 was coupled with 6-chloro-7-deazapurine 13 under the Mitsunobu reaction conditions to give compound 4 in 81% yield. Treatment of 4 with methanolic ammonia at 100° C. for 12 h provided compound 5 in 83% yield. Deprotection in acidic conditions of 5 in 10% HCl in methanol followed by neutralization with NaHCO3 gave 7-deaza neplanocin A (2) 14 in 63% yield.

The antiviral activity of 7-deaza neplanocin A (2, Scheme 1C) was evaluated against a wide variety of viruses; including 80 cowpox, vaccinia, yellow fever, dengue type 2, Punta Toro A, SARSCoV, Tacaribe, VEE, and West Nile. Among the tested viruses, 7-deaza neplanocin A exhibited potent activity against cowpox and vaccinia viruses in a CPE reduction assay without any significant cytotoxicity in HFF cells as shown in the Table below. Although Neplanocin A has potent broad spectrum antiviral activity including orthopox viruses,² significant cytotoxicity of NPA limited its usefulness as an antiviral agent.¹⁵ However, 7-deaza NPA (2) did not show any 90 cytotoxicity up to 300 μM in HFF cells in a neutral red assay. Furthermore, 7-deaza NPA was more potent than that of cidofovir in this assay, which has been known to be one of the most potent agents against orthopox viruses.¹⁶

In summary, the synthesis of 7-deaza neplanocin A (2) was afforded by the coupling of functionalized cyclopentenol (3) with 7-deazapurine. The synthesized 7-deaza neplanocin A (2) showed potent antiviral activity against orthopox viruses (cowpox and vaccinia) without any significant cytotoxicity. Further biological evaluation to delineate 100 the mode of action as well as to study in animal models to assess the full potential of 7-deaza neplanocin A is warranted.

TABLE
Antiviral activity of 7-deaza neplanocin A (2) against cowpox
and vaccinia viruses

| | | EC50 | EC90 | Cytotoxicity | Cidofovira^{a} |
|---|---|---|---|---|---|
| Virus | Cell line | (μM) | (μM) | CC50 (μM) | EC50 (μM) |
| Cowpox | HFF cells | 1.2 | 4.6 | >300 | 5.0 |
| Vaccinia | HFF cells | 3.4 | 21.5 | >300 | 4 |

^{a}Positive control.

### Synthesis of 7-Deaza-7-Substituted Neplanocin A

The synthesis of halogen (F, Cl, Br, I) substituted derivatives is presented in Scheme 1 below. The Mitsunobu coupling of cyclopentenol 1 and 7-halogenated-7-deaza-6-chloropurine (2, 3, 4 and 5) which were prepared followed by known method gave the coupling products. Those coupling products were purified by column chromatography and subjected to the ammonolysis reaction to give compound 6, 7, 8 and 9 respectively. The acidic hydrolysis of 6, 7, 8 and 9 by HCl in warm MeOH and THF gave 10, 11, 12 and 13 respectively.

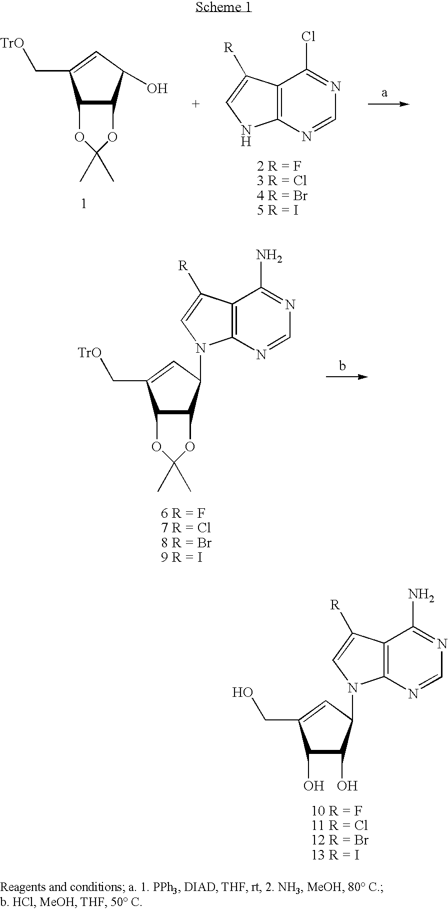

The synthesis of CN and CONH₂ substituted derivatives (17 and 18) were presented in Scheme 2, below. The Mitsunobu coupling of cyclopentenol 1 and 7-iodo-7-deaza-6-chloropurine 5 gave the coupling product 14. The iodo group in 14 was converted to CN by palladium catalyzed reaction to give 15 in moderate yield. The ammonolysis of 15 followed by acidic hydrolysis by HCl in warm MeOH and THF gave 17. The CN group of 17 was converted to CONH₂ by the treatment with H₂O₂ in ammonium hydroxide to give 18 in quantitative yield.

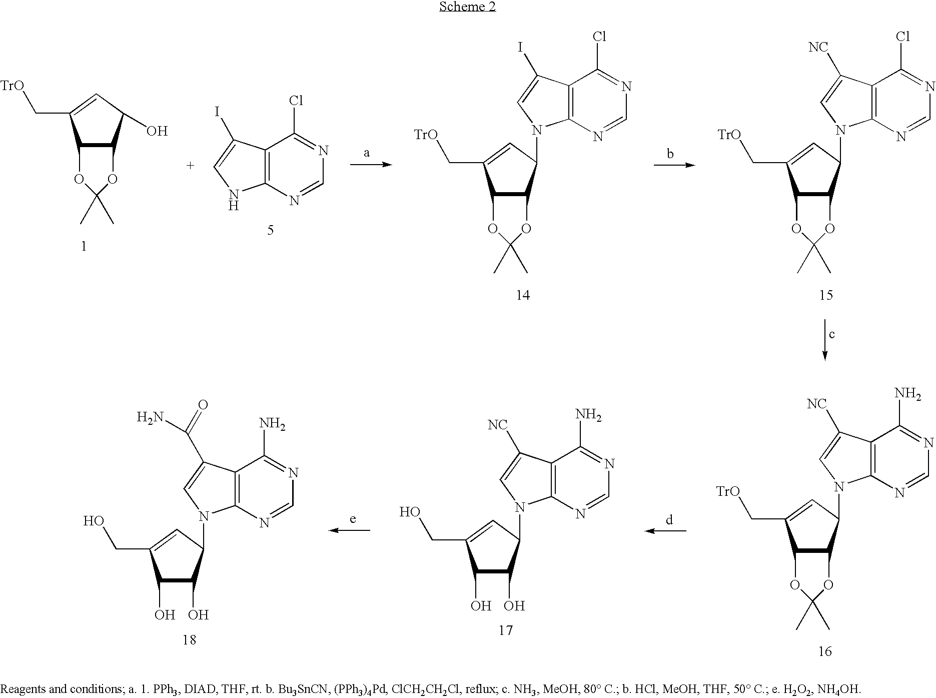

The synthesis of NO₂ and NH₂ substituted derivatives (22 and 23) is presented in Scheme 3, below. The acetylation of 7-deaza NPA gave a mixture of 20a and 20b. The mixture was treated with a mixture of fuming nitric acid and sulfuric acid in dichloromethane to give 21 in good yield. The acidic hydrolysis of 21 by HCl in warm MeOH and THF gave 22. The nitro group of 22 was reduced to amine by Raney Ni and hydrazine in aqueous MeOH to give 23, but the compound 23 was not stable which was decomposed to very complex mixture over several days.

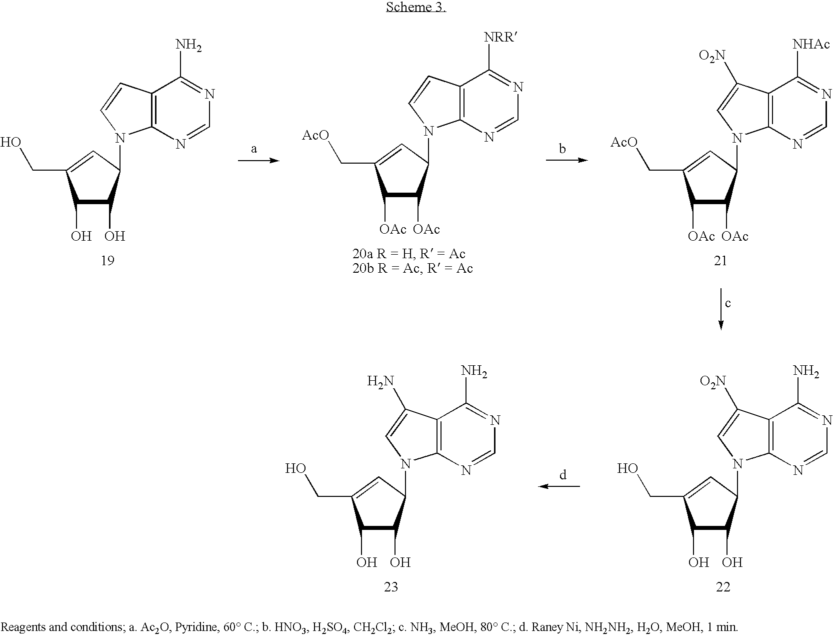

The synthesis of vinyl and acetylene substituted derivatives (25 and 27) were presented in Scheme 4 and Scheme 5. Initially the iodo compound 14 was converted to the vinyl (24) and acetylene derivatives (25) by palladium catalyzed reaction followed by ammonolysis. However, the acidic hydrolysis of 24 and 26 by HCl in warm MeOH and THF gave only very complex mixture which could not be separated. To avoid the final acidic deprotection step, acetyl protected cyclopentenol 29 was prepared from 28 by acetylation and DDQ deprotection of PMB group. Then, the Mitsunobu coupling of cyclopentenol 29 and 7-iodo-7-deaza-6-chloropurine 5 gave the coupling product 30. The iodo group in 30 was converted to the vinyl and TMS protected acetylene by palladium catalyzed reaction to give 31 and 32 respectively. The ammonolysis of 31 and 32 provided the desired compound 25 and 27 in good yield.

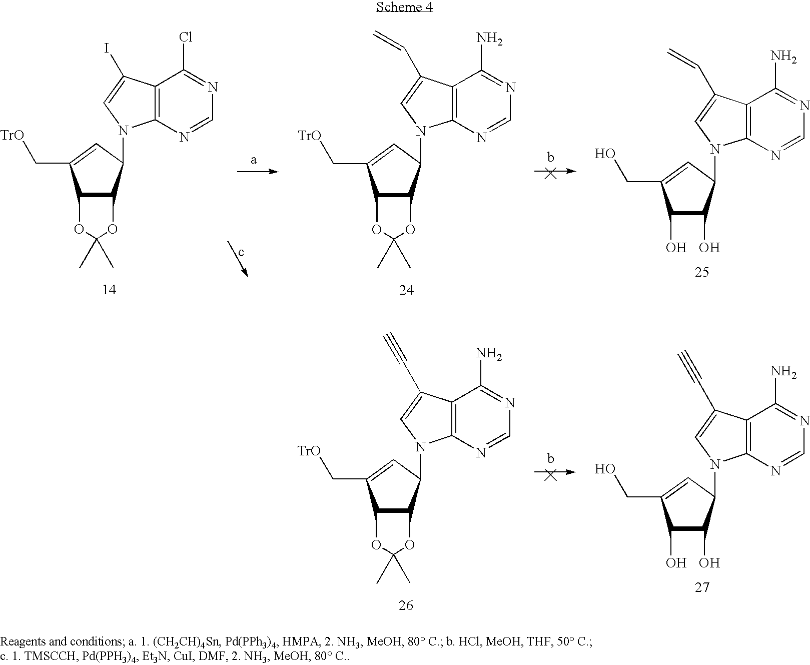

The synthesis of 7-deaza guanine derivative 37 was presented in Scheme 6. The Mitsunobu coupling of known cyclopentenol 33 and N-isobutyro-7-deaza-2-amino-6-chloropurine 34 followed by TBAF treatment gave the compound 35. Compound 35 was refluxed in 2N NaOH overnight gave 7-deaza guanine compound 36 in good yield. The acidic hydrolysis of 36 by HCl in MeOH gave 37.

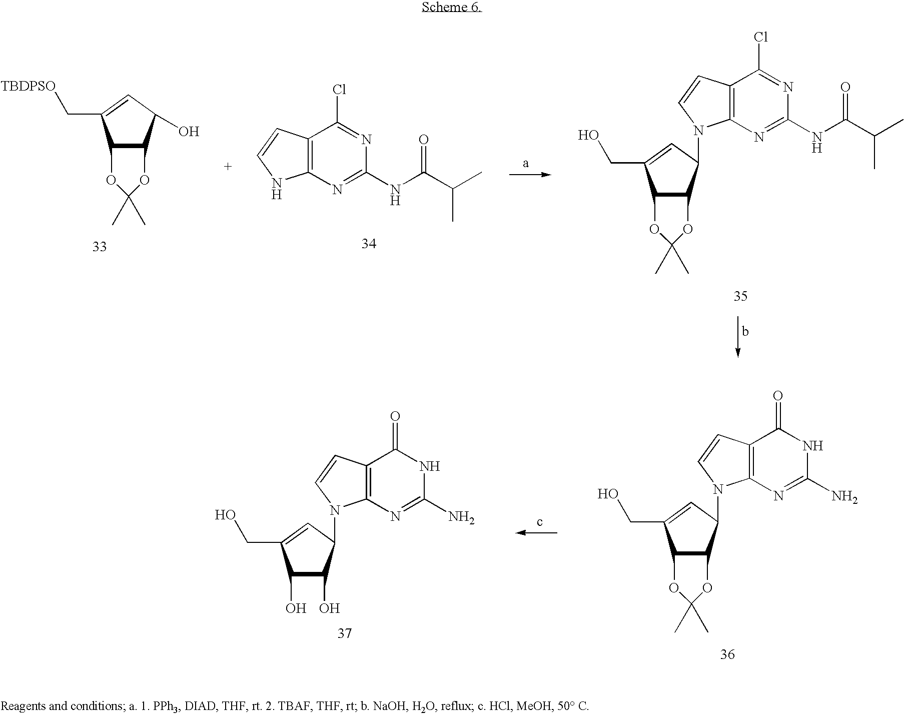

### 2′-Deoxy carbocyclic cyclopentenyl nucleosides

For a convergent synthesis of 2′-deoxy carbocyclic cyclopentenyl nucleoside, the key intermediate 42 was condensed with pyrimidine and purine bases.

The preparation of 2′-deoxy intermediate 42 is shown in Scheme 7 below. Cyclopentenol derivative 1 was synthesized according to the procedure recently developed from our laboratory and converted to it's benzyl ether 38. The benzyl ether compound 38 was hydrolyzed to triol 39. Two hydroxy groups of the triol 39 were regioselectively protected by TIPDSCl to afford compound 40. Hydroxy group of 40 was converted to bromide by triflate formation followed by nucleophilic substitution reaction. For the nucleophilic substitution of the triflate, the selection of the reaction media was critical. When HMPT, highly aprotic polar solvent, was used as solvent, the reaction was complete in 1 h at rt. However, the resulting bromide was an inseparable mixture of two compounds (α and β) and one of them was inert to the next reaction condition (Bu₃SnH/AIBN). When THF or dioxane, weakly polar solvent, was used as solvent, the reaction was too slow. The optimum solvent was found to be acetonitrile which gave only one product with acceptable reaction time and yield. (36 h at rt, 93%) The key intermediate 42 was prepared by bromide reduction by standard radical reaction condition (Bu₃SnH/AIBN) followed by debenzylation with Na/NH₃.

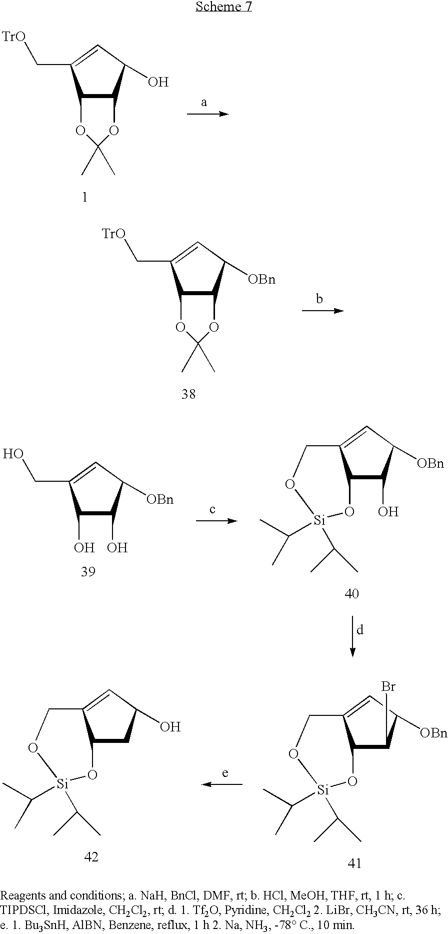

Coupling of 42 with purine and pyrimidine bases was carried out with Mitsunobu reaction. (Scheme 8) For the synthesis of pyrimidine analogues, the cyclopentenyl alcohol 9 was treated with N³-benzoyluracil and N³-benzoylthymine in the presence of DIAD and PPh₃ in THF. The resulting coupling products were subjected to debenzoylation condition (saturated NH₃ in MeOH) to give 43 and 47, respectively. Desilylation of the TIPDS group with Et₃N.3HF gave the uracil nucleoside 44 and thymine nucleoside 48, respectively. The uracil base in 43 was converted to the corresponding cytosine by the reported method¹² followed by acidic deprotection of TIPDS group by HCl to yield the cytosine nucleoside 46.

The Mitsunobu reaction of 42 with 6-chloropurine and 2-amino-6-chloropurine followed by desilylation with Et₃N.3HF gave compound 49 and 52, respectively. Treatment of 49 with NH₃ in MeOH at 70° C. gave the adenine nucleoside 50. Treatment of 49 with mercaptoethanol and sodium methoxide in refluxing MeOH afforded the hypoxanthin nucleoside 51. The guanine nucleoside 53 was prepared from 52 with 2-mercaptoethanol and sodium methoxide in refluxing MeOH.

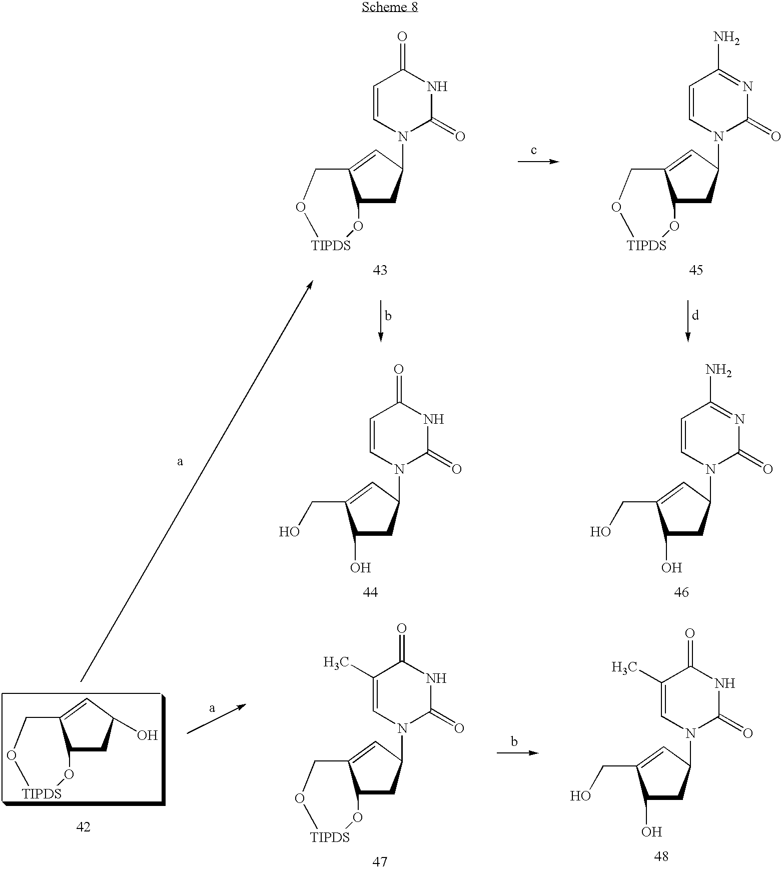

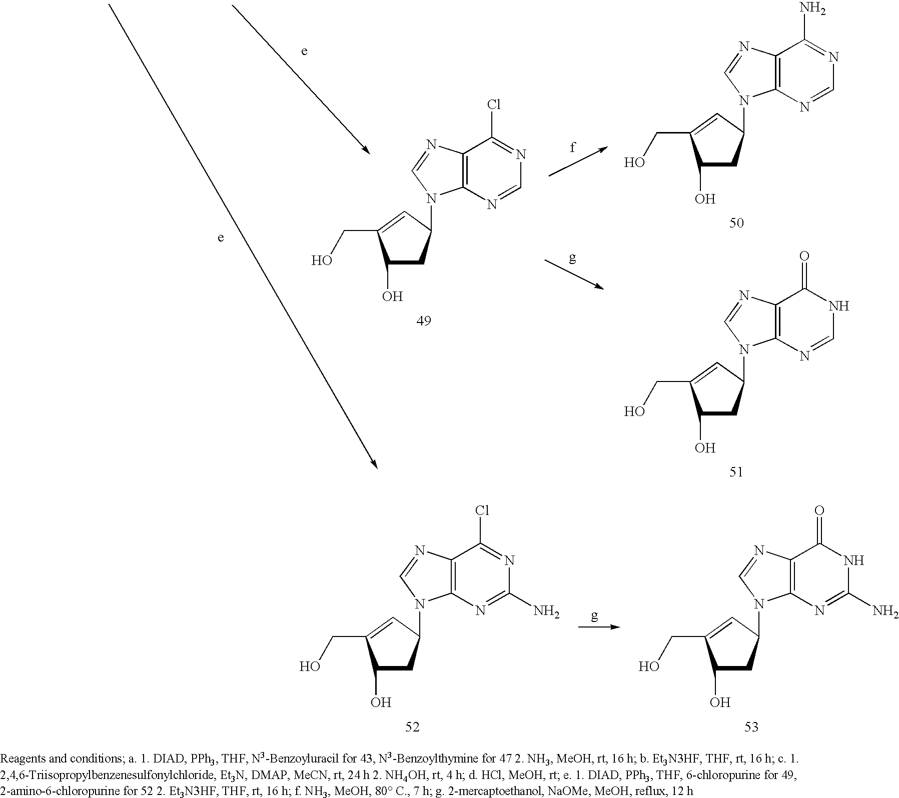

### 2′-Deoxy-2′-fluoro carbocyclic cyclopentenyl nucleosides

For a convergent synthesis of 2′-deoxy-2′-fluoro carbocyclic cyclopentenyl nucleoside, we condensed the key intermediate 57 with pyrimidine and purine bases.

The preparation of 2′-deoxy-2′-fluoro intermediate 57 is shown in Scheme 9. Cyclopentenol 1 was converted to it's PMB ether and hydrolyzed by HCl in methanol and THF to give the triol 28. The primary hydroxyl group was selectively protected with trityl ether to give 54. The allylic OH from the diol 54 was selectively protected by the orthoformate formation and DIBAL reduction to yield 55 in modest yield. The compound 55 was converted its O-triflate by triflic anhydride and pyridine and the triflate was treated with TBAF in THF to give the desired fluoro compound 56 in good yield. The PMB group in 56 was removed by DDQ in wet CH₂Cl₂ to give the key intermediate 57.

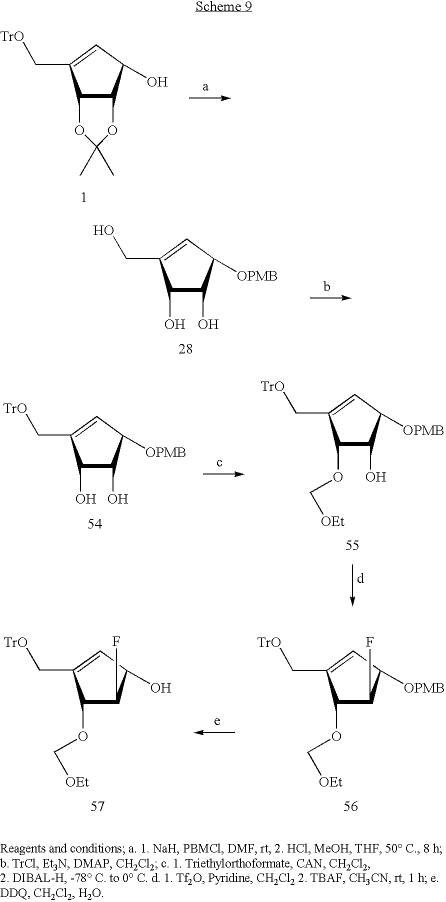

Coupling of 57 with purine and pyrimidine bases was carried out with Mitsunobu reaction. (Scheme 10, below). For the synthesis of pyrimidine analogues, the key intermediate 57 was treated with N³-benzoyluracil and N³-benzoylthymine in the presence of DIAD and PPh₃ in THF. The resulting coupling products were subjected to debenzoylation condition (saturated NH₃ in MeOH) to give 58 and 61, respectively. The uracil group in 58 was converted to cytosine by known method to give 59. The acidic deprotection of 59 and 61 by HCl in MeOH and THF gave the cytosine and thymine nucleosides 60, 60 respectively.

The Mitsunobu reaction of 57 with 6-chloropurine and ammonolysis gave compound 63. The Mitsunobu reaction of 57 with N-isobutyro-2-amino-6-chloropurine and subsequent treatment with NaOMe and 2-mercaptoethanol in refluxing methanol gave compound 65. The acidic deprotection of 63 and 65 by HCl in MeOH gave the adenine and guanine nucleosides 64, 66 respectively.

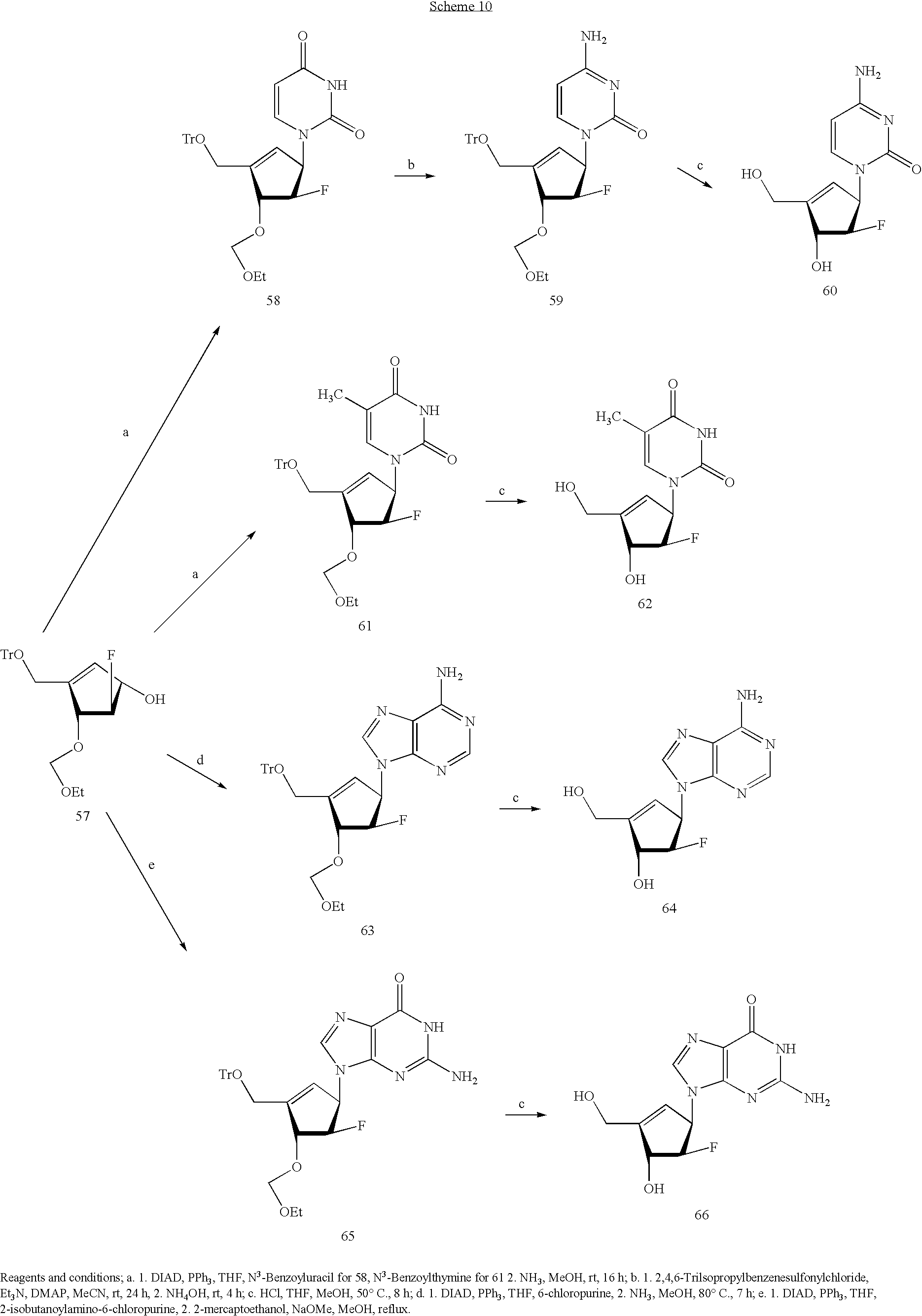

### Synthesis of 3′-deoxy carbocyclic cyclopentenyl nucleosides

The synthesis of 3′-deoxy cyclopentenyl sugar moiety 71 is presented in Scheme 11, below.

The triol 53 was treated with TBDPSCl to give the selectively protected compound 67. The syn-diol group of 67 was converted to epoxide group to give 68. Lithium aluminum hydride reduction of 68 gave compound 69 exclusively in excellent yield. The PMB group in 69 was removed by sequential reactions of benzoylation, DDQ reaction and NaOMe debenzoylation because the direct reaction of 69 with DDQ did not provide compound 70. The diol 70 was first treated with SOCl₂ then sodium azide in DMF for 3 hours and silylation to give the mixture of 71a and 71b as a 4:1 ratio which could not separated by chromatography. The formation of 71b was thought to be the result of 6π C electron rearrangement of azide addition product similar to Cope rearrangement. However, the exact nature (effect of pH, solvent or temperature on the rearrangement) of the rearrangement was not explored.

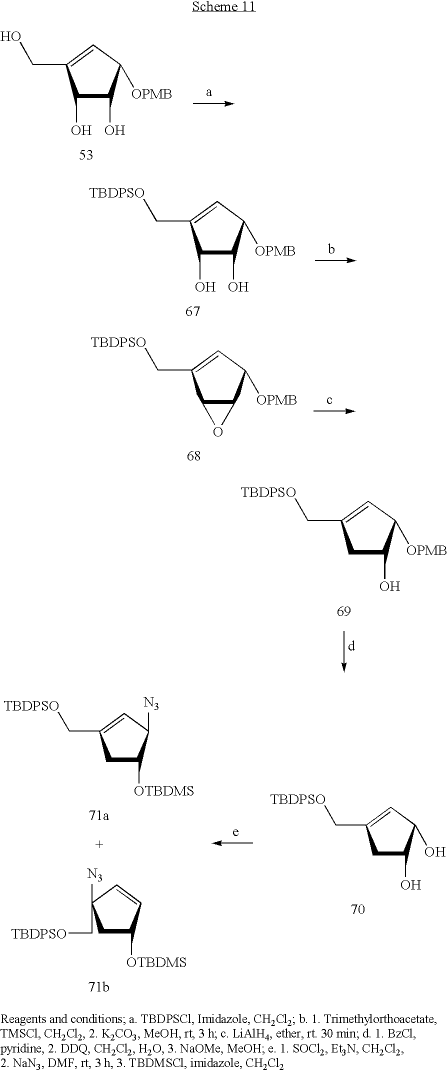

With the compound 71 in hand, 1,2,3-triazole type nucleoside were prepared. The azide group in compound 71 was converted to the triazole ring by copper catalyzed reaction to give 72 in good yield. Desilylation of 72 with TBAF in THF and subsequent ammonolysis of 73 gave 3′-deoxy-cyclopentenyl 1,2,3-triazole nucleoside 74.

It is well known that natural pyrimidine and purine bases can be built from an amine group. With this in mind, the reduction of azide in 71 by PPh₃ in refluxing methanol gave the corresponding amine. This amine compound was treated with acrylic isocyanate to give the precursor of uracil 75. However, the yield of this reaction was low (˜25%) so more efficient routes to the 3′-deoxy series are being explored.

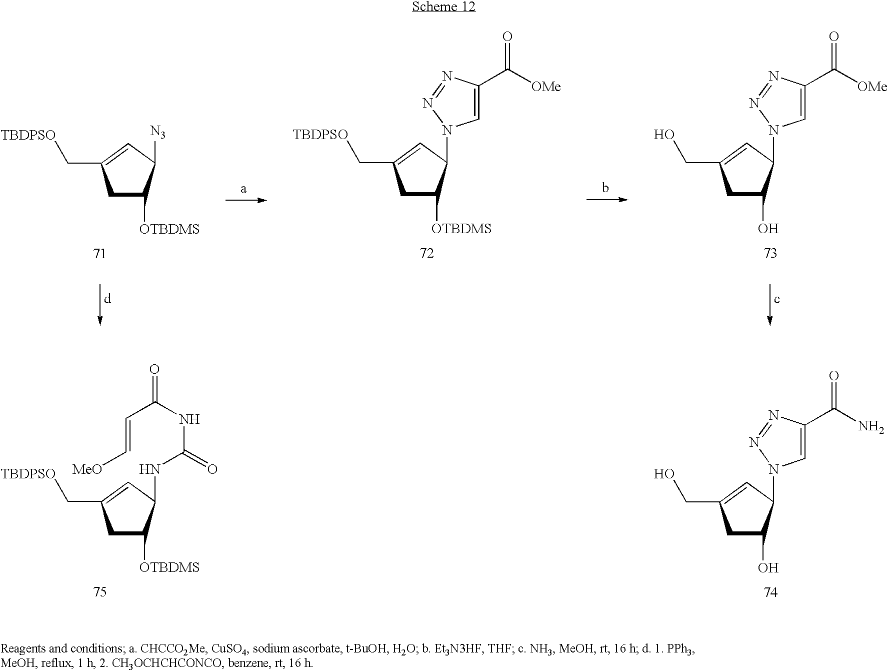

### Synthesis of 3-Deazaneplanocin A and 3-Deazaguanosine-3′-Deoxy Carbocyclic Cyclopentenyl Nucleosides

The chiral cyclopentenyl moiety 5 (below) was prepared from D-ribose in 8 steps following a previously reported synthetic method via a chiral induction, a regioselective protection of hydroxy group and ring-closing metathesis with 0.1 mole % of the 2^{nd} generation Grubbs catalyst as key steps.¹⁰

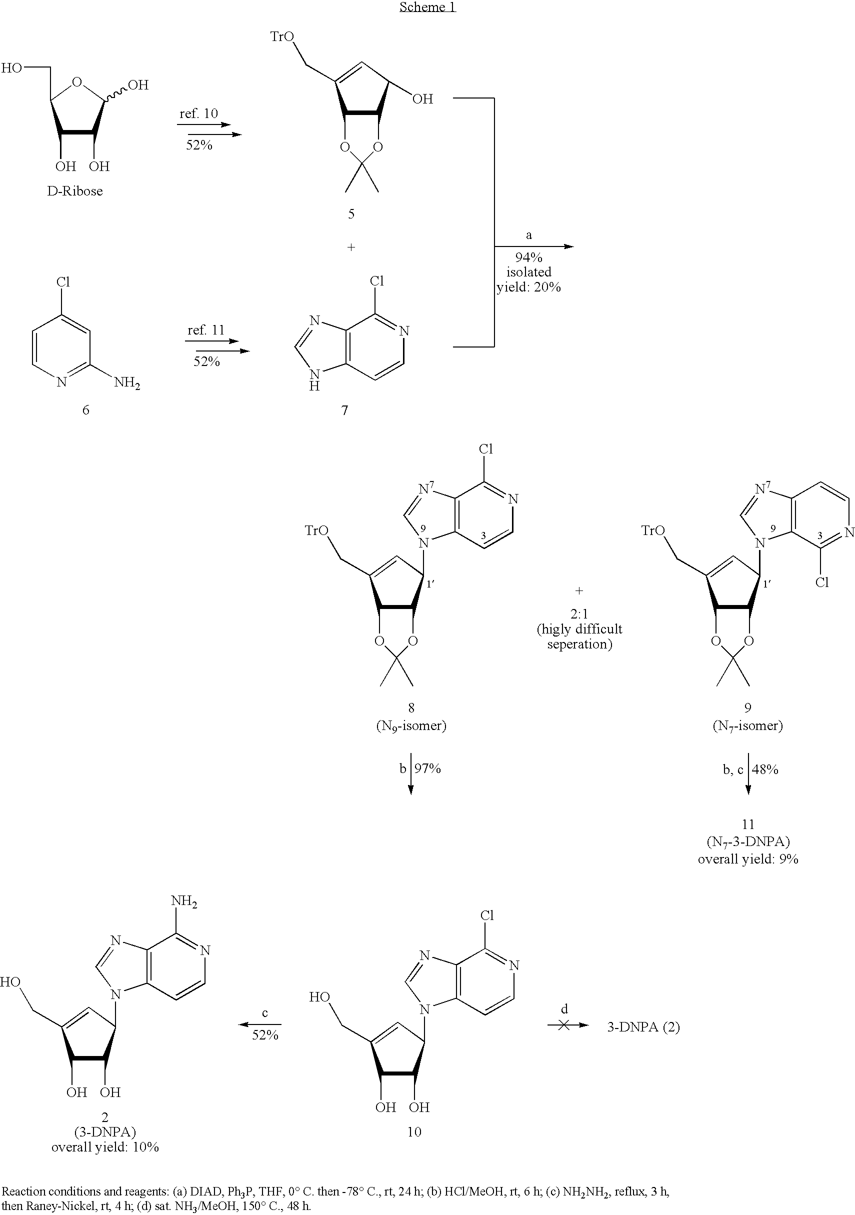

The other key intermediate 7 was synthesized according to the previously reported procedure¹¹ from commercially available 4-amino-2-chloropyridine (6) in 4 steps (overall 54% yield) as shown in Scheme 1. Mitsunobu reaction of 7 with 5 provided a mixture of the N₉- and N₇-regioisomers (8:9=2:1) in 94% yield. The separation of the desired product (8) from the reaction mixture was difficult by silica gel column [isolated yields: 20% (8) and 20% (9), respectively]. The ratio of the two isomers was determined by ¹H-NMR, and their configuration was identified by Nuclear Overhouser Effect (1D-NOE), which indicated the interaction between the C₁′—H and the aromatic C₃—H of compound 8, whereas the same effect was not present in compound 9.

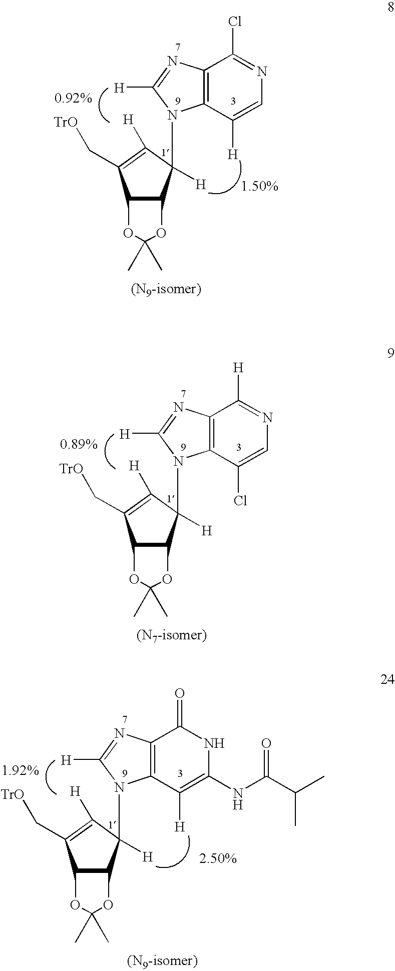

### Figure Studies of 1D-NOE

The purified product 8 from the reaction mixture was converted to 3-DNPA (2) by deprotecting the trityl and isopropylidene groups, followed by a substitution and hydrogenolysis in 50% yield (3 steps). However, compound 10 could not be converted to 2 under the condition of methanolic ammonia in a steel bomb. The N₇-isomer 9 was also converted to the corresponding 3-DNPA analogue 11 in 48% yield by the same procedure. The λₘₐₓ values of the synthesized 3-DNPA (2) were 264 and 212 nm, whereas those of N₇-3-DNPA (11) were 286 and 212 nm, which was consistent with the previously reported values.^{2a} The result showed that the regioselectivity (N9/N7) of 6-chloro-3-deazaadenine (7) for the Mitsunobu reaction was inferior to 6-chloroadenine.¹² Furthermore, the reactivity of 6-chloro-3-deazaneplanocin moiety (10) with ammonia in order to convert 10 to 3-DNPA (2) was less than that for the 6-chloro-neplanocin A analogue.¹²

To improve the selectivity for the N₉-substitution by blocking the N₇-position with sterically demanded groups, 6-chloro-3-deazapurine (7) was converted to N-protected 3-deazaadenine derivatives (21a-b) (Scheme 2). Our initial attempts to convert the 6-chloride to the N⁶-amino group by methanolic ammonia or hydrazine/Raney-nickel gave 3-deazaadenine (13) in poor yields. However, the reaction of 7 with NaN₃ or LiN₃ provided 3-deaza-tetrazolopurine (12)^{1a} in about 80% and 82% yields with about 20% of starting material (7), which could not be removed from 12 (Table 1), respectively. The same reaction for 3-deaza-tetrazolopurine (12) was investigated by addition of an ionic liquid (DMF-[emim]BF₄).¹³ LiN₃ (20% in water) and DMF-[emim]BF₄ ionic liquid (10:1 v/v) at 80° C. for 6 h provided 3-deaza-tetrazolopurine (12) in quantitative yield without 7. The ionic liquid was readily recovered by simple filtration, and recycled.¹⁴

TABLE 1
The result of synthesis of 3-deazaadenine (13)

| Entry | Starting material | Reaction condition | Yield of 13 (%) |
|---|---|---|---|
| 1 | 7 | NH₃, MeOH, 150° C., 48 h | —^{a} |
| 2 | 7 | NH₂NH₂/Raney-nickel | 45 |
| 3 | 7 | Na N₃, DMF, 140° C., 48 h; | 80^{b} |
| | | H₂, Pd/C | |
| 4 | 7 | 20 wt % of N₃Li in H₂O | 82^{b} |
| | | DMF, 130° C., 48 h; | |
| | | H₂, Pd/C | |
| 5 | 7 | 20 wt % of LiN₃ in H₂O | >95 |
| | | DMF-[emim]BF₄ (10:1 v/v), | |
| | | 80° C., 6 h; H₂, Pd/C | |

^{a}Starting material was recovered in 98%.
^{b}It was difficult to separate a desired product from remaining 7

The 3-deaza-tetrazolopurine (19) was converted to the 3-deazaadenine (13) by hydrogenolysis, followed by protection of the amino groups with di-tert-butyl dicarbonate [(Boc)₂O] afforded tris-N-Boc-3-deazaadenine, (14) in 94% yield. Tris-N-Boc-3-deazaadenine (14) was transformed into N⁶-Boc-3-deazaadenine (15a) with NaOMe in THF in 96% yield.^{15a} The removal of N₉-Boc group of 14 with 1.0 M NaHCO₃ in THF gave N⁶,N⁶-diBoc-3-deazaadenine (15b) in 92% yield.^{15b}
With protected 3-deazaadenine derivatives (15a and 15b) in hand, Mitsunobu reaction of 5 with 15a or 15b was carefully investigated in the presence of triphenyl phosphine (Ph₃P), diisopropyl azodicarboxylate (DIAD) and THF. The coupling reaction of N⁶-Boc-3-deazaadenine (15a) with 5 provided a mixture of N₉- and N₇-isomers (16a:17a=1:4) in 83% yield. In the case of N⁶N⁶-diBoc-deazaadenine (15b), however, the selectivity toward the N₉-isomer (17b) over the N₇-isomer (16b) increased to 10:1 in 88% yield, The major product 17b was easily separated from the reaction mixture by silica-gel column chromatography. The removal of Boc, trityl and isopropylindenyl groups of 17b by methanolic HCl gave 3-deazaneplanocin A (2) in 65% overall yield from 6-chloro-3-deazaadenine (14) (7 steps). The regioselectivity of 15b toward the N₉-position in the Mitsunobu reaction was enhanced to 5- and 2.5-fold compared with those of 5 and 15a, respectively. Thus, N⁶N⁶-diBoc-3-deazaadenine (15b) was successfully applied to the preparative scale synthesis of 3-deazaneplanocin A (2) in 65% overall yield from 7.

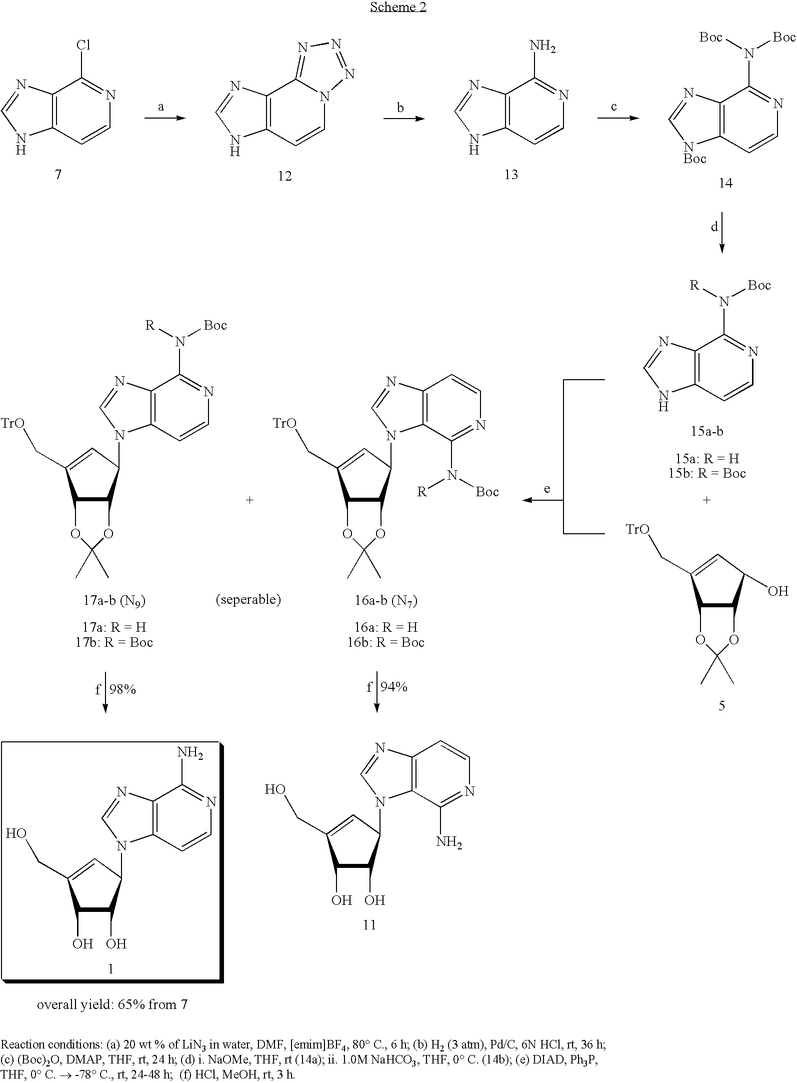

TABLE 2
Results of Mitsunobu reaction and overall yield

| | | Ratio of N₉:N₇ | Overall % yield |
|---|---|---|---|
| Entry | Compound | (yield^{a}, %) | of 2 from 7 |
| 1 | 7 | 2:1 (94)^{b} | 10 |
| 2 | R = H (15a) | 4:1 (83)^{b} | 20 |
| 3 | R = Boc (15b) | 10:1 (88)^{c} | 65 |

^{a}Yield of couling reaction.
^{b}Ratio determined by ¹H-NMR.
^{c}Ratio isolated by silica gel column.

On the basis of the Mitsunobu reaction of 5 with 7 or 15a-b, the condensation reaction of 5 with imidazol derivative (19) or a protected 3-deazaguanine (23) was initially investigated with the Mitsunobu reaction in order to synthesize carbocyclic 3-deazaguanosine (4). The coupling reaction of 5 with 19, which was prepared from 18 by following the previously reported method,¹⁶ provided N₇-isomer (20) in 91% yield using the condition of Ph₃P and DIAD in THF. The precursor 20 was subsequently converted to carbocyclic N₇-3-deazaguanosine (21) in 56% yield (2 steps) by using methamolic ammonia and hydrogen chloride. The structure of 21 was determined by ¹H- and ¹³C-NMR, as well as UV data (λmax 261 and 317 nm, pH 7.0), which was compared with previously reported data for N₇-3-deazaguanosine (3).^{16b} Furthermore, the Mitsunobu reaction of 5 with a protected 3-deazaguanine (23), which was synthesized from 19 by using cyclization with ammonia in a steel bomb and protection with isobutanoic anhydride, afforded a mixture of N₉/N₇-isomer (ratio 1:1) in 20% yield along with several by-products. Therefore, we investigated a more efficient condensation reaction in order to enhance the amount of N₉-isomer and reaction yield. The hydroxy group of 5 was transformed to a methansufonyl (Ms) group with MsCl in 92% yield.¹⁰ The substitution reaction of 22 with 23 in the presence of NaH and DMF solution gave N₉-isomer (24) in 40% yield along with 5% of N₇-isomer (25), which could easily be removed from 24 by using silica gel column chromatography. The structure of 24 was determined with 1-D NOE and ¹H- and ¹³C-NMR, as well as UV data ((λmax 271 and 300 nm, pH 7.0). All data was consistent with 3-deazaguanosin (3) from previously reported literatures.^{16b} The significant interaction between the C₁′—H and aromatic C₃—H of 24 was observed in NOE experiment as shown in FIG. 2. Subsequently, the compound 24 was treated with methalolic ammonia, followed by aqueous 0.1 M HCl solution to afford carbocyclic 3-deazaguanosine (C-3-DG, 4) in an 11% yield from imidazole derivative (19). In the case of the synthesis of C-3-DG (4), this simple substitution reaction was a more convenient strategy than the Mitsunobu reaction both by regioselectivity (N₉) and overall yield.

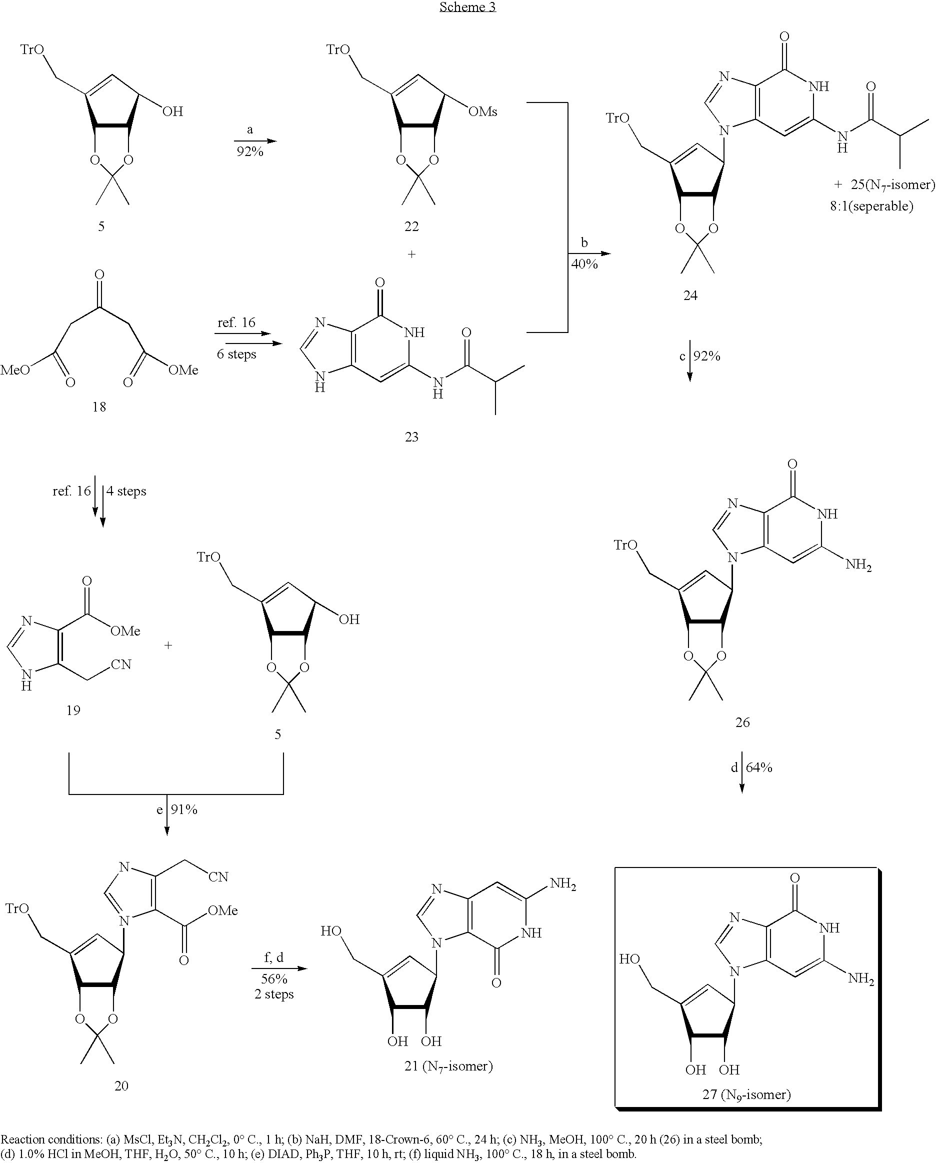

### Biological Data

### Antiviral Data

Various compounds according to the present invention were tested in vitro to determine biological activity. The following tables present the biological data for the indicated compounds.

### Anti-HBV Activity

In the first biological experiment, a number of 7-deazaneplanocin compounds were tested in vitro to determine activity against hepatitis B virus (HBV). The results of that experimental testing is provided in the following table.

TABLE
In vitro activity and toxicity data of 7-deazaneplanocin
analogs against Hepatitis B Virus

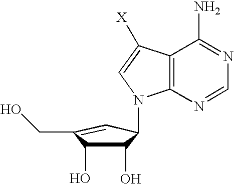

| | | EC₅₀ | EC₉₀ | CC₅₀ | |
|---|---|---|---|---|---|
| X | Assay | (μM) | (μM) | (μM) | SI |
| H | | | | | |
| F | VIR | >10 | >10 | >300 | |
| Cl | VIR | >10 | >10 | >300 | |
| Br | VIR | >10 | >10 | >300 | |
| I | VIR | 0.431 | 1.8 | >300 | >167 |
| | RI | 1.6 | 5.9 | >300 | >51 |
| CN | VIR | >10 | >10 | >300 | |
| NO₂ | VIR | >10 | >10 | >300 | |
| vinyl | VIR | 3.3 | 9.1 | >300 | >33 |
| | RI | 10 | 26 | >300 | >11 |
| acetylenyl | VIR | 0.315 | 1.6 | >300 | >188 |
| | RI | 1.8 | 7.9 | >300 | >40 |
| CONH₂ | VIR | >10 | >10 | >300 | |

VIR data are based on extracellular virion HBV DNA
RI data are based on intracellular HBV DNA replication intermediates
SI = CC₅₀/EC₉₀

HBV DR Assay Against Various Drug Resistant Strains of HBV

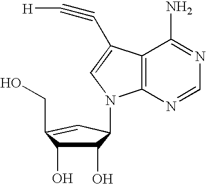

| | | | | | | Control: | Control: |
|---|---|---|---|---|---|---|---|
| | | | | Control: | Control: | ADV | ADV |
| | | | | 3TC(um) | 3TC(um) | (um) | (um) |
| Strain | Units | EC50 | EC90 | EC50 | EC90 | EC50 | EC90 |
| L180M | uM | 2 | 8.2 | 10 | F31 | 1.6 | 6.8 |
| LM/MV | uM | 2.8 | 9 | >100 | >100 | 1.2 | 6.1 |
| M2041 | uM | 3 | 9.5 | >100 | >100 | 1.8 | 7 |
| M204V | uM | 2.1 | 8.9 | >100 | >100 | 1.5 | 6.3 |
| N236T | uM | 5.6 | 16 | 0.3 | 0.8 | 7.7 | 30 |
| WT | uM | 2.5 | 8.7 | 0.2 | 0.6 | 1.3 | 6 |

Comments: Test acetyleneic compound was active against all tested lamivudine and adefovir dipovoxil resistant mutants. Activity against the ADV-resistant mutant, N236T, appeared to be slightly reduced.

### Anti-HCV Activity

A number of a number of 7-deazaneplanocin compounds were tested in vitro to determine activity against hepatitis C virus (HCV). The results of that experimental testing is provided in the following table.

TABLE
Anti-HCV data for 7-deazaneplanocin analogs

| | | Activity | | | | | |
|---|---|---|---|---|---|---|---|
| | | (% | | | | | |
| | | inhibition | Cytotoxicity | | | | |
| Compound | | virus | (% cell | EC₅₀ | IC₅₀ | SI | |
| Structure | Assay type | control) | control) | (μM) | (μM) | value | Comments |
| 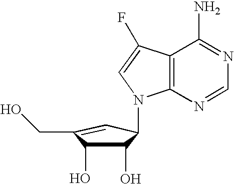 | HCV RNA replicon | 9.5 | 112.7 | Inactive in primary, single-dilution test | | | |
| 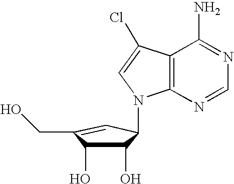 | HCV RNA replicon | 70.6 | 81.2 | 14.7 | 50.09 | >3.48 | HT @ 100 μM |
| 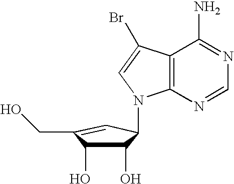 | HCV RNA replicon | 80.9 | 76.3 | 10.5 16.7 | >20 41.72 | >1.91 2.5 | HT @ 20 μM HT @ 100 μM |
| 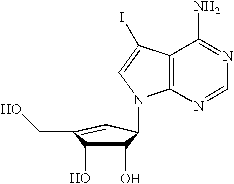 | HCV RNA replicon | 89.7 | 35.2 | 2.14 3.07 | 14.96 >5 | >6.99 >1.63 | HT @ 100 μM HT @ 5 μM |
| 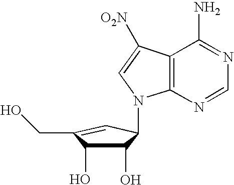 | HCV RNA replicon | 31.5 | 101.5 | NA | NA | NA | <1 |
| 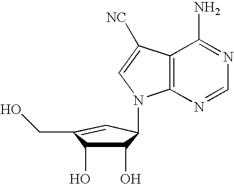 | HCV RNA replicon | 0.5 | 103.3 | Inactive in primary, single-dilution test | | | |
| 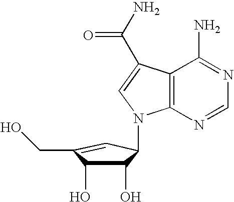 | HCV RNA replicon | 93.5 | 44.2 | 1.85 | 11.8 | 6.38 | <1 |
| 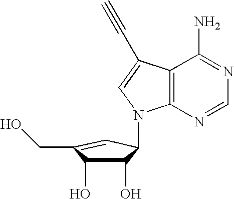 | HCV RNA replicon | 73.3 | 86.6 | 12.3 20.1 | >20 48.94 | >1.63 2.44 | HT @ 20 μM HT @ 100 μM |
| 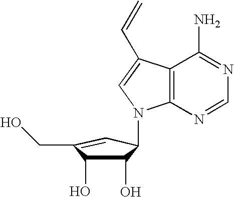 | HCV RNA replicon | 49.2 | 99.7 | NA | NA | NA | <1 |

| | | | | |
|---|---|---|---|---|
| 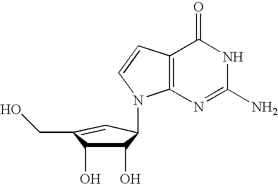 | HCV RNA replicon | 10.1 | 99.8 | Inactive in primary, single-dilution test |

### Anti-Viral Activity of 3-Deazaneplanocin A (3-DNPA)

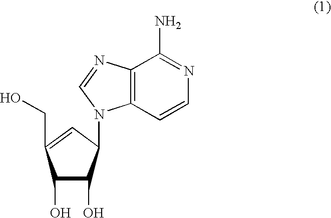

The 3-DNPA (1) has been evaluated for its antiviral activity against hepatitis C virus (HCV), hepatitis B virus (HBV), respiratory syncytial virus A (RSV A), Rhinovirus, parainfluenza virus (PIV), measles, Flu A (H1N1 and H3N3), adenovirus, cowpox, yellow fever, severe acute respiratory syndrome (SARS), vaccinia, herpes simplex virus type 1 and 2 (HSV-1 and HSV-2), varicella zoster virus (VZV) and human cytomegalovirus (HCMV) as well as for its cytotoxicity. The results of the antiviral activity against various viruses are summarized in table 3. Prepared 3-DNPA (1) exhibited significantly potent antiviral activity against measles (EC₅₀ 0.4 μM), HBV (EC₅₀ 0.59 μM) and HCMV (EC₅₀ 0.36 μM), respectively. Also, 3-DNPA (1) showed moderate antiviral activity against vaccinia (EC₅₀ 2.9 μM), HCV (EC₅₀ 1.44 μM), Rhinovirus (EC₅₀ 4.0 μM), RSV A (EC₅₀ 4.0 μM), HSV-2 (EC₅₀ 43.5 μM) and Adeno virus (EC₅₀ 17.0 μM), respectively. However, 3-DNPA (1) did not show any significant antiviral activity against the other viruses including SARS (EC₅₀>100 μM), PIV (EC₅₀>100 μM), Flu A (EC₅₀>100 μM), yellow fever (EC₅₀>100 μM), HSV-1 (EC₅₀>300 μM) and VZV (EC₅₀>300 μM), respectively. N₇-3-Deazaneplanocin A (11) was showed moderate antiviral activity against only Flu A (H5N1) (EC₅₀ 32 μM) and Flu B (EC₅₀ 8 μM) among above referred viruses.

TABLE 3
Antiviral activity of 3-DNPA, N7-3-DNPA and 3-DG against various viruses

| | | | | | | N₇-3- | | C-3- | |
|---|---|---|---|---|---|---|---|---|---|
| | | | | 3-DNPA (2) | | DNPA (11) | | DG (4) | |
| | | | | EC₅₀ | | EC₅₀ | | EC₅₀ | |
| Entry | Virus | Assay | Cell line | (μM) | SI | (μM) | SI | (μM) | SI |
| 1 | SARS | NR | Vero 76 | >100 | 0.7 | >100 | >100 | — | — |
| | | Visual | | >100 | 10 | >100 | 51 | — | — |
| 2 | RSV A | NR | MA-104 | 4.0 | >100 | — | — | — | — |
| | | Visual | | 100 | >100 | — | — | — | — |
| 3 | RV | NR | HO-1 | 4.0 | 4 | — | — | — | — |
| | | Visual | | 32 | 32 | — | — | — | — |
| 4 | PIV | NR | MA-104 | >100 | >100 | — | — | — | — |
| | | Visual | | >100 | >100 | — | — | — | — |
| 5 | Measles | NR | CV-1 | 0.4 | 12 | — | — | — | — |
| | | Visual | | 0.9 | 10 | — | — | — | — |
| 6 | Flu A | NR | MDCK | >25 | 25 | >32 | 32 | — | — |
| | (H1N1) | Visual | | >100 | >100 | >32 | 32 | — | — |
| 7 | Flu A | NR | MDCK | >100 | >100 | >32 | 32 | — | — |
| | (H3N2) | Visual | | >100 | >100 | >32 | 32 | — | — |
| 8 | Flu A | NR | MDCK | >100 | >100 | 29 | >100 | — | — |
| | (H5N1) | Visual | | >100 | >100 | 32 | >100 | — | — |
| 9 | Flu B | NR | MDCK | — | — | 34 | >100 | — | — |
| | | Visual | | — | — | 8 | >100 | — | — |
| 10 | Adeno | NR | A-549 | 17.0 | 17 | — | — | — | — |
| | | Visual | | 24.0 | 24 | — | — | — | — |
| 11 | YF | NR | Vero | >100 | >100 | >100 | >100 | — | — |
| | | Visual | | >100 | >100 | >100 | >100 | — | — |
| 12 | Cowpox | CPE | HFF | 182 | >300 | 273 | >300 | — | — |
| | | | Cells | | | | | | |
| 13 | VV | CPE | HFF | 2.9 | >300 | >300 | >300 | — | — |
| | | | Cells | | | | | | |
| 14 | HSV-1 | CPE | HFF | >300 | >300 | — | — | — | — |
| | | | Cells | | | | | | |
| 15 | HSV-2 | CPE | HFF | 43.5 | >300 | — | — | — | — |
| | | | Cells | | | | | | |
| 16 | HCMV | CPE | HFF | 0.36 | 183 | — | — | — | — |
| | | | Cells | | | | | | |
| 17 | VZV | CPE | HFF | >300 | >300 | — | — | — | — |
| | | | Cells | | | | | | |
| 18 | HCV | HRR | Huh7 ET | 1.44 | 0.89 | — | — | — | — |
| 19 | HBV | VIR | Huh7 ET | 0.59 | 178 | >10 | >300 | — | — |
| 20 | Tacaribe | NR | Vero 76 | — | — | 85 | 47 | — | — |
| | | Visual | | — | — | 52 | 52 | — | — |
| 21 | Dengue | NR | Vero | — | — | >100 | >100 | — | — |
| | | Visual | | — | — | >100 | >100 | — | — |
| 22 | VEE | NR | Vero | — | — | 84.6 | >100 | — | — |
| | | Visual | | — | — | >100 | >100 | — | — |
| 23 | RVF | NR | Vero 76 | — | — | >100 | >100 | — | — |
| | | Visual | | — | — | >100 | 32 | — | — |

RV = Rhinovirus,
YF = Yellow Fever,
VV = Vaccinia virus,
NR = Neutral Red,
HO-1 = HeLa Ohio-1,
HRR = HCV RNA replicon,
SI = CC₅₀/IC₅₀

### EXAMPLES

### Experimental Section for Novel Cyclopentenoside Compounds (5-Membered Rings)

NMR spectra were recorded on 400 and 500 MHz Fourier transform spectrometer; chemical shifts are reported in parts per million (δ), and signals are quoted as s (singlet), d (doublet), t (triplet), q (quartet), m (multiplet), br (broad), dd (double of doublets) and dt (double of triplets). Optical rotations were measured by a Jasco DIP-370 digital polarimeter. High-resolution mass spectra were recorded on a Micromass Autospec high-resolution mass spectrometer. TLC was performed on 0.25 mm silica gel. Purifications were carried out using silica gel (60 Å, 32-63 mm). The data of elemental analysis were provided by Atlantic Microlab Inc., Norcross, Ga.

### Scheme 1, FIG. 1

### 6-Hydroxymethyl-2,2-dimethyl-tetrahydro-furo[3,4-d][1,3]dioxol-4-ol (5)

To a solution of D-ribose (50.0 g, 0.34 mol) and catalytic amounts of TsOH.H₂O (1.90 g, 1.0 mmol) in 500 mL of acetone, 2,2-dimethoxypropane (38.16 g, 0.37 mol) was added at 0° C. The suspension was stirred for ca. 1 hr at room temperature until a clean solution was achieved. The solution was then treated with NaHCO₃ (0.10 g, 1.20 mmol) and was stirred for additional 30 min at room temperature. The solid was filtered and the filtrate was adsorbed on silica gel and purified by silica gel column chromatography (hexae:EtOAc=3:1to 1:1 v/v) to give compound 5 (54.0 g, 0.29 mol) in 90% yield. R_{f}=0.2 (hexane:EtOAc=3:1 v/v); ¹H-NMR (CDCl₃, 500 MHz) δ 5.65 (d, J=6.0, 0.9H), 5.35 (d, J=6.0, 0.9H), 4.75 (d, J=6.0, 0.9H), 4.52 (d, J=6.0, 0.9H), 4.33 (t, J=2.5, 0.9H), 4.30 (br, 0.9H), 3.65 (t, J=12.0, 1.8H), 1.43 (s, 2.7H), 1.27 (s, 2.7H); ¹³C-NMR (CDCl₃, 125 MHz) δ 112.13, 102.65, 87.56, 86.66, 81.60, 63.41, 26.30, 24.66; α-form: ¹H-NMR (CDCl₃, 500 MHz) δ 5.38 (dd, J=12.0, 8.0, 0.1H), 4.87 (t, J=10.0, 0.1H), 4.66 (dd, J=14.0, 6.0, 0.11H), 4.59 (dd, J=14.0, 8.0, 0.1H), 4.35 (m, 0.1H), 4.12 (dd, J=10.0, 6.0, 0.1H1), 3.67 (br, 0.2H), 1.52 (s, 0.3H), 1.34 (s, 0.3H); ¹³C-NMR (CDCl₃, 125 MHz) δ 114.12, 89.01, 81.47, 81.09, 79.42, 63.08, 26.70, 25.48.

### 2,2-Dimethyl-6-trityloxymethyl-tetrahydro-furo[3,4-d][1,3]dioxol-4-ol (6a)

To a solution of compound 5 (15.30 g, 80.46 mmol), catalytic amount of DMAP (0.30 g, 2.41 mmol) and trityl chloride (26.92 g, 96.56 mmol) in 200 mL of anhydrous DMF, Et₃N (12.21 g, 0.12 mol) were added at room temperature under nitrogen atmosphere. The resulting solution was stirred for 48 hr at room temperature and poured into ice water (100 mL). The organic layer was extracted with CH₂Cl₂ (200 mL×3), washed with saturated aqueous NH₄Cl (100 mL×2) and water (200 mL), and then dried over Na₂SO₄. The solvent was removed under reduced pressure and the residue was purified by silica gel column chromatography (hexane:EtOAc=10:1 to 2:1 v/v) to give compound 6a (25.0 g, 58.0 mmol) in 85% yield. R_{f}=0.2 (hexane:EtOAc=10:1 v/v); ¹H-NMR (CDCl₃, 500 MHz) δ 7.41-7.38 (m, 6H), 7.34-7.24 (m, 12H), 5.33 (d, J=8.5, 0.8H) 4.79 (d, J=6.0, 0.8H), 4.66 (d, J=6.0, 0.8H), 4.35 (t, J=4.0, 0.8H), 3.95 (d, J=9.0, 0.8H), 3.42 (dd, J=10.0, 4.0, 0.8H), 3.34 (dd, J=10.0, 4.0, 0.8H), 1.48 (s, 2.4H), 1.34 (s, 2.4H); ¹³C-NMR (CDCl₃, 125 MHz) δ 142.83, 128.71, 128.15, 127.54, 112.29, 103.57, 88.25, 87.11, 86.10, 81.96, 65.10, 26.56, 25.14; α-form: ¹H-NMR (CDCl₃, 500 MHz) δ 7.41-7.38 (m, 1.2H), 7.34-7.24 (m, 2.4H), 5.75 (dd, J=11.5, 4.0, 0.2H), 4.74 (dd, J=11.5, 4.0, 0.2H), 4.58 (d, J=6.5, 0.2H), 4.19 (t, J=3.0, 0.2H), 4.01 (d, J=11.5, 0.2H), 3.45 (dd, J=10.0, 3.0, 0.2H), 3.01 (d, J=10.0, 3.0, 0.2H), 1.55 (s, 0.6H), 1.37 (s, 0.6H); ¹³C-NMR (CDCl₃, 125 MHz) δ 143.52, 128.62, 128.03, 127.26, 113.01, 98.02, 87.60, 82.23, 80.15, 79.59, 65.51, 26.21, 24.79; HRMS (ES) calcd for C₂₇H₂₈O₅ (M+Na⁺) 455.1835, found 455.1877.

### 1-[5-((1-Hydroxy-2-trityloxy-ethyl)-2,2-dimethyl-[1,3]dioxolan-4-yl]-prop-2-en-1-ol (7a)

To a solution of compound 6a (23.76 g, 54.94 mmol) in 300 mL of anhydrous THF, 165.0 mL of vinylmagnesium bromide (3.0 equiv., 1.0 M of THF) was added at −78° C. under nitrogen atmosphere. After 1 hr, the temperature was raised to room temperature and the reaction mixture was stirred for additional 6 hr. which was treated with saturated NH₄Cl solution (100 mL) dropwise at 0° C., and the resulting solution was poured into iced ether-saturated aqueous NH₄Cl solution (400 mL, 3:1 v/v). The organic layer was separated and the aqueous layer was washed with ether (100 mL×2). The combined organic layer was dried over MgSO₄, filtered and concentrated in vaccuo. The residue was purified on a silica gel column (hexane:EtOAc=10:1 to 3:1 v/v) to give compound 7a (25.29 g, 54.90 mmol) in 100% yield. R_{f}=0.2 (hexane:EtOAc=10:1 v/v); [α]²⁷_{D} +12.32 (c 0.50, CHCl₃); ¹H-NMR (CDCl₃, 500 MHz) δ 7.56 (d, J=8.0, 6H), 7.38 (m, 10H), 6.12 (ddd, J=17.0, 10.0, 5.0, 1H) 5.52 (d, J=17.0, 1H), 5.34 (d, J=10.5, 1H), 4.41 (s, 2H), 4.22 (dd, J=10.0, 5.0, 1H), 4.13 (dd, J=10.0, 5.0, 1H), 4.04 (m, 1H), 3.65 (d, J=3.5, 2H), 3.61 (dd, J=10.0, 3.5, 1H), 3.40 (dd, J=10.0, 7.5, 1H), 1.37 (s, 3H), 1.36 (s, 3H); ¹³C-NMR (CDCl₃, 125 MHz) δ 143.83, 137.61, 128.74, 128.03, 127.28, 116.37, 108.89, 87.15, 80.76, 77.42, 69.96, 69.08, 65.24, 28.07, 25.63; HRMS (ES) calcd for C₂₉H₃₂O₅ (M+Na⁺) 483.2148, found 483.2168; Anal. Calcd for C₂₉H₃₂O₅: C, 75.63; H, 7.00. Found: C, 75.47; H, 7.00.

### 1-{5-[1-(tert-Butyl-dimethyl-silanyloxyl)-allyl]-2,2-dimethyl-[1,3]dioxolan-4-yl}-2-trityloxy-ethanol (8a)

To a solution of compound 7a (25.29 g, 54.90 mmol) in 300 mL of anhydrous CH₂Cl₂-DMF solution (10:1 v/v), imidazole (11.23 g, 16.50 mmol) and TBDMSCl (10.34 g, 68.64 mmol) were added at 0° C. under nitrogen atmosphere. The reaction mixture was stirred for 24 hr at room temperature and then poured into 500 mL of a mixture of solvent (ether-water, 1:1 v/v). The organic layer was separated and the aqueous layer was washed with ether (100 mL×2). The combined organic layer was dried over MgSO₄, filtered and concentrated. The residue was purified on a silica gel column (hexane:EtOAc=30:1 v/v) to give compound 8a (26.10 g, 45.02 mmol) in 82% yield. R_{f}=0.2 (hexane:EtOAc=30:1 v/v); ¹H-NMR (CDCl₃, 500 MHz) δ 7.53-7.47 (m, 6H), 7.34-7.23 (m, 9H), 5.93 (m, 1H) 5.35-5.22 (m, 2H), 4.51 (s, 0.8H), 4.34 (m, 0.2H), 4.28 (m, 0.4H), 4.14 (m, 2H), 4.07 (m, 1.6H), 3.74 (s, 0.8H), 3.68 (m, 0.2H), 3.44 (m, 1.2H), 3.25 (m, 0.8H), 1.40 (s, 0.6H), 1.32 (s, 0.6H), 1.29 (s, 2.4H), 1.27 (s, 2.4H), 0.96 (s, 7.2H), 0.86 (s, 1.8H), 0.18 (s, 2.4H), 0.12 (s, 2.4H), 0.11 (s, 0.6H), 0.03 (s, 0.6H); ¹³C-NMR (CDCl₃, 125 MHz) δ 149.24, 148.64, 142.77, 133.81, 133.68, 132.90, 132.82, 132.64, 132.11, 131.77, 122.95, 120.80, 112.93, 112.13, 92.15, 91.30, 84.95, 82.99, 78.56, 76.31, 74.33, 73.89, 70.32, 70.14, 32.98, 32.77, 30.90, 30.70, 23.22, 23.05, 1.08, 0.60, 0.26, 0.00; HRMS (ES) calcd for C₃₅H₄₆O₅Si (M+Na⁺) 597.3013, found 597.3152.

### 1-{5-[1-(tert-Butyl-dimethyl-silanyloxyl)-allyl]-2,2-dimethyl-[1,3]dioxolan-4-yl}-2-trityloxy-ethanone (9a)

To a solution of oxalyl chloride (3.59 g, 41.02 mmol) in 100 mL of anhydrous CH₂Cl₂, DMSO (5.82 g, 82.05 mmol) was added at −60° C. under nitrogen atmosphere and then the resulting solution was stirred 10 min. A solution of compound 8a (19.0 g, 32.82 mmol) in 200 mL of anhydrous CH₂Cl₂ was added to the reaction mixture dropwise over 20 min at −60° C. After 30 min. Et₃N (16.60 g, 164.09 mmol) was added dropwise over 20 min to the reaction mixture at −60° C. The mixture was stirred for 1 hr at −60° C. and then stirred for 30 min at room temperature. The reaction mixture was treated with 200 mL of water dropwise at 0° C. The organic layer was separated and the aqueous layer was extracted with CH₂Cl₂ (200 mL×3). The combined organic layer was washed with brine (200 mL×2), dried over MgSO₄, filtered and concentrated under reduced pressure. The residue was purified on a silica gel column (hexane:EtOAc=20:1 to 10:1 v/v) to give compound 9a (18.0 g, 31.64 mmol) in 95% yield. R_{f}=0.5 (hexane:EtOAc=20:1 v/v); [α]²³_{D} −14.97 (c 1.00, CHCl₃); ¹H-NMR (CDCl₃, 500 MHz) δ 7.57 (m, 6H), 7.39-7.29 (m, 9H), 5.90 (m, 1H) 4.64 (d, J=7.5, 1H), 4.41 (m, 2H), 4.24 (d, J=18.0, 1H), 3.95 (d, J=18.0, 1H), 1.42 (s, 3H), 0.90 (s, 9H), 0.08 (s, 3H), 0.05 (s, 3H); ¹³C-NMR (CDCl₃, 125 MHz) δ 148.13, 141.87, 133.31, 133.25, 132.49, 131.69, 122.50, 113.95, 91.71, 87.01, 84.01, 77.67, 74.06, 30.78, 30.63, 29.29, 22.95; HRMS (ES) calcd for C₃₅H₄₄O₅Si (M+Na⁺) 595.2856, found 595.2873.

### tert-Butyl-{1l-[2,2-dimethyl-5-(1-trityloxymethyl-vinyl)-[1,3]dioxolan-4-yl]-allyloxy}-dimethyl-silane (10a)

To a suspension of Ph₃PCH₃Br (52.63 g, 147.33 mmol) in 100 mL of THF, n-BuLi (82.85 mL, 1.6 M in hexane) was added at 0° C. under N₂ atmosphere. After 30 min, a solution of compound 9a (17.0 g, 29.46 mmol) in 200 mL of THF was added to the reaction mixture at 0° C. The resulting mixture was stirred for 12 h at room temperature, then treated with 50 mL of MeOH and 100 mL of water and then poured into 300 mL of ether-water solution (2:1 v/v). The organic layer was separated and the aqueous layer was extracted with ether (200 mL×2). The combined organic layer was washed with brine (20 mL×2), dried over MgSO₄, filtered and concentrated in vacuo. The residue was purified on a silica gel column (hexane:EtOAc=50:1 to 10:1 v/v) to give compound 10a (16.02 g, 27.99 mmol) in 95% yield. R_{f}=0.65 (hexane:EtOAc=10:1 v/v); ¹H-NMR (CDCl₃, 500 MHz) δ 7.54 (m, 6H), 7.34-7.30 (m, 9H), 5.74 (m, 1H) 5.72 (s, 1H), 5.46 (s, 1H), 5.24 (d, J=10.0, 1H), 5.06 (d, J=17.5, 1H), 4.82 (d, J=5.5, 1H), 3.98 (m, 2H), 3.79 (d, J=13.5, 1H), 3.60 (d, J=13.5, 1H), 1.36 (s, 3H), 1.32 (s, 3H), 0.89 (s, 9H), 0.01 (s, 6H); ¹³C-NMR(CDCl₃, 125 MHz) δ 148.25, 146.08, 142.74, 132.73, 131.98, 131.15, 121.04, 117.65, 112.04, 90.88, 84.73, 82.92, 77.29, 68.84, 30.57, 30.22, 29.15, 22.27, 0.71, 0.01; HRMS (ES) calcd for C₃₆H₄₆O₄Si (M+Na⁺) 593.3063, found 593.3146.

### 1-[2,2-Dimethyl-5-(1-trityloxymethyl-vinyl)-[1,3]dioxolan-4-yl]-prop-2-en-)-ol (11a)

The solution of compound 10a (16.0 g, 27.99 mmol) in 100 mL of THF was treated TBAF (40 mL, 1.0 M in THF) at room temperature. After stirring for 2 hr, the reaction mixture was adsorbed on silica gel and purified on a silica gel column (hexane:EtOAc=30:1 v/v) to give compound 11a (12.52 g, 27.43 mmol) in 98% yield. R_{f}=0.3 (Hex:EtOAc=10:1 v/v); ¹H-NMR (CDCl₃, 500 M ) δ 7.46 (m, 6H), 7.32-7.22 (m, 9H), 5.94 (m, 1H) 5.61 (s, 1H), 5.51 (s, 1H), 5.27 (dt, J=17.0, 1.5, 1H), 5.18 (dt, J=11.0, 1.5, 1H), 4.58 (d, J=6.0, 1H), 3.96 (m, 2H), 3.89 (dd, J=8.0, 6.0, 1H), 3.74 (dd, J=24.0, 13.0, 2H), 2.22 (d, J=4.0, 3H), 1.37 (s, 3H), 1.29 (s, 3H); ¹³C-NMR (CDCl₃, 125 MHz) δ 143.73, 142.22, 137.73, 128.71, 127.99, 127.25, 116.13, 113.96, 108.23, 87.55, 80.59, 77.95, 70.34, 65.42, 27.18, 25.19; HRMS (ES) calcd for C₃₀H₃₂O₄ (M+Na⁺) 479.2199, found 479.2299.

### 2,2-Dimethyl-6-trityloxymethyl-4,6a-dihydro-3aH-cyclopenta[1,3]dioxol-4-ol ((+)-12a)

Method A: To a solution of compound 11a (10 g, 21.90 mmol) of 400 mL of anhydrous CH₂Cl₂, 2^{nd}-generation Grubbs catalyst (0.4 g, 0.44 mmol) was added at room temperature under argon atmosphere. After stirring for 24 h, the reaction mixture was adsorbed on silica gel and purified on a silica gel column (hexane:EtOAc=10:1 to 5:1 v/v) to give compound (+)-12a (8.82 g, 1.86 mmol) in 94% yield. R_{f}=0.2 (hexane:EtOAc=5:1 v/v); [α]²³_{D} +33.21 (c 1.00, CHCl₃); ¹H-NMR(CDCl₃, 500 MHz) δ 7.46 (m, 6H), 7.29-7.20 (m, 9H), 5.99 (s, 1H) 5.23 (s, 1H), 4.86 (d, J=5.0, 1H), 4.73 (t, J=5.0, 1H), 4.57 (m, 1H), 3.88 (d, J=15.0, 1H), 3.67 (d, J=15.0, 1H), 2.76 (d, J=10.0, 1H), 1.36 (s, 3H), 1.35 (s, 3H); ¹³C-NMR (CDCl₃, 125 MHz) δ 143.95, 143.43, 129.84, 128.63, 127.94, 127.15, 112.52, 87.01, 83.36, 77.93, 73.51, 60.95, 27.83, 26.93; HRMS (ES) calcd for C₂₈H₂₈O₄ (M+Na⁺) 451.1886, found 451.1870.

Method B: To a solution of compound 14 (1.0 g, 5.37 mmol), catalytic amount of DMAP (0.07 g, 0.54 mmol) and trityl chloride (1.90 g, 6.71 mmol) in 20 mL of anhydrous DCM, Et₃N (1.0 mL, 6.71 mmol) was added at room temperature under N₂ atmosphere. After 12 hr at room temperature, the reaction mixture was poured into ice water (20 mL). The product was extracted with CH₂Cl₂ (20 in L×3) from aqueous layer. The combined solution washed with saturated aqueous NH₄Cl (10 mL×2), water (20 mL) and brine (10 mL×2) and then dried over Na₂SO₄, filtered, concentrated in vacuo. The residue was purified by silica gel column chromatography (hexane:EtOAc=20:1 to 4:1 v/v) to give compound (+)-12a (2.21 g, 4.94 mmol) in 92% yield. [α]²²_{D} +30.57 (c 0.57, CHCl₃).

### 2,2-Dimethyl-6-trityloxymethyl-4,6a-dihydro-3aH-cyclopenta[1,3]dioxol-4-ol ((+)-12b)

To a solution of Ph₃P (0.15 g, 1.26 mmol) and DIAD (0.26 g, 1.26 mmol) in 5.0 mL of anhydrous THF, benzoic acid (0.15 g, 1.26 mmol) and a solution of compound (+)-12a (0.36 g, 0.84 mmol) in 10.0 mL of anhydrous THF were added at 0° C. under N₂ atmosphere. After the suspension overnight at room temperature, the reaction mixture was adsorbed on silica gel and purified on silica gel pad (hexane:EtOAc=1:2 v/v) to give a crude product with a little amount of DIAD. The crude intermediate was treated with LiOH (0.11 g, 2.52 mmol) in 20 mL of ThF—H₂O solution (3:1 v/v) for 12 hr at room temperature. The basic solution was neutralized by addition of 1.0 M HCl solution. The product was extracted with ethyl acetate (3×20 mL) from aqueous layer. The combined organic layer was washed with brine (20 mL), dried over MgSO₄, filtered and concentrated under reduced pressure. The residue was purified by silica gel column chromatography (hexane:EtOAc=10:1 to 4:1 v/v) to give compound (+)-12b (0.30 g, 0.71 mmol) in 84% yield (2 steps). [α]²⁴_{D} +2.44 (c 0.50, CHCl₃); ¹H-NMR (CDCl₃, 500 MHz) δ 7.46 (m, 6H), 7.29-7.24 (m, 9H), 5.99 (s, 1H) 5.08 (s, 1H), 4.76 (s, 1H), 4.51 (s, 1H), 3.89 (d, J=15.0, 1H), 3.71 (d, J=15.0, 1H), 2.03 (br, 1H), 1.33 (s, 3H), 1.30 (s, 3H); ¹³C-NMR (CDCl₃, 125 MHz) δ 147.26, 143.94, 128.61, 127.88, 127.28, 127.08, 111.89, 86.47, 83.80, 80.01, 70.06, 61.34, 27.49, 26.17; HRMS (ES) calcd for C₂₇H₂₈O₅ (M+Na⁺) 451.1886, found 451.1870.

### 6-(tert-Butyl-diphenyl-silanyloxymethyl)-2,2-dimethyl-tetrahydro-furo[3,4-d][1,3]dioxol-4-ol (6b)

To a solution of compound 5 (19.0 g, 99.92 mmol) in 200 mL of anhydrous CH₂Cl₂, TBDPSCl (25.60 g, 99.92 mmol) and imidazole (20.21 g, 149.88 mol) were added at 0° C. under nitrogen atmosphere. The suspension solution was allowed to stir for 24 hr at room temperature. The reaction mixture was absorbed on silica gel and then purified by silica gel column chromatography (hexane:EtOAc=20:1 v/v) to give compound 6b (40.0 g, 88.20 mmol) in 88% yield. R_{f}=0.6 (hexane:EtOAc=20:1 v/v); ¹H-NMR (CDCl₃, 500 MHz) δ 7.66 (m, 4H), 7.47-7.40 (m, 6H), 5.35 (d, J=8.0, 1H), 4.72 (m, 1H), 4.61 (m, 1H), 4.55 (d, J=10.0, 1H), 4.28 (s, 1H), 3.82 (d, J=11.0, 1H), 3.65 (d, J=11.0, 1H), 1.47 (s, 3H), 1.31 (s, 3H), 1.09 (s, 9H); ¹³C-NMR (CDCl₃, 125 MHz) δ 135.77, 135.60, 130.43, 130.25, 128.14, 128.08, 127.95, 112.15, 103.41, 87.31, 87.09, 81.74, 65.53, 26.95, 26.91, 26.52, 25.02, 19.18; α-isomer: δ 7.66 (m, 0.8H), 7.47-7.40 (m, 1.2H), 5.62 (d, J=11.0, 0.2H), 4.78 (m, 0.2H), 4.66 (m, 0.2H), 4.15 (s, 0.2H), 4.11 (m, 0.4H), 3.98 (d, J=11.0, 0.2H), 3.82 (m, 0.2H), 3.61 (m, 0.2H), 1.55 (s, 0.6H), 1.39 (s, 0.6H), 1.05 (s, 1.8H); HRMS (ES) calcd for C₂₄H₃₂O₅Si (M+Na⁺) 451.1917, found 451.1924.

### 1-{5-[2-(tert-Butyl-diphenyl-silanyloxy)-1-hydroxy-ethyl]-2,2-dimethyl-[1,3]dioxolan-4-yl}-prop-2-en-1-ol (7b)

To a solution of compound 6b (36.0 g, 78.90 mmol) in 400 mL of anhydrous THF, 200 mL of vinylmagnesium bromide (3.0 equiv., 1.0 M of THF) was added at −78° C. under nitrogen atmosphere. After 1 hr, the temperature was raised to room temperature and the reaction mixture was stirred for additional 6 hr at room temperature. The resulting solution was poured into iced ether-saturated aqueous NH₄Cl solution (400 mL, 3:1 v/v). The organic layer was separated and the aqueous layer was washed with ether (150 mL×2). The combined organic layer was dried over MgSO₄ and the solvent was removed under the reduced pressure. The residue was purified by silica gel column chromatography (hexane:EtOAc=10:1 to 3:1 v/v) to give compound 7b (35.64 g, 78.10 mmol) in 100% yield. R_{f}=0.3 (hexane:EtOAc=10:1 v/v); ¹H-NMR (CDCl₃, 500 MHz) δ 7.68 (m, 4H), 7.48-7.38 (m, 6H), 6.06 (m, 1H), 5.49 (dt, J=17.0, 1.5, 1H), 5.27 (dt, J=10.0, 1.5, 1H), 4.36 (m, 1H), 4.25 (d, J=3.0, 1H), 4.12 (dd, J=10.0, 5.5, 1H), 4.05 (dd, J=10.0, 5.5, 1H), 3.92 (m, 2H), 3.77 (m, 1H), 3.42 (d, J=3.0, 1H), 1.31 (s, 3H), 1.26 (s, 3H), 1.09 (s, 9H); ¹³C-NMR (CDCl₃, 125 MHz) δ 135.77, 135.60, 130.43, 130.25, 128.14, 128.08, 127.95, 112.15, 103.41, 87.31, 87.09, 81.74, 65.53, 26.95, 26.91, 26.52, 25.02, 19.18; HRMS (ES) calcd for C₂₆H₃₆O₅Si (M+Na⁺) 455.1835, found 455.1877.

### 1-{5-[1-tert-Butyl-dimethyl-silanyloxy)-allyl]-2,2-dimethyl-[1,3]dioxolan-4-yl}-2-(tert-butyl-diphenyl-silanyloxy)-ethanol (8b)

To a solution of compound 7b (28.0 g, 61.14 mmol) in 200 mL of anhydrous CH₂Cl₂-DMF solution (10:1 v/v), imidazole (12.48 g, 183.45 mmol) and TBDMSCl (10.20 g, 67.50 mmol) were added at 0° C. under nitrogen atmosphere. The reaction mixture was stirred for 24 h at room temperature and then poured into 600 mL of co-solvent (ether-water, 1:1 v/v). The organic layer was separated and the aqueous layer was washed with ether (100 mL×2). The combined organic layer was washed with brine (150 mL×2), dried over MgSO₄, concentrated. The residue was purified by silica gel column chromatography (hexane:EtOAc=20:1 v/v) to give compound 8b (31.42 g, 55.03 mmol) in 90% yield. R_{f}=0.4 (Hex:EtOAc=20:1 v/v); ¹H-NMR (CDCl₃, 500 MHz) δ 7.72 (m, 4H), 7.42-7.33 (m, 6H), 5.90 (m, 1H), 5.25 (dd, J=16.5, 11.5, 2H), 5.27 (dt, J=10.0, 1.5, 1H), 4.28 (t, J=6.0, 1H), 4.15 (dd, J=10.0, 5.0, 1H), 4.06 (t, J=5.0, 1H), 3.92 (m, 2H), 3.80 (dd, J=10.0, 6.0, 1H), 3.58 (d, J=5.0, 1H), 1.29 (s, 3H), 1.28 (s, 3H), 1.05 (s, 9H), 0.92 (s, 9H), 0.14 (s, 3H), 0.11 (s, 3H); ¹³C-NMR (CDCl₃, 125 MHz) δ 143.05, 140.82, 140.75, 138.80, 138.65, 134.59, 134.57, 132.66, 132.60, 123.15, 113.18, 85.07, 81.93, 78.81, 74.76, 70.54, 32.83, 31.90, 31.86, 31.01, 30.74, 24.38, 23.32, 1.32, 0.71; HRMS (ES) calcd for C₃₂H₅₀O₅Si₂ (M+H⁺) 571.3276, found 571.3260.

### 1-{5-[1-(tert-Butyl-dimethyl-silanyloxy)-allyl]-2,2-dimethyl-[1,3]dioxolan-4-yl}-2-(tert-butyl-diphenyl-silanyloxy)ethanone (9a)

To a solution of oxalyl chloride (1.38 g, 10.95 mmol) in 20 mL of anhydrous CH₂Cl₂, DMSO (1.72 g, 21.98 mmol) was added at −60° C. under nitrogen atmosphere and then the resulting solution was stirred for 10 min. A solution of compound 8a (5.0 g, 8.76 mmol) in 50 mL of anhydrous CH₂Cl₂ was added to the reaction mixture dropwise over 15 min at −60° C. After another 30 min, Et₃N (4.43 g, 43.79 mmol) was added dropwise at −60° C. to the reaction mixture. The mixture was allowed to stir for 30 min at −60° C. and then stirring for 30 min, to the reaction mixture was added 100 mL of cold water and the aqueous layer was separated and extracted with CH₂Cl₂ (50 mL×3). The combined organic layer was washed with brine (50 mL×2), dried over MgSO₄, concentrated under reduced pressure and purified by silica gel column chromatography (Hex:EtOAc=20:1 to 10:1 v/v) to give compound 9b (4.74 g, 8.32 mmol) in 95% yield. R_{f}=0.5 (hexane:EtOAc=20:1 v/v); [α]²³_{D} −22.81 (c 1.00, CHCl₃); ¹H-NMR (CDCl₃, 500 MHz) δ 7.62 (m, 4H), 7.40-7.29 (m, 6H), 5.82 (m, 1H), 5.13 (s, 1H), 5.11 (dd, J=17.0, 10.0, 1H), 4.56 (d, J=7.5, 1H), 4.51 (d, J=18.5, 1H), 4.38 (d, J=18.5, 1H), 4.32 (dd, J=7.5, 3.5, 1H), 4.28 (dd, J=7.0, 3.5, 1H), 1.39 (s, 3H), 1.23 (s, 3H), 1.01 (s, 9H), 0.84 (s, 9H), 0.03 (s, 6H); ¹³C-NMR (CDCl₃, 125 MHz) δ 210.06, 142.00, 140.10, 140.03, 137.64, 137.31, 134.26, 134.25, 132.19, 132.16, 122.47, 113.74, 86.66, 83.55, 77.95, 73.54, 31.23, 30.89, 30.52, 29.04, 23.77, 22.87, 0.23, 0.00; HRMS (ES) calcd for C₃₂H₄₈O₅Si₂ (+Na⁺) 591.2938, found 591.2969.

### 4-[1-(tert-Butyl-dimethyl-silanyloxy)-allyl]-5-[1-(tert-butyl-diphenyl-silanyloxymethyl)-vinyl]-2,2-dimethyl-[1,3]dioxolane (10b)

To a suspension of Ph₃PCH₃Br (15.54 g, 43.50 mmol) in 50 mL of THF, 25 mL of n-BuLi (1.6 M in hexane) was added at 0° C. under N₂ atmosphere. After 30 min, a solution of compound 9b (4.50 g, 7.91 mmol) in 100 mL of THF was added to the reaction mixture at 0° C. The resulting solution was allowed to stir for 12 hr at room temperature, then treated with 20 mL of MeOH and 40 mL of water and then poured into ether-water solution (200 mL, 3:1 v/v). The organic layer was separated and the aqueous layer was washed with ether (100 mL×2). The collected solution was washed with brine (50 mL×2), dried over MgSO₄ and purified by silica gel column chromatography (hexane:EtOAc=50:1 to 10:1 v/v) to give compound 10b (4.17 g, 7.36 mmol) in 93% yield. R_{f}=0.6 (hexane:EtOAc=40:1 v/v); ¹H-NMR (CDCl₃, 500 MHz) δ 7.72 (m, 4H), 7.48-7.40 (m, 6H), 5.78 (m, 1H), 5.148 (s, 1H), 5.38 (s, 1H), 5.20 (d, J=10.0, 1H), 5.14 (d, J=17.0, 1H), 4.80 (d, J=6.5, 1H), 4.25 (dd, J=14.0, 17.0, 1H), 4.05 (t, J=7.5, 1H), 3.98 (dd, J=6.5, 1H), 1.39 (s, 3H), 1.35 (s, 3H), 1.12 (s, 9H), 0.85 (s, 9H), 0.02 (s, 3H), 0.00 (s, 3H); ¹³C-NMR (CDCl₃, 125 MHz) δ 147.28, 142.91, 139.67, 139.63, 137.59, 137.53, 133.84, 133.81, 131.86, 131.84, 131.82, 121.39, 116.30, 111.92, 84.68, 81.82, 77.46, 69.08, 30.98, 30.81, 30.23, 29.24, 23.42, 22.31, 0.73, 0.00; HRMS (ES) calcd for C₃₃H₅₀O₅Si₂ (M+Na⁺) 589.3146, found 589.3181.

### 1-[5-(1-Hydroxymethyl-vinyl)-2,2-dimethyl-[1,3]dioxolan-4-yl]-prop-2-en-1-ol (11b)

To a solution of compound 10b (0.84 g, 1.48 mmol) in 10 mL of THF, 4.0 mL of TBAF in THF solution (1.0 M in THF) was added at room temperature. After stirring for 2 hr, the reaction mixture was adsorbed on silica gel and then purified by silica gel column chromatography (hexane:EtOAc=2:1 to 1:2 v/v) to give compound 11b (0.31 g, 1.42 mmol) in 95% yield. R_{f}=0.2 (hexane:EtOAc=5:1 v/v); [α]²⁴_{D} −161.30 (c 0.50, CHCl₃); ¹H-NMR (CDCl₃, 500 MHz) δ 5.97 (m, 1H), 5.40 (s, 1H), 5.27 (d, J=17.0, 1H), 5.25 (s, 1H), 5.19 (d, J=10.0, 1H), 4.77 (d, J=6.0, 1H), 4.46 (br, 1H), 4.16 (s, 1H), 3.98 (m, 2H), 3.20 (m, 1H), 1.44 (s, 3H), 1.34 (s, 3H); ¹³C-NMR (CDCl₃, 125 MHz) δ 143.35, 138.21, 116.27, 113.09, 107.75, 80.28, 77.74, 69.81, 65.44, 27.51, 25.23, 23.94, 19.67, 13.70; HRMS (ES) calcd for C₁₁H₁₈O₄ (M+Na⁺) 237.1103, found 237.1106.

### 6-Hydroxymethyl-2,2-dimethyl-4,6a-dihydro-3aH-cyclopenta[1,3]dioxo-4-ol (14)

Method A: To a solution of compound 11b (0.14 g, 0.65 mmol) of 50 mL of anhydrous CH₂Cl₂, 0.05 equiv of 2 ^{nd}-generation Grubbs catalyst (0.028 g, 0.03 mmol) was added at room temperature under argon atmosphere. After stirring for 24 hr, the reaction mixture was adsorbed on silica gel and then purified by silica gel column chromatography (hexane:EtOAc=1:1 to 1:2 v/v) to give compound 14 (0.11 g, 0.60 mmol) in 92% yield. R_{f}=0.2 (Hex:EtOAc=1:2 v/v); ¹H-NMR (CDCl₃, 500 MHz) δ 5.75 (s, 1H), 4.98 (d, J=5.0, 1H), 4.79 (dd, J=5.0, 6.0, 1H), 4.57 (m, 1H), 4.31 (dd, J=14.5, 33.5, 2H), 2.87 (d, J=10.5, 1H), 2.60 (br, 1H), 1.45 (s, 3H), 1.41 (s, 3H); ¹³C-NMR (CDCl₃, 125 MHz) δ 144.86, 129.95, 112.61, 83.08, 77.89, 73.22, 59.47, 27.55, 26.46; HRMS (ES) calcd for C₉H₁₄O₄ (M+Na⁺) 209.0790, found 209.0821.

Method B: To a solution of compound 11b (0.040 g, 0.187 mmol) in 10 mL of anhydrous CH₂Cl₂, 0.10 equiv of 1st-generation Grubbs catalyst (0.016 g, 0.019 mmol) was added at room temperature under argon atmosphere. After stirring for 24 hr, the reaction mixture was adsorbed on silica gel and then purified by silica gel column chromatography (hexane:EtOAc=1:1 to 1:2 v/v) to give compound 14 (0.032 g, 0.171 mmol) in 90% yield.

### Scheme 2, FIG. 2

### 1-(2,2-Dimethyl-6-trityloxymethyl-4,6a-dihydro-3aH-cyclopenta[1,3]dioxol-4-yl)-1H-[1,2,4]-triazole-3-carboxylic acid methyl ester (15a)

To a solution of Ph₃P (0.60 g, 2.10 mmol) in 2.0 mL of anhydrous THF, 0.35 mL of DIAD was added at 0° C. under N₂ atmosphere, then stirred for 30 min. A solution of compound (+)-12a (0.30 g, 0.70 mmol) in 5.0 mL of anhydrous THF was added to the reaction mixture at −78° C., then stirred for additional 30 min. To the suspension, was added methyl-1H-1,2,4-triazole-3-carboxylate (0.15 g, 1.05 mmol) at −78° C. and then the reaction mixture was allowed to stir for 24 hr at room temperature. The reaction mixture was purified by silica gel column chromatography (hexane:EtOAc=2:1 to 1:2 v/v) to give compound 15a (0.38 g, 0.70 mmol) with a little amount of DIAD. ¹H-NMR (CDCl₃, 500 MHz) δ 7.98 (s, 1H), 7.45 (m, 6H), 7.30-7.21 (m, 9H), 6.45 (s, 1H) 6.03 (t, J=2.0, 1H), 5.27 (d, J=5.5, 1H), 4.84 (t, J=5.5, 1H), 4.05 (s, 3H), 3.98 (dt, J=15.5, 2.0, 1H), 3.76 (d, J=15.5, 1H), 1.42 (s, 3H), 1.32 (s, 3H); ¹³C-NMR (CDCl₃, 125 MHz) δ 161.14, 151.22, 143.65, 140.04, 128.55, 128.02, 127.31, 126.39, 121.19, 112.94, 87.39, 84.43, 83.79, 70.42, 61.33, 52.33, 27.45, 26.08; HRMS (ES) calcd for C₃₂H₃₁N₃O₅ (M+H⁺) 538.2342, found 538.2370.

### 1-(2,2-Dimethyl-6-trityloxymethyl-4,6a-dihydro-3aH-cyclopenta[1,3]dioxol-4-yl)-1H-[1,2,4]-triazole-3-carboxylic acid amide (16a)

The crude compound 15a (0.38 g, 0.70 mmol) was dissolved in 20.0 mL of saturated methanolic ammonia at 0° C. and then the solution was allowed to stir for 12 hr at room temperature. The solvent and ammonia were evaporated under reduced pressure and the residue was purified by silica gel column chromatography (hexane:EtOAc=1:1 v/v) to give compound 16a (0.34 g, 0.65 mmol) in 92% yield from (+)-12a. ¹H-NMR (CDCl₃, 500 MHz) δ 7.87 (s, 1H), 7.44 (m, 6H), 7.30-7.21 (m, 9H), 6.69 (s, 1H), 6.06 (d, J=2.0, 1H), 5.87 (br, 2H), 5.27 (d, J=10.0, 1H), 4.87 (d, J=10.0 1H), 3.96 (dt, J=15.5, 2.0, 1H), 3.73 (d, J=15.5, 1H), 1.42 (s, 3H), 1.32 (s, 3H); ¹³C-NMR (CDCl₃, 125 MHz) δ 158.49, 151.16, 149.20, 144.14, 143.79, 128.59, 127.91, 127.15, 122.88, 112.50, 87.18, 84.57, 84.13, 71.89, 69.66, 61.42, 53.34, 27.62, 26.35; (EMS (ES) calcd for C₃₁H₃₀N₄O₄ (M+Na⁺) 545.2165, found 545.2125; Anal. Calcd for C₃₁H₃₀N₄O₄.0.1H₂O: C, 71.00; H, 5.80; N, 10.68. Found: C, 70.87; H, 5.80; N, 10.73.

### 1-(4,5-Dihydroxy-3-hydroxymethyl-cyclopenten-2-enyl)-1H-[1,2,4]triazole-3-carboxylic acid amide (17a)

The solution of compound 16a (0.20 g, 0.38 mmol) in 5.0 mL of MeOH was treated with 20 mL of ethereal HCl (1.0 M in ether) solution at 0° C. The acidic solution was allowed to stir for 2 hr at room temperature. The solvents and hydrogen chloride was evaporated under vacuum and the residue was purified by reverse silica gel column chromatography (distilled water) to give compound 17a (0.08 g, 0.35 mmol) in 88% yield. UV (H₂O)λₘₐₓ 225.0 (ε 10203, pH 11), 204.0 (ε 8587, pH 7), 199 (ε 9959, pH 2); [α]²⁶_{D} −146.93 (c 1.00, CH₃OH); ¹H-NMR (CD₃OD, 500 MHz) δ 8.13 (s, 1H), 6.44 (d, J=2.5, 1H), 5.81 (dd, J=8.5, 2.5, 1H), 4.64 (d, J=5.5, 1H), 4.46 (dd, J=8.5, 5.5, 1H), 4.30 (m, 2H); ¹³C-NMR (CD₃OD, 125 MHz) δ 158.93, 148.98, 148.81, 146.85, 124.70, 77.27, 72.87, 69.69, 58.89; HRMS (ES) calcd for C₉H₁₂N₄O₄(M+Na⁺) 263.0757, found 263.0792; Anal. Calcd for C₉H₁₂N₄O₄.0.25H₂O: C, 44.17; H, 5.15; N, 22.89. Found: C, 44.53; H, 5.15; N, 22.50.

### 1-2,2-Dimethyl-6-trityloxymethyl-4,6a-dihydro-3aH-cyclopenta[1,3]dioxol-4-yl)-1H-imidazole-4-carboxylic acid methyl ester (15b)

Compound 15b (0.39 g, 0.70 mmol) was synthesized from (+)-12a (0.30 g, 0.70 mmol) and methyl-4-imidazolecarboxylate (0.15 g, 1.05 mmol) following the same procedure as the compound 15a. ¹H-NMR (CDCl₃, 500 M ) δ 7.81 (s, 1H), 7.47 (m, 6M), 7.33-7.24 (m, 9H), 6.05 (s, 1H) 5.23 (s, 1H), 5.11 (d, J=5.0, 1H), 4.53 (t, J=5.0, 1H), 4.03 (d, J=15.0 1H), 3.90 (s, 3H), 3.83 (d, J=15.0, 1H), 1.41 (s, 3H), 1.32 (s, 3H); ¹³C-NMR (CDCl₃, 125 MHz) δ 160.75, 153.57, 150.51, 143.72, 138.61, 128.55, 127.98, 127.25, 122.02, 112.49, 87.26, 85.33, 83.66, 71.82, 66.29, 61.23, 51.78, 27.60, 26.33; HRMS (ES) calcd for C₃₃H₃₂N₂O₅ (M+H⁺) 537.2389, found 537.2396.

### 1-(2,2-Dimethyl-6-trityloxymethyl-4,6a-dihydro-3aH-cyclopenta[1,3]dioxol-4-yl)-1H-imidazole-4-carboxylic acid amide (16b)

The crude compound 15b (0.39 g, 0.70 mmol) was dissolved in 10.0 mL of saturated methanolic ammonia at 0° C. and then the solution was allowed to stir for 24 hr at 100° C. The solvent and ammonia were evaporated under reduced pressure and the residue was purified by silica gel column chromatography (hexane:EtOAc=1:1 to EtOAc v/v) to give compound 16b (0.33 g, 0.63 mmol) in 90% yield from (+)-12a. ¹H-NMR (CDCl₃, 500 M ) δ 7.87 (s, 1H), 7.44 (m, 6H), 7.30-7.21 (m, 9H), 6.69 (s, 1H), 6.06 (d, J=2.0, 1H), 5.87 (br, 2H), 5.27 (d, J=10.0, 1H), 4.87 (d, J=10.0 1H), 3.96 (dt, J=15.5, 2.0, 1H), 3.73 (d, J=15.5, 1H), 1.42 (s, 3H), 1.32 (s, 3H); ¹³C-NMR (CDCl₃, 125 MHz) δ 158.81, 150.11, 148.74, 145.55, 143.83, 128.60, 127.90, 127.12, 123.35, 112.35, 87.14, 84.65, 84.07, 69.38, 61.45, 27.64, 26.37; HRMS (ES) calcd for C₃₂H₃₁N₃O₄ (M+H⁺) 522.2393, found 537.2358.

### 1-(4,5-Dihydroxy-3-hydroxymethyl-cyclopenten-2-enyl)-1H-imidazole-4-carboxylic acid amide (17b)

Compound 17b (0.10 g, 0.45 mmol) was prepared in 85% yield from 16b (0.25 g, 0.48 mmol) following the same procedure as the compound 17a. UV (H₂O) λₘₐₓ 237.0 (ε 2455, pH 11), 204.0, 239 (ε 5132, 4034, pH 7), 198 (ε 7180, pH 2); [α]²⁵_{D} −15.43 (c 0.50, CH₃OH); ¹H-NMR (D₂O, 500 MHz) δ 7.88 (br, 1H), 7.61 (br, J=2.5, 1H), 5.83 (d, J=1.5, 1H), 5.79 (s, 1H), 4.53 (d, J=5.5, 1H), 4.21 (s, 2H), 4.06 (t, J=5.5, 1H); ¹³C-NMR (D₂O, 125 MHz) δ 163.99, 148.76, 131.90, 124.93, 78.71, 72.81, 65.60, 58.56, 48.80; HRMS (ES) calcd for C₁₀H₁₃N₃O₄ (M+Na⁺) 262.0804, found 262.0826; Anal. Calcd for C₁₀H₁₄N₃O₄.1.0HCl.0.30H₂O: C, 43.28; H, 5.16; N, 15.14. Found: C, 43.66; H, 5.20; N, 15.25.

### 4-Azido-2,2-dimethyl-6-tritloxymethyl-4,6a-dihydro-3aH-cyclopenta[1,3]dioxole (18)

To a solution of (+)-12a (0.35 g, 0.82 mmol) in 20 mL of anhydrous CH₂Cl₂, MsCl (0.15 mL, 1.64 mmol) and Et₃N (0.40 mL, 2.86 mmol) were added at 0° C. under N₂ atmosphere. After stirring the mixture for 1 hr at 0° C., the reaction mixture was poured into 50 mL of water-CH₂Cl₂ (1:5 v/v) solution. The organic layer was separated and washed with brine (10 mL×2) and dried over MgSO₄. The solution was concentrated and the residue was purified on a short silica gel pad. To the crude product (0.42 g, 0.82 mmol), were directly added 15 mL of anhydrous DMF and NaN₃ (0.44 g, 6.70 mmol) at room temperature under N₂ atmosphere, then stirred for 24 hr at 80° C. The reaction mixture was poured into 100 mL of iced ether-water (5:1 v/v) and then the organic layer was separated and the aqueous layer was washed with ether (25 mL×2). The collected organic layer was washed with brine (20 mL×2), dried over MgSO₄, filtered and concentrated under reduced pressure. The residue was purified by silica gel column chromatography (hexane:EtOAc=10:1 v/v) to give compound 18 (0.34 g, 0.76 mmol) in 92% yield. ¹H-NMR (CDCl₃, 500 MHz) δ 7.46 (m, 6H), 7.44-7.22 (m, 9H), 6.01 (d, J=1.5, 1H), 5.04 (d, J=5.5, 1H), 4.58 (d, J=5.5, 1H), 4.42 (s, 1H), 3.87 (td, J=1.5, J=15.0, 1H), 3.74 (d, J=15.0, 1H), 1.33 (s, 3H), 1.30 (s, 3H); ¹³C-NMR (CDCl₃, 125 MHz) δ 149.01, 143.84, 128.60, 127.98, 127.21, 122.70, 112.14, 87.21, 84.06, 83.83, 69.99, 61.29, 27.49, 26.22; IR (neat) 3072, 2988, 2097, 1265, 1082, 735, 701 cm⁻¹.

### 1-(2,2-Dimethyl-6-trityloxymethyl-4,6a-dihydro-3aH-cyclopenta[1,3]dioxol-4-yl)-1H-[1,2,3]-triazole-4-carboxylic acid methyl ester (15c)

To a solution of compound 18 (0.18 g, 0.40 mmol) in 10 mL of THF, CuI (0.76 g, 4.0 mmol), methyl propiolate (0.14 g, 1.60 mmol) and Et₃N (1.22 g, 12.0 mmol) were added at room temperature under N₂ atmosphere, and then stirred for 12 hr. The suspension was adsorbed on silica gel and then purified by silica gel column chromatography (hexane:EtOAc=10:1 to 1:2 v/v) to give compound 15c (0.21 g, 0.39 mmol) in 98% yield. R_{f}=0.2 (hexane:EtOAc=5:1 v/v); ¹H-NMR (CDCl₃, 500 MHz) δ 7.99 (s, 1H), 7.46 (m, 6H), 7.33-7.22 (m, 9H), 6.06 (s, 1H) 5.74 (s, 1H), 5.17 (d, J=6.0, 1H), 4.71 (d, J=6.0, 1H), 4.03 (d, J=16.0 1H), 3.98 (s, 3H), 3.83 (d, J=16.0, 1H), 1.42 (s, 3H), 1.31 (s, 3H); ¹³C-NMR (CDCl₃, 125 MHz) δ 161.14, 151.22, 143.65, 140.04, 128.55, 128.02, 127.31, 126.39, 121.19, 112.94, 87.39, 84.43, 83.79, 70.42, 61.33, 52.33, 27.45, 26.08; HRMS (ES) calcd for C₃₂H₃₁N₃O₅ (M+H⁺) 560.2162, found 560.2114.

### 1-(2,2-Dimethyl-6-trityloxymethyl-4,6a-dihydro-3aH-cyclopenta[1,3]dioxol-4-yl)-1H-[1,2,3]-triazole-4-carboxylic acid amide (16c)

The solution of compound 15c (0.19 g, 0.36 mmol) in 40 mL of MeOH, NH₃ gas was bubbled with at 0° C. for 30 min. and then the solution was allowed to stir for 24 hr at room temperature. The solvent and NH₃ were removed in vacuo. The solvent was removed under high vacuum for 24 hr at room temperature to give the product 16c in 100% (0.19 g, 0.36 mmol). R_{f}=0.2 (hexane:EtOAc=2:1 v/v); ¹H-NMR (CDCl₃, 500 MHz) δ 8.06 (s, 1H), 7.45 (m, 6H), 7.32-7.21 (m, 9H), 7.19 (d, J=2.0, 1H), 6.21 (d, J=2.0, 1H), 6.07 (d, J=1.5, 1H), 5.74 (s, 1H), 5.18 (d, J=5.5, 1H), 4.71 (d, J=5.5, 1H), 4.16 (dt, J=16.0, 1.5, 1H), 3.81 (d, J=16.0, 1H), 1.41 (s, 3H), 1.31 (s, 3H); ¹³C-NMR (CDCl₃, 125 MHz) δ 162.15, 151.12, 143.65, 142.83, 128.55, 128.02, 127.30, 124.78, 121.27, 112.89, 87.33, 84.43, 83.84, 70.37, 61.28, 27.46, 26.13; HRMS (ES) calcd for C₃₁H₃₀N₄O₄ (M+H⁺) 523.2346, found 523.2370.

### 1-(4,5-Dihydroxy-3-hydroxymethyl-cyclopenten-2-enyl)-1H-[1,2,3]triazole-4-carboxylic acid amide (17c)

Compound 17c was prepared in 88% yield (0.08 g, 0.33 mmol) from 16c (0.20 g, 0.38 mmol) following the same procedure as the compound 17a. R_{f}=0.1 (CH₂Cl₂: MeOH=10:1 v/v); mp 178-180 0° C.; UV (H₂O) λₘₐₓ 225.0 (ε 7014, pH 11), 204.0 (ε 6463, pH 7), 199 (ε 10503, pH 2); [α]²⁵_{D} −127.56 (c=1.0, MeOH); ¹H-NMR (CD₃OD, 500 MHz) δ 8.46 (s, 1H), 5.95 (s, 1H), 5.61 (s, 1H), 5.52 (s, 1H), 4.63 (d, J=5.5, 1H), 4.30 (m, 3H); ¹³C-NMR (CD₃OD, 125 MHz) δ 150.63, 125.35, 123.39, 78.24, 73.02, 58.95, 53.86, 48.68; Anal. Calcd for C₉H₁₂N₄O₄.0.3H₂O: C, 44.01; H, 5.17; N, 22.81. Found: C, 44.19; H, 5.06; N, 22.79.

### Experimental/Analytical Results for 3-Deazaadenine Synthesis and (−)-3-Deazaneplanocin A

N9-isomer (16d): UV (H₂O) λₘₐₓ 269.0 (ε 2455, pH 11), 267.0 (ε 5134 pH 7.4); [α]²⁷_{D} −11.64 (c 1.00, CH₃Cl); NMR (CDCl₃, 500 MHz) δ 8.36 (d, J=6.0, 1H), 7.87 (s, 1H), 7.47 (d, J=7.5, 6H), 7.42 (d, J=6.0, 1H), 7.34-7.25 (m, 9H), 6.15 (s, 1H), 5.45 (s, 1H), 5.19 (d, J=5.5, 1H), 4.59 (d, J=5.5, 1H), 4.04 (d, J=15.0, 1H), 4.90 (d, J=15.0, 1H), 1.46 (s, 3H), 1.45 (s, 18H), 1.32 (s, 3H); ¹³C-NMR (CDCl₃, 125 MHz) δ 151.44, 150.47, 144.75, 143.64, 142.34, 141.14, 140.18, 137.30, 128.55, 128.05, 121.58, 112.98, 106.12, 87.41, 84.56, 83.85, 82.98, 66.66, 61.32, 27.98, 27.36, 25.85; IR (neat, cm⁻¹) 3060, 2973, 2926, 1783, 1748, 1608, 1485, 1450; HRMS (ES) calcd for C₁₂H₁₄N₄O₃ (M+H⁺) 745.3597, found 745.3553.

N7-isomer (17d): UV (H₂O) λₘₐₓ 278.0 (ε 2455, pH 11), 277.0 (ε 5134 pH 7.4); [α]²⁷_{D} +25.77 (c 0.98, CH₃Cl); NMR (CDCl₃, 400 MHz) δ 8.36 (d, J=5.6, 1H), 7.83 (s, 1H), 7.72 (d, J=5.6, 1H), 7.48 (d, J=7.2, 6H), 7.35-7.25 (m, 9H), 6.07 (s, 1H), 5.55 (s, 1H), 5.16 (d, J=6.0 1H), 4.56 (d, J=6.0, 1H), 4.09 (d, J=16.0, 1H), 3.90 (d, J=16.0, 1H), 1.46 (s, 9H), 1.42 (s, 3H), 1.39 (s, 9H), 1.29 (s, 3H); ¹³C-NMR (CDCl₃, 100 MHz) δ 151.96, 151.55, 150.99, 150.54, 143.74, 143.63, 140.69, 137.48, 128.51, 127.99, 127.47, 127.32, 121.81, 115.64, 112.83, 87.40, 85.09, 83.81, 83.41, 83.27, 66.54, 61.33, 28.02, 27.94, 27.68, 26.51; IR (neat, cm⁻¹) 3055, 2980, 2926, 1784, 1750, 1608, 1488, 1449.

### Examples for 7-Deaza-7-Substituted Neplanocin A

### Following Schemes 1-5 for 7-Deaza-7-Substituted-Neplanocin A Analogs

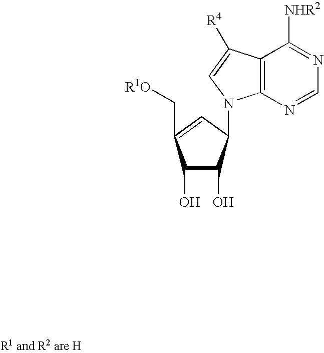

### Experiment Section

Melting points were determined on a Mel-temp II apparatus and are uncorrected. Nuclear magnetic resonance spectra were recorded on a Varian Inova 500 spectrometer at 500 MHz for ¹H NMR and 125 MHz for ¹³C NMR with tetramethylsilane as the internal standard. Chemical shifts (δ) are reported as s (singlet), d (doublet), t (triplet), q (quartet), m (multiplet), or bs (broad singlet). UV spectra were recorded on a Beckman DU-650 spectrophotometer. Optical rotations were measured on a Jasco DIP-370 digital polarimeter. TLC was performed on Uniplates (silica gel) purchased from Analtech Co. Column chromatography was performed using either silica gel-60 (220-440 mesh) for flash chromatography or silica gel G (TLC grade, >440 mesh) for vacuum flash column chromatography. Elemental analyses were performed by Atlantic Microlab Inc., Norcross, Ga.

### Following Scheme 1

### Compound 6 (7-F-Isopropylidine Analog of Scheme 1 7-Deaza-7-Substituted Neplanocin A)

To a mixture of compound 1 (318 mg, 0.74 mmol), compound 2 (140 mg, 0.82 mmol) and Ph₃P (388 mg, 1.48 mmol) in THF (30 mL) was added DIAD (299 mg, 1.48 mmol) at 0° C. After stirring 30 min at rt, the mixture was evaporated and purified by column chromatography on a silica gel. The appropriate fraction was collected and evaporated. The residue was treated with saturated methanolic ammonia and heated to 80° C. for 16 h. After cooling, the mixture was purified by column chromatography on a silica gel to give 6 (350 mg, 84%) as a white solid. mp: 100-102° C.; [α]²⁷_{D} −25.33 (c 0.13, CHCl₃); ¹H NMR (500 MHz, CDCl₃) δ 8.33 (s, 1H), 7.4-7.5 (m, 6H), 7.2-7.35 (m, 9H), 6.48 (d, J=2.0 Hz, 1H), 5.99 (s, 1H), 5.88 (s, 1H), 5.37 (s, 2H), 5.18 (d, J=6.0 Hz, 1H), 4.54 (d, J=6.0 Hz, 1H), 4.01 (dt, J=15 and 2 Hz, 1H), 3.83 (d, J=15.5 Hz, 1H), 1.42 (s, 3H), 1.30 (s, 3H); ¹³C NMR (100 MHz, CDCl₃) 155.51, 155.49, 153.20, 149.12, 145.84, 145.81, 144.14, 143.78, 141.71, 128.54, 128.09, 127.94, 127.20, 123.07, 112.48, 104.25, 103.99, 93.81, 93.66, 87.26, 84.99, 83.94, 77.24, 63.97, 61.48, 27.52, 26.10. UV (CHCl₃) λₘₐₓ 283.0 nm; Anal. Calcd. for (C₃₄H₃₁FN₄O₃.0.34CHCl₃) C, 68.37; H, 5.24; N, 9.29. Found C, 68.40; H, 5.17; N, 9.08.

### Compound 10 (7-F-Substituted Compound)

A mixture of compound 6 (300 mg) and HCl (0.2 mL) in MeOH (10 mL), THF (10 mL) and water (1 mL) was heated to 50° C. for 16 h. It was neutralized by NaHCO₃ and purified by column chromatography on silica gel to give 10 (140 mg, 94%) as a white solid. mp: 224-225° C.; [α]²⁶_{D} −102.29 (c 0.14, MeOH); ¹HNMR (500 MHz, DMSO-d₆) δ 8.08 (s, 1H), 7.01 (d, J=2.5 Hz, 1H), 6.94 (br s, 2H), 5.59 (br s, 2H), 5.02 (d, J=6.5 Hz, 1H), 4.92 (d, J=6.5 Hz, 1H), 4.90 (t, J=5.5 Hz, 1H), 4.42 (t, J=6.0 Hz, 1H), 4.12 (m, 2H), 4.08 (m, 1H); ¹³C NMR (100 M , DMSO-d₆) 156.22, 156.20, 152.96, 150.52, 146.15, 146.12, 143.86, 141.44, 124.47, 104.73, 104.47, 92.57, 92.42, 77.34, 72.70, 63.71, 58.99. UV (H₂O)λₘₐₓ 282 nm (pH 2), 282 nm (pH 7), 283 nm (pH 11); Anal. Calcd. for (C₁₂H₁₃FN₄O₃.0.2H₂O) C, 50.78; H, 4.76; N, 19.74. Found C, 50.77; H, 4.78; N, 19.50.

### Compound 7 (7-Cl-Substituted Isopropylidine Analog)

To a mixture of compound 1 (193 mg, 0.45 mmol), compound 3 (110 mg, 0.59 mmol) and Ph₃P (177 mg, 0.68 mmol) in THF (20 mL) was added DIAD (0.13 mL, 0.68 mmol) at 0° C. After stirring 30 min at rt, the mixture was evaporated and purified by column chromatography on a silica gel. The appropriate fraction was collected and evaporated. The residue was treated with saturated methanolic ammonia and heated to 80° C. for 16 h. After cooling, the mixture was purified by column chromatography on a silica gel to give 7 (205 mg, 79%) as a white solid. mp: 94-95° C.; [α]²⁸_{D} −28.55 (c 0.20, CHCl₃); ¹H NMR (500 MHz, CDCl₃) δ 8.34 (s, 1H), 7.4-7.5 (m, 6H), 7.2-7.35 (m, 9H), 6.68 (s, 1H), 5.99 (s, 1H, 5.85 (s, 1H), 5.57 (br s, 2H), 5.17 (d, J=5.5 Hz, 1H), 4.54 (d, J=5.5 Hz, 1H), 4.03 (m, 1H), 3.84 (d, J=16.0 Hz, 1H), 1.43 (s, 3H), 1.30 (s, 3H); ¹³C NMR (125 MHz, CDCl₃) 156.62, 153.20, 149.51, 149.02, 143.81, 128.58, 127.98, 127.25, 122.80, 118.67, 112.56, 103.10, 101.34, 87.35, 84.99, 83.98, 64.39, 61.54, 27.55, 26.13. UV (CHCl₃) λₘₐₓ 284.0 nm; Anal. Calcd. for (C₃₄H₃₁ClN₄O₃.0.3CH₃CO₂C₂H₅) C, 69.82; H, 5.56; N, 9.25. Found C, 69.60, H, 5.37, N9.21.

### Compound 11 (7-Deaza-7-Chloro Neplanocin A)

A mixture of compound 7 (190 mg) and HCl (0.2 mL) in MeOH (10 mL), THF (10 mL) and water (1 mL) was heated to 50° C. for 16 h. It was neutralized by NaHCO₃ and purified by column chromatography on silica gel to give 11 (87 mg, 90%) as a white solid. mp: 236-237° C.; [α]²⁷_{D} −140.51 (c 0.14, MeOH); ¹H NMR (400 MHz, DMSO-d₆) δ 8.07 (s, 1H), 7.21 (s, 1H), 6.90 (br s, 2H), 5.56 (d, J=1.2 Hz, 1H), 5.52 (br s, 1H), 5.01 (d, J=7.2 Hz, 1H), 4.89 (d, J=6.0 Hz, 1H), 4.85 (t, J=5.6 Hz, 1H), 4.39 (t, J=5.6 Hz, 1H), 4.1-4.15 (m, 3H); ¹³C NMR (100 MHz, DMSO-d₆) 157.16, 152.87, 150.74, 149.33, 124.29, 119.56, 102.04, 100.19, 77.39, 72.74, 64.32, 59.00. UV (H₂O) λₘₐₓ 285 nm (pH 2), 285 nm (pH 7), 283 nm (pH 11); Anal. Calcd. for (C₁₂H₁₃ClN₄O₃.0.2CH₃OH) C, 48.34; H, 4.59; N, 18.48. Found C, 48.20, H, 4.53, N, 18.45.

### Compound 8 (7-Br-Substituted Isopropylidine Analog)

To a mixture of compound 1 (144 mg, 0.34 mmol), compound 4 (102 mg, 0.44 mmol) and Ph₃P (134 mg, 0.51 mmol) in THF (20 mL) was added DIAD (0.10 mL, 0.51 mmol) at 0° C. After stirring 30 min at rt, the mixture was evaporated and purified by column chromatography on a silica gel. The appropriate fraction was collected and evaporated. The residue was treated with saturated methanolic ammonia and heated to 80° C. for 16 h. After cooling, the mixture was purified by column chromatography on a silica gel to give 8 (197 mg, 93%) as a white solid. mp: 112-114° C.; [α]²⁶_{D} −39.47 (c 0.15, MeOH); ¹H NMR (500 MHz, CDCl₃) δ 8.34 (s, 1H), 7.4-7.5 (m, 6H), 7.2-7.35 (m, 9H), 6.75 (s, 1H), 5.99 (s, 1H), 5.85 (s, 1H), 5.61 (br s, 2H), 5.17 (d, J=5.5 Hz, 1H), 4.54 (d, J=6.0 Hz, 1H), 4.03 (d, J=15.0 Hz, 1H), 3.85 (d, J=15.5 Hz, 1H), 1.43 (s, 3H), 1.30 (s, 3H); ¹³C NMR (125 MHz, CDCl₃) 156.75, 153.01, 149.57, 149.51, 143.81, 128.58, 127.98, 127.26, 122.75, 121.14, 112.56, 103.10, 87.36, 86.71, 84.99, 83.97, 64.51, 61.55, 27.55, 26.13. UV (MeOH)λₘₐₓ 284.0 nm; Anal. Calcd. for (C₃₄H₃₁BrN₄O₃) C, 65.49; H, 5.01; N, 8.99. Found C, 65.54; H, 4.91; N, 8.72.

### Compound 12 (7-Deaza-7-Bromo Neplanocin A)

A mixture of compound 8 (100 mg) and HCl (0.1 mL) in MeOH (5 mL), THF (5 mL) and water (0.5 mL) was heated to 50° C. for 16 h. It was neutralized by NaHCO₃ and purified by column chromatography on silica gel to give 12 (49 mg, 90%) as a white solid. mp: 242-243° C.; [α]²⁶_{D} −142.06 (c 0.21, MeOH); ¹H NMR (400 MHz, DMSO-d₆) δ 8.08 (s, 1H), 7.26 (s, 1H), 6.70 (br s, 2H), 5.56 (br s, 1H), 5.53 (br s, 1H), 5.02 (d, J=6.4 Hz, 1H), 4.90 (d, J=6.4 Hz, 1H), 4.86 (t, J=5.6 Hz, 1H), 4.39 (t, J=6.4 Hz, 1H), 4.0-4.15 (m, 3H); ¹³C NMR (100 MHz, DMSO-d₆) 157.34, 152.68, 150.79, 149.81, 124.28, 122.10, 101.38, 86.02, 77.42, 72.75, 64.47, 59.00. UV (H₂O) λₘₐₓ 285 nm (pH 2), 283 nm (pH 7), 284 nm (pH 11); Anal. Calcd. for (C₁₂H₁₃BrN₄O₃.0.6CH₃OH) C, 41.99; H, 4.31; N, 15.55. Found C, 41.52, H, 4.15, N, 15.63.

### Compound 9 (7-Iodo-Substituted Isopropylidine Analog)

To a mixture of compound 1 (380 mg, 0.89 mmol), compound 5 (310 mg, 1.11 mmol) and Ph₃P (467 mg, 1.78 mmol) in THF (40 mL), was added DIAD (360 mg, 1.78 mmol) at 0° C. After stirring 30 min at rt, the mixture was evaporated and purified by column chromatography on a silica gel to give a white solid (580 mg). A portion of solid (550 mg) was treated with saturated methanolic ammonia and heated to 80° C. for 16 h. After cooling, the mixture was purified by column chromatography on a silica gel to give 9 (545 mg, 94%) as a white solid. mp: 114-116° C.; [α]²⁷_{D} −34.68 (c 0.34, CHCl₃); ¹H NMR (500 MHz, CDCl₃) δ 8.34 (s, 1H), 7.4-7.5 (m, 6H), 7.2-7.35 (m, 9H), 6.84 (s, 1H), 6.00 (s, 1H), 5.85 (s, 1H), 5.71 (br s, 2H), 5.18 (d, J=5.5 Hz, 1H), 4.54 (d, J=6.0 Hz, 1H), 4.03 (d, J=15.0 Hz, 1H), 3.85 (d, J=16.0 Hz, 1H), 1.44 (s, 3H), 1.30 (s, 3H); ¹³C NMR (125 MHz, CDCl₃) 156.93, 152.63, 150.16, 149.56, 143.82, 128.59, 127.98, 128.00, 127.26, 126.41, 122.77, 112.55, 104.59, 87.31, 85.03, 83.98, 64.64, 61.58, 49.36, 27.56, 26.14. UV (CHCl₃) λₘₐₓ 287.0 nm; Anal. Calcd. for (C₃₄H₃₁IN₄O₃.0.27CHCl₃) C, 58.57; H, 4.48; N, 7.97. Found C, 58.52, H, 4.56, N, 7.98.

### Compound 13 (7-Deaza-7-Iodo Neplanocin A)

A mixture of compound 9 (130 mg) and HCl (0.2 mL) in MeOH (10 mL), THF (10 mL) and water (1 mL) was heated to 50° C. for 16 h. It was neutralized by NaHCO₃ and purified by column chromatography on silica gel to give 13 (65 mg, 87%) as a white solid. mp: 230-231° C.; [α]²⁶_{D} −98.93 (c 0.12, MeOH); ¹H NMR (400 MHz, DMSO-d₆) δ 8.08 (s, 1H), 7.28 (s, 1H), 6.60 (br s, 2H), 5.56 (d, J=1.2 Hz, 1H), 5.51 (br s, 1H), 5.00 (d, J=6.8 Hz, 1H), 4.89 (d, J=6.0 Hz, 1H), 4.85 (t, J=5.6 Hz, 1H), 4.39 (t, J=5.6 Hz, 1H), 4.0-4.15 (m, 3H); ¹³C NMR (100 MHz, DMSO-d₆) 157.59, 152.19, 150.74, 150.42, 127.34, 124.37, 103.60, 77.48, 72.75, 64.56, 59.00, 50.91. UV (H₂O) λₘₐₓ 290 nm (pH 2), 286 nm (pH 7), 285 nm (pH 11); Anal. Calcd. for (C₁₂H₁₃IN₄O₃.0.66CH₃OH) C, 37.15; H, 3.88; N, 13.65. Found C, 37.06, H, 3.69, N, 13.46.

### Scheme 2 for 7-Deaza-7-Substituted-Neplanocin A Analogs

### Compound 14

To a mixture of compound 1 (380 mg, 0.89 mmol), compound 5 (310 mg, 1.11 mmol) and Ph₃P (467 mg, 1.78 mmol) in THF (40 mL) was added DIAD (360 mg, 1.78 mmol) at 0° C. After stirring 30 min at rt, the mixture was evaporated and purified by column chromatography on a silica gel to give 14 (580 mg, 93%) as a white solid. mp: 94-95° C.; [α]²⁴_{D} −33.50 (c 0.26, CHCl₃); ¹H NMR (500 MHz, CDCl₃) δ 8.68 (s, 1H), 7.45-7.50 (m, 6H), 7.25-7.35 (m, 9H), 7.17 (s, 1H), 5.99 (d, J=2.0 Hz, 1H), 5.90 (s, 1H), 5.20 (d, J=5.0 Hz, 1H), 4.55 (d, J=6.0 Hz, 1H), 4.07 (d, J=15.5 Hz, 1H), 3.88 (d, J=15.5 Hz, 1H), 1.45 (s, 3H), 1.31 (s, 3H); ¹³C NMR (125 MHz, CDCl₃) 152.80, 151.14, 150.52, 150.49, 143.73, 132.17, 128.56, 128.02, 127.32, 121.92, 117.49, 112.79, 87.44, 84.80, 83.95, 65.33, 61.52, 51.29, 27.51, 26.07. UV (CHCl₃) λₘₐₓ 310, 267, 224 nm.

### Compound 16 (7-Cyano-Substituted Isopropylidine Analog)

A mixture of Bu₃SnCN (183 mg, 0.58 mmol) and Pd(PPh₃)₄ (67 mg, 0.058 mmol) in dichloroethane (5 mL) was heated to reflux for 30 min. To this mixture was added compound 14 (200 mg, 0.29 mmol) in dichloromethane (5 mL) and refluxed overnight. The mixture was evaporated and purified by chromatography on a silica gel to give a crude 15 (˜120 mg) as a white solid. A mixture of crude 15 in saturated methanolic ammonia was heated to 80° C. for 16 h. After cooling, the mixture was purified by column chromatography on a silica gel to give 16 (93 mg, 80%) as a white solid. mp: 242-244° C.; [α]²⁷_{D} −39.80 (c 0.18, CHCl₃); ¹H NMR (400 MHz, CDCl₃) δ 8.43 (s, 1H), 7.45-7.50 (m, 6H), 7.2-7.35 (m, 10H), 5.97 (s, 1H), 5.83 (s, 1H), 5.54 (br s, 2H), 5.17 (d, J=5.6 Hz, 1H), 4.55 (d, J=5.6 Hz, 1H), 4.09 (d, J=15.6 Hz, 1H), 3.89 (dt, J=16.4 Hz, 1H), 1.44 (s, 3H), 1.31 (s, 3H); ¹³C NMR (100 MHz, CDCl₃) 156.53, 154.30, 150.99, 150.23, 143.69, 129.90, 128.53, 127.98, 127.29, 121.48, 115.48, 112.81, 102.84, 87.40, 84.66, 83.87, 83.21, 65.34, 61.43, 27.48, 26.07. UV (CHCl₃) λₘₐₓ 280 nm; Anal. Calcd. for (C₃₅H₃₁N₅O₃) C, 73.79; H, 5.49; N, 12.29. Found C, 73.37; H, 5.63; N, 12.05.

### Compound 17 (7-Deaza-7-Cyano-Neplanocin A)

A mixture of compound 16 (140 mg) and HCl (1.0 mL) in MeOH (20 mL), THF (20 mL) and water (3 mL) was heated to 50° C. for 16 h. It was neutralized by NaHCO₃ and purified by column chromatography on silica gel to give 17 (52 mg, 73%) as a white solid. mp: 178-180° C.; [α]²⁷_{D} −161.12 (c 0.13, MeOH); ¹H NMR (500 MHz, CD₃OD) δ 8.26 (s, 1H), 7.95 (s, 1H), 5.86 (m, 1H), 5.73 (m, 1H), 4.64 (d, J=5.5 Hz, 1H), 4.3-4.4 (m, 2H), 4.26 (t, J=5.5 Hz, 1H); ¹³C NMR (125 MHz, DMSO-d₆) 157.45, 153.84, 151.45, 150.71, 132.96, 123.62, 116.01, 101.84, 82.41, 77.43, 72.78, 65.32, 59.03. UV (H₂O) λₘₐₓ 277 nm (pH 2), 278 nm (pH 7), 278 nm (pH 11); Anal. Calcd. for (C₁₃H₁₃N₅O₃.0.9CH₃OH) C, 52.81; H, 5.29; N, 22.15. Found C, 52.60; H, 5.17; N, 22.36.

### Compound 18 (7-Deaza-7-Amido-Neplanocin A)

A mixture of compound 17 (50 mg) and 30% H₂O₂ (0.4 mL) in NH₄OH (4 mL) was stirred at rt for 1 h. It was evaporated and the residue was dispersed in MeOH/EtOH (2 mL/4 mL) and filtered to give 18 (47 mg, 92%) as a light yellow solid. mp: 260-261° C.; [α]²⁷_{D} −134.41 (c 0.20, H₂O); ¹H NMR (400 MHz, CD₃OD) δ 8.10 (s, 1H), 7.87 (s, 1H), 5.86 (m, 1H), 5.67 (m, 1H), 4.63 (d, J=5.6 Hz, 1H), 4.26-4.40 (m, 2H), 4.19 (t, J=5.2 Hz, 1H); ¹³C NMR (125 MHz, DMSO-d₆) 167.58, 158.21, 152.10, 150.64, 150.17, 125.44, 124.31, 110.43, 101.51, 77.88, 72.91, 64.71, 58.79. UV (H₂O) λₘₐₓ 280 nm (pH 2), 282 mm (pH 7), 283 nm (pH 11); Anal. Calcd. for (C₁₃H₁₅N₅O₄.0.6H₂O) C, 49.40; H, 5.17; N, 22.16. Found C, 49.75, H, 5.13, N, 21.86.

### Compound 21 (Scheme 3 for 7-Deaza-7-Substituted-Neplanocin A Analogs)

A mixture of compound 19 (183 mg, 0.70 mmol) and acetic anhydride in pyridine was heated to 60° C. for 2 days. The mixture was evaporated and purified by column chromatography on a silica gel to give 20a, 20b (˜260 mg) as a white solid. To a mixture of 20a, 20b (260 mg) in methylene chloride (30 mL) was added H₂SO₄ (0.5 mL) and HNO₃ (Fuming, 0.5 mL) (prepared from the addition of H₂SO₄ into HNO₃) at 0° C. The mixture was stirred at 0° C. for 15 min and neutralized by NaHCO₃ (excess) and MeOH (5 mL). It was filtered through Celite and washed with ethyl acetate. The combined filtrate was purified by column chromatography on a silica gel to give 21 (225 mg, 68%) as a light yellow solid. mp: 66-67° C.; [α]²⁷_{D} −141.94 (c 0.19, CHCl₃); ¹H NMR (500 MHz, CDCl₃) δ 10.69 (br s, 1H), 8.74 (s, 1H), 8.17 (s, 1H), 6.16 (m, 1H), 6.06 (m, 1H), 5.97 (m, 1H), 5.44 (t, J=6.0 Hz, 1H), 4.78 (m, 2H), 2.53 (s, 3H), 2.15 (s, 3H), 2.14 (s, 3H), 2.00 (s, 3H); ¹³C NMR (100 MHz, CDCl₃) 170.29, 170.11, 169.67, 169.56, 153.95, 151.16, 151.12, 142.86, 128.91, 128.73, 128.05, 98.34, 75.84, 73.04, 62.91, 60.25, 26.21, 20.72, 20.61, 20.38. UV (CHCl₃) λₘₐₓ 347, 279 nm; Anal. Calcd. for (C₂₀H₂₁N₅O₉) C, 50.53; H, 5.45; N, 14.73. Found C, 50.23, H, 4.50, N, 14.49.

### Compound 22 (7—Deaza-7-Nitro-Neplanocin A)

A mixture of compound 21 (215 mg) in saturated methanolic ammonia was heated to 80° C. for 16 h. After cooling, the mixture was purified by column chromatography on a silica gel to give 16 (92 mg, 66%) as a yellow solid. mp: 245-246° C.; ¹H NMR (400 MHz, DMSO-d₆) δ 8.43 (s, 1H), 8.23 (s, 1H), 7.80 (br s, 1H), 7.20 (br s, 1H), 5.64 (d, J=2.0 Hz, 1H), 5.59 (m, 1H), 5.13 (d, J=6.8 Hz, 1H), 4.98 (d, J=6.0 Hz, 1H), 4.92 (t, J=5.6 Hz, 1H), 4.41 (t, J=6.0 Hz, 1H), 4.22 (q, J=6.4 Hz, 1H), 4.10 (br s, 2H); ¹³C NMR (100 MHz, CDCl₃) 156.98, 154.49, 152.02, 150.65, 128.98, 128.33, 123.08, 95.44, 77.41, 72.76, 65.69, 59.04. UV (H₂O) λₘₐₓ 339, 261 nm (pH 2), 371, 276 nm (pH 7), 371, 275 nm (pH 11); Anal. Calcd. for (C₁₂H₁₃N₅O₅.0.4H₂O) C, 45.83; H, 4.42; N, 22.27. Found C, 45.81; H, 4.24; N, 22.11.

### Compound 23 (7-Deaza-7-Amino-Neplanocin A)

To a suspension of compound 22 (4 mg) in EtOH/H₂O (1/1, 0.5 mL) was added hydrazine (7 uL) and Raney Ni (excess). The mixture immediately turned to colorless solution. It was filtered through Celite and evaporated to dryness to give compound 23. The compound 23 was slowly decomposed in room temperature. ¹H NMR (500 MHz, CD₃OD) δ 8.02 (s, 1H), 6.55 (s, 1H), 5.81 (s, 1H), 5.61 (s, 1H), 4.60 (d, J=5.5 Hz, 1H), 4.32 (m, 2H), 4.16 (t, J=5.5 Hz, 1H).

### Compound 24 (Scheme 4 for 7-Deaza-7-Substituted-Neplanocin A Analogs)

A mixture of compound 14 (240 mg. 0.35 mmol), tetravinyl tin (158 mg, 0.67 mmol) and (PPh₃)₄Pd (40 mg, 0.035 mmol) in HMPA (2 mL) was stirred at 80° C. for 16 h. The mixture was poured into water and extracted with ethyl acetate. The organic layer was evaporated and purified by column chromatography on a silica gel. The appropriate fraction was collected and evaporated. The residue was treated with saturated methanolic ammonia and heated to 80° C. for 16 h. After cooling, the mixture was purified by column chromatography on a silica gel to give 24 (52 mg, 26%) as a white solid. mp: 98-99° C.; ¹H NMR (500 MHz, CDCl₃) δ 8.66 (s, 1H), 7.4-7.5 (m, 6H), 7.2-7.35 (m, 9H), 6.05 (br s, 1H), 5.94 (br s, 1H), 5.61 (dd, J=18 and 1.5 Hz, 1H), 5.28 (dd, J=18 and 1.5 Hz, 1H), 5.25 (d, J=5.5 Hz, 1H), 4.61 (d, J=6.0 Hz, 1H), 4.04 (dt, J=15.5 and 2 Hz, 1H), 3.86 (d, J=16 Hz, 1H), 1.46 (s, 3H), 1.32 (s, 3H); ¹³C NMR (100 MHz, CDCl₃) 152.27, 151.21, 150.85, 149.83, 143.70, 128.53, 127.96, 127.24, 127.19, 122.67, 122.42, 115.43, 114.67, 114.15, 112.65, 87.33, 84.83, 84.06, 65.06, 61.51, 27.51, 26.06.

### Compound 26 (7-Acetylene-Substituted Isopropylidine Analog)

A mixture of compound 14 (500 mg. 0.73 mmol), trimethylsilyl acetylene (140 mg, 1.43 mmol), CuI (15 mg, 0.073 mmol), triethyl amine (0.20 mL, 1.43 mol) and (PPh₃)₄Pd (42 mg, 0.036 mmol) in DMF (10 mL) was stirred at 60° C. for 3 h. The mixture was poured into water and extracted with ethyl acetate. The organic layer was evaporated and purified by column chromatography on a silica gel. The appropriate fraction was collected and evaporated. The residue was treated with saturated methanolic ammonia and heated to 80° C. for 16 h. After cooling, the mixture was purified by column chromatography on a silica gel to give 26 (340 mg, 82%) as a light yellow solid. mp: 99-100° C.; ¹H NMR (400 MHz, CDCl₃) δ 8.36 (s, 1H), 7.4-7.5 (m, 6H), 7.2-7.35 (m, 9H), 6.98 (s, 1H), 5.99 (s, 1H), 5.83 (s, 1H), 5.60 (br s, 2H), 5.17 (d, J=6 Hz, 1H), 4.55 (d, J=6 Hz, 1H), 4.03 (d, J=15.2, 1H), 3.84 (d, J=16.4 Hz, 1H), 3.27 (s, 1H), 1.43 (s, 3H), 1.30 (s, 3H); ¹³C NMR (100 MHz, CDCl₃) 157.35, 153.36, 149.65, 149.56, 143.77, 128.54, 127.95, 127.22, 126.71, 122.59, 112.54, 103.79, 93.90, 87.30, 84.91, 83.97, 79.93, 77.54, 64.52, 61.48, 27.52, 26.12.

### Compound 29 (Scheme 5 for 7-Deaza-7-Substituted-Neplanocin A Analogs)

A mixture of compound 28 (101 mg, 0.38 mmol) and acetic anhydride (0.25 mL) in pyridine (2 mL) and methylene chloride (2 mL) was stirred at rt overnight. The mixture was evaporated and purified by column chromatography on a silica gel. The appropriate fraction was collected and evaporated and dissolved in CH₂Cl₂/H₂O (10 mL/0.5 mL). It was treated with DDQ (260 mg, 1.14 mmol) and stirred at rt for 3 h. The mixture was mixed with water (20 mL) and extracted with methylene chloride. The combined organic layer was purified by column chromatography on a silica gel to give a reddish sticky liquid. It was dissolved in chloroform and the insoluble solid was filtered off and the filtrate was evaporated to give 29 (80 mg, 78%) as a white solid. mp: 47° C.; [α]²⁴_{D} 24.95 (c 0.45, CHCl₃); ¹H NMR (400 MHz, CDCl₃) δ 6.14 (s, 1H), 5.68 (d, J=6 Hz, 1H), 5.29 (t, J=5.6 Hz, 1H), 4.69 (br s, 3H), 2.13 (s, 3H), 2.10 (s, 6H); ¹³C NMR (100 MHz, CDCl₃) 170.45, 170.06, 139.84, 133.36, 73.14, 72.43, 72.00, 60.15, 20.74, 20.71, 20.63; Anal. Calcd. for (C₁₂H₁₆O₇) C, 52.94, H, 5.92 Found C, 52.88, H, 5.92.

### Compound 30 (Scheme 5 for 7-Deaza-7-Substituted-Neplanocin A Analogs)

To a mixture of compound 29 (80 mg, 0.29 mmol), compound 5 (127 mg, 0.46 mmol) and Ph₃P (199 mg, 0.76 mmol) in THF (10 mL) was added DIAD (154 mg, 0.76 mmol) at 0° C. After stirring 30 min at rt, the mixture was evaporated and purified by column chromatography on a silica gel to give 30 (153 mg, 100%) as a light yellow solid mp: 49° C.; [α]²⁶_{D} −126.83 (c 0.25, CHCl₃); ¹H NMR (400 MHz, CDCl₃) δ 8.62 (s, 1H), 7.35 (s, 1H), 6.11 (m, 1H), 6.03 (m, 1H), 5.95 (dd, J=5.6, 0.8 Hz, 1H), 5.42 (t, J=5.6 Hz, 1H), 4.75 (m, 2H), 2.14 (s, 3H), 2.13 (s, 3H), 2.00 (s, 3H). UV (CHCl₃) λₘₐₓ 308, 269, 240 nm; Anal. Calcd. for (C₁₈H₁₇ClIN₃O₆) C, 40.51; H, 3.21; N, 7.87. Found C, 40.53; H, 3.24; N, 7.65.

### Compound 31 (Scheme 5 for 7-Deaza-7-Substituted-Neplanocin A Analogs)

A mixture of compound 30 (550 mg. 1.03 mmol), tri-n-butyl vinyl tin (0.9 mL, 3.0 mmol), CuI (39 mg, 0.206 mmol), triethyl amine (0.29 mL, 2.0 mol) and (PPh₃)₄Pd (119 mg, 0.10 mmol) in DMF (10 mL) was stirred at 60° C. for 8 h. The mixture was poured into water and extracted with ethyl acetate. The organic layer was evaporated and purified by column chromatography on a silica gel to give 31 (310 mg, 69%) as a light yellow sticky liquid. [α]²⁷_{D} −142.24 (c 0.21, CHCl₃); ¹H NMR (400 MHz, CDCl₃) δ 8.60 (s, 1H), 7.35 (s, 1H), 7.22 (ddd, J=17.6, 12, 0.8 Hz, 1H), 6.15 (m, 1H), 6.05 (m, 1H), 5.97 (dd, J=6, 1.2 Hz, 1H), 5.45 (t, J=5.6 Hz, 1H), 5.29 (dd, J=10.8, 1.2 Hz, 1H), 4.76 (m, 2H), 2.14 (s, 3H), 2.13 (s, 3H), 2.00 (s, 3H); ¹³C NMR (100 MHz, CDCl₃) 170.35, 170.26, 169.74, 152.54, 151.79, 150.78, 140.97, 130.72, 126.91, 121.71, 115.83, 115.29, 114.75, 75.85, 73.00, 61.60, 60.45, 20.75, 20.65, 20.46. UV (CHCl₃)λₘₐₓ 243 nm.

### Compound 32 (Scheme 5 for 7-Deaza-7-Substituted-Neplanocin A Analogs)

A mixture of compound 30 (350 mg. 0.66 mmol), trimethylsilyl acetylene (0.186 mL, 1.32 mmol), CuI (13 mg, 0.066 mmol), triethyl amine (0.184 mL, 1.32 mol) and (PPh₃)₄Pd (38 mg, 0.033 mmol) in DMF (7 mL) was stirred at 60° C. for 3 h. The mixture was poured into water and extracted with ethyl acetate. The organic layer was evaporated and purified by column chromatography on a silica gel to give 32 (190 mg, 57%) as a light yellow solid. mp: 56-57° C.; [α]²⁷_{D}−??? (c ????); ¹H NMR (500 MHz, CDCl₃) δ 8.63 (s, 1H), 7.42 (s, 1H), 6.10 (br s, 1H), 6.02 (br s, 1H), 5.94 (d, J=6 Hz, 1H), 5.40 (t, J=6 Hz, 1H), 4.74 (s, 2H), 2.133 (s, 3H), 2.128 (s, 3H), 1.99 (s, 3H), 0.27 (s, 9H); ¹³C NMR (100 MHz, CDCl₃) 170.61, 170.46, 169.88, 153.85, 151.98, 151.09, 141.68, 130.46, 130.31, 117.46, 99.17, 98.93, 96.03, 76.14, 73.27, 62.25, 60.66, 21.02, 20.92, 20.70, 0.00. UV (???) λₘₐₓ ??? nm; Anal. Calcd. for (C₂₃H₂₆ClN₃O₆Si) C, 54.81; H, 5.20; N, 8.34. Found C, 54.42; H, 5.16; N, 8.44.

### Compound 25 (7-Deaza-7-Vinyl-Substituted Neplanocin A)

A mixture of compound 31 (290 mg), methanol (5 mL), dioxane (3 mL) and liquid ammonia (˜15 mL) was heated to 80° C. for 16 h. After cooling the mixture was purified by column chromatography on a silica gel to give 25 (106 mg, 55%) as a light yellow solid. mp: 168-170° C.; [α]²⁶_{D} −187.84 (c 0.11, MeOH); ¹H NMR (400 MHz, DMSO-d₆) δ 8.03 (s, 1H), 7.32 (s, 1H), 7.09 (dd, J=17.6, 10.8 Hz, 1H), 6.65 (br s, 2H), 5.5-5.6 (m, 3H), 5.0-5.1 (m, 2H), 4.85-4.95 (m, 2H), 4.40 (t, J=5.6 Hz, 1H), 4.05-4.15 (m, 3H); ¹³C NMR (100 MHz, DMSO-d₆) 157.99, 151.77, 151.02, 150.32, 129.63, 124.78, 119.07, 113.93, 112.68, 100.86, 77.62, 72.76, 63.97, 59.02. UV (H₂O) λₘₐₓ 294 nm (pH 2), 287 nm (pH 7), 287 nm (pH 11); Anal. Calcd. for (C₁₄H₁₆N₄O₃.0.15H₂O) C, 57.78; H, 5.65; N, 19.25. Found C, 57.83, H, 5.68, N, 19.14.

### Compound 27 (7-Deaza-7-Acetylene-Neplanocin A)

A mixture of compound 32 (180 mg), methanol (3 mL), dioxane (3 mL) and liquid ammonia (˜10 mL) was heated to 80° C. for 16 h. After cooling the mixture was purified by column chromatography on a silica gel to give 25 (75 mg, 73%) as a light yellow solid. mp: 126-128° C.; [α]²⁶_{D} −194.42 (c 0.10, MeOH); ¹H NMR (400 MHz, CD₃OD) δ 8.12 (s, 1H), 7.37 (s, 1H), 5.80 (m, 1H), 5.63 (m, 1H), 4.58 (d, J=5.6 Hz, 1H), 4.29 (m, 2H), 4.19 (t, J=5.2 Hz, 1H), 3.71 (s, 1H); ¹³C NMR (100 MHz, DMSO-d₆) 157.92, 153.08, 150.83, 149.84, 127.71, 124.17, 102.84, 93.52, 83.20, 78.08, 77.41, 72.77, 64.58, 58.99. UV (H₂O) λₘₐₓ 287 nm (pH 2), 281 nm (pH 7), 279 nm (pH 11); Anal. Calcd. for (C₁₄H₁₄N₄O₃.0.65H₂O.0.5CH₃OH) C, 55.46; H, 5.55; N, 17.84. Found C, 55.48, H, 5.46, N, 17.78.

### Compound 35 (Scheme 6 for 7-Deaza-7-Substituted-Neplanocin A Analogs)

To a mixture of compound 33 (300 mg, 0.71 mmol), compound 34 (211 mg, 0.88 mmol) and Ph₃P (371 mg, 1.41 mmol) in THF (30 mL) was added DIAD (285 mg, 1.41 mmol) at 0° C. After stirring 30 min at rt, the mixture was evaporated and purified by column chromatography on a silica gel. The appropriate fraction was collected and evaporated. The residue was treated with 1M TBAF (1 mL) in THF (30 mL) and stirred at rt for 1 h. The mixture was purified by column chromatography on a silica gel to give 35 (175 mg, 61%) as a white solid. mp: 78-80° C.; [α]²⁶_{D} −208.01 (c 0.14, CHCl₃); ¹H NMR (500 MHz, CDCl₃) δ 8.01 (br s, 1H), 7.10 (d, , J=3.5 Hz, 1H), 6.51 (d, J=3.5 Hz, 1H), 5.76 (d, J=5.5 Hz, 1H), 5.68 (s, 1H), 5.36 (s, 1H), 4.72 (d, J=6.0 Hz, 1H), 4.50 (d, J=14 Hz, 1H), 4.41 (d, J=14 Hz, 1H), 3.5 (br s, 1H), 2.7 (br s, 1H), 1.48 (s, 3H), 1.37 (s, 3H), 1.26 (t, J=6.5 Hz, 6H); ¹³C NMR (125 MHz, CDCl₃) 152.14, 151.36, 150.56, 150.37, 150.01, 128.56, 122.71, 114.83, 112.20, 99.82, 84.56, 84.50, 67.97, 59.65, 36.52, 27.48, 26.04, 19.34, 19.25. UV (CHCl₃) λₘₐₓ 1298, 286, 246 nm.

### Compound 36 (Scheme 6 for 7-Deaza-7-Substituted-Neplanocin A Analogs)

A suspension of compound 35 in aqueous 2N NaOH (10 mL) was refluxed for 16 h. It was neutralized by 1N HCl and evaporated. The mixture was purified by column chromatography on a silica gel to give 36 (82 mg, 60%) as a white solid. mp: 164-166° C.;

[α]²⁷_{D} −74.88 (c 0.12, MeOH); ¹H NMR (500 MHz, CD₃OD) δ 7.94 (s, 1H), 6.59 (d, J=3.5 Hz, 1H), 6.44 (d, J=3.5 Hz, 1H), 5.73 (m, 1H), 5.55 (br s, 1H), 5.37 (d, J=6 Hz, 1H), 4.59 (d, J=5 Hz, 1H), 4.34 (m, 2H), 1.46 (s, 3H), 1.36 (s, 3H); ¹³C NMR (125 MHz, CD₃OD) 160.84, 152.32, 150.96, 150.61, 123.08, 118.15, 111.80, 101.52, 100.36, 85.05, 83.59, 63.99, 58.47, 26.34, 24.72. UV (MeOH) λₘₐₓ 262, 220 nm; Anal. Calcd. for (C₁₅H₁₈N₄O₄.0.7CH₃OH) C, 55.34; H, 6.15; N, 16.44. Found C, 55.50; H, 6.04; N, 16.40.

### Compound 37 (7-Deaza-Guanine Analog)

A mixture of compound 36 (70 mg) and HCl (0.2 mL) in MeOH (20 mL) and water (1 mL) was heated to 50° C. for 3 h. It was neutralized by NaHCO₃ and purified by column chromatography on silica gel to give 37 (35 mg, 57%) as a white solid. mp: 233-234° C.; [α]²⁷_{D} −94.19 (c 0.11, MeOH); ¹H NMR (400 MHz, DMSO d) δ10.26 (s, 1H), 6.54 (d, J=4.5 Hz, 1H), 6.22 (d, J=4.5 Hz, 1H), 6.16 (br s, 2H), 5.52 (m, 1H, 5.32 (m, 1H), 4.84 (br s, 2H), 4.39 (d, J=7 Hz, 1H), 4.06 (m, 2H), 3.98 (t, J=6 Hz, 1H); ¹³C NMR (100 MHz, DMSO-d₆) 158.16, 152.29, 150.79, 145.91, 124.12, 118.01, 102.94, 100.59, 77.94, 72.61, 64.54, 58.98. UV (H₂O) λₘₐₓ 263 nm (pH 2), 262 nm (pH 7), 264 nm (pH 11); Anal. Calcd. for (C₁₂H₁₄N₄O₄.0.8H₂O) C, 49.55; H, 5.34; N, 19.26. Found C, 49.89; H, 5.46; N, 19.12.

### 2′-Deoxy Carbocyclic Cyclopentenyl Nucleosides

### Schemes 7 and 8 for 2′-Deoxy Carbocyclic Cyclopentenyl Nucleosides Analogs

### Compound 43 (Scheme 8 Hydroxyl Protected Cyclopentenyl Uracil Analog)

To a solution of Ph₃P (150 mg, 0.57 mmol) in THF (5 mL) was added DIAD (115 mg, 0.57 mmol) at 0° C. After stirring 30 min, the mixture was cooled to −78° C. and treated with a solution of 42 (produced as per scheme 7) (106 mg, 0.28 mmol) in THF (5 mL). The mixture was stirred for 10 min and treated with N³-benzoyluracil (123 mg, 0.57 mmol) and slowly warmed to rt. It was stirred for 3 h, concentrated in vacuo and purified by column chromatography on a silica gel (EtOAc:Hexane=1:2.5 to 1:1) to give a white solid. It was dissolved in methanol and saturated with ammonia at 0° C. The mixture was stirred at rt for 16 h and purified by column chromatography on a silica gel (EtOAc:Hexane=1:2.5 to 1:1) to give 43 (61 mg, 47%) as a white solid. mp: 172-174° C.; [α]²⁴_{D} −118.0 (c 0.18, MeOH); ¹H NMR (500 MHz, CDCl₃) δ 8.66 (br s, 1H), 7.11 (d, J=7.5 Hz, 1H), 5.76 (d, J=8.0 Hz, 1H), 5.70 (dd, J=7.5 and 2.0 Hz, 1H), 5.56 (s, 1H), 5.20 (t, J=3.5 Hz, 1H), 4.45 (s, 2H), 2.5-2.4 (m, 1H), 2.15-2.05 (m, 1H), 1.2-0.8 (m, 28H); ¹³C NMR (125 MHz, CDCl₃) 163.03, 153.83, 150.84, 140.36, 124.64, 102.51, 73.10, 60.18, 58.07, 40.50, 17.47, 17.41, 17.32, 17.29, 17.19, 17.15, 17.14, 17.12, 13.19, 13.12, 12.55, 12.51. UV (MeOH) λₘₐₓ 267.0 nm; Anal. Calcd. for (C₂₂H₃₈N₂O₅Si₂) C, 56.62; H, 8.21; N, 6.00. Found C, 56.68; H, 8.24; N, 6.02.

### Compound 44 (Scheme 8—Cyclopentenyl Uracil Analog)

A solution of 43 (192 mg, 0.41 mmol) and Et₃N.3HF in THF (10 mL) was stirred at rt for 16 h. It was concentrated in vacuo and the residue was purified by column chromatography on a silica gel (MeOH: CH₂Cl₂=1:8) to give 44 (89 mg, 97%) as a white solid. mp: 149-150° C.; [α]²⁶_{D} −43.09 (c 0.21, MeOH); ¹H NMR (500 MHz, CD₃OD) δ 7.40 (d, J=7.5 Hz, 1H), 5.8-5.7 (m, 2H), 5.68 (d, J=8.0 Hz, 1H), 4.93 (d, J=7.5 Hz, 1H), 4.35 (d, J=15.0 Hz, 1H), 4.29 (d, J=15.5 Hz, 1H), 2.4-2.3 (m, 1H), 2.2-2.1 (m, 1H); ¹³C NMR (125 MHz, CD₃OD) 164.97, 154.44, 151.50, 141.71, 124.30, 101.27, 74.28, 60.25, 58.38, 41.05. UV (H₂O) λₘₐₓ 268.0 nm (ε 9,775, pH 2), 268.0 nm (ε 11,869, pH 7), 266.0 nm (ε 8852, pH 11). Anal. Calcd. for (C₁₀H₁₂N₂O₄) C, 53.57; H, 5.39; N, 12.49. Found C, 52.79, H, 5.27, N, 12.14.

### Compound 45 (Scheme 8—Hydroxyl Protected Cyclopentenyl Cytosine Analog)

To a mixture of compound 43 (200 mg, 0.43 mmol), 2,4,6-triisopropylbenzene sulfonyl chloride (260 mg, 0.86 mmol) and DMAP (53 mg, 0.43 mmol) in anhydrous acetonitrile (15 mL) was added triethyl amine (0.24 mL, 1.71 mmol) at 0° C. The mixture was stirred at rt for 24 h and treated with ammonium hydroxide (30% solution, 5 mL). After stirring at rt for 4 h, the mixture was diluted with water (50 mL) and extracted with ethyl acetate (20 mL×4). The combined organic layer was dried over MgSO₄ and filtered. The filtrate was concentrated in vacuo and the residue was purified by column chromatography on a silica gel (MeOH: CH₂Cl₂=1:20 to 1:15) to give 45 (160 mg, 80%) as a light yellow solid. mp: 218-220° C.; [α]²⁶_{D} −77.87 (c 0.13, CHCl₃); ¹H NMR (500 MHz, CDCl₃) δ 7.16 (d, J=7.0 Hz, 1H), 5.80 (d, J=7.5 Hz, 2H), 5.57 (s, 1H), 5.17 (br s, 1H), 4.44 (t, J=15.3 Hz, 2H), 2.5-2.4 (m, 1H), 2.15-2.05 (m, 1H), 1.2-0.8 (m, 28H); ¹³C NMR (125 MHz, CDCl₃) 165.67, 156.76, 152.70, 141.26, 125.94, 94.90, 73.26, 60.73, 58.23, 41.02, 17.49, 17.45, 17.35, 17.34, 17.23, 17.20, 17.19, 13.19, 13.17, 12.57, 12.56. UV (CHCl₃) λₘₐₓ 283.0 nm; Anal. Calcd. for (C₂₂H₃₉N₃O₄Si₂) C, 56.74; H, 8.44; N, 9.02. Found C, 56.27; H, 8.41; N, 8.66.

### Compound 46 (Scheme 8—Cyclopentenyl Cytosine Analog)

A solution of 45 (120 mg, 0.26 mmol) and conc. HCl (1 mL) in MeOH (10 mL) was stirred at rt for 16 h. It was concentrated in vacuo and the residue was dissolved in NH₄OH (30% solution, 5 mL) and concentrated in vacuo. The residue was purified by column chromatography on a silica gel (MeOH: CH₂Cl₂=1:4) to give 46 as a white solid which was further purified by washing with ethanol (2 mL) and dried to give pure 46 (35 mg, 61%). mp 208-210° C.; [α]²⁶_{D} −30.62 (c 0.13, MeOH); ¹H NMR (500 MHz, D₂O) δ 7.45 (d, J=7.5 Hz, 1H), 5.96 (d, J=8.0 Hz, 1H), 5.69 (d, J=1.5 Hz, 1H), 5.6-5.5 (m, 1H), 4.3-4.2 (m, 2H), 2.3-2.2 (m, 1H), 2.15-2.05 (m, 1H); ¹³C NMR (125 MHz, D₂O) 162.58, 153.92, 152.84, 144.39, 125.37, 95.38, 74.52, 61.78, 58.28, 40.45. UV (H₂O) λₘₐₓ 284.0 nm (ε 13,114, pH 2), 275.0 nm (ε 9,293, pH 7), 275.0 nm (ε 8,937, pH 11). Anal. Calcd. for (C₁₀H₁₃N₃O₃) C, 53.80; H, 5.87; N, 18.82. Found C, 53.39; H, 5.97; N, 18.41.

### Compound 47 (Scheme 8—Hydroxyl Protected Thymine Analog)

To a solution of Ph₃P (351 mg, 1.34 mmol) in THF (10 mL) was added DIAD (0.26 mL, 1.34 mmol) at 0° C. After stirring 30 min, the mixture was cooled to −78° C. and treated with a solution of 42 (250 mg, 0.67 mmol) in THF (10 mL). The mixture was stirred for 10 min and treated with N³-benzoylthymine (308 mg, 1.34 mmol) and slowly warmed to rt. It was stirred for 3 h, concentrated in vacuo and purified by column chromatography on a silica gel (EtOAc:Hexane=1:5.5) to give a white solid. It was dissolved in methanol and saturated with ammonia at 0° C. The mixture was stirred at rt for 16 h and purified by column chromatography on a silica gel (EtOAc:Hexane=1:3 to 1:2) to give 47 (178 mg, 39%) as a light yellow sticky liquid. [α]²⁶_{D} −121.28 (c 0.30, CH₂Cl₂); ¹H NMR (500 MHz, CDCl₃) δ 8.42 (br s, 1H), 6.89 (d, J=1.5 Hz, 1H), 5.77 (d, J=7.5 Hz, 1H), 5.57 (d, J=1.0 Hz, 1H), 5.22 (t, J=3.5 Hz, 1H), 4.46 (s, 2H), 2.5-2.4 (m, 1H), 2.15-2.05 (m, 1H), 1.89 (s, 3H), 1.2-0.8 (m, 28H); ¹³C NMR (125 MHz, CDCl₃) 163.50, 153.27, 150.84, 136.11, 125.27, 111.07, 73.19, 59.75, 58.11, 40.41, 17.46, 17.37, 17.31, 17.26, 17.19, 17.14, 17.13, 17.11, 13.24, 13.20, 12.58, 12.57, 12.47. UV (CH₂Cl₂)λₘₐₓ 273.0 nm.

### Compound 48 (Scheme 8—Cyclopentenyl Thymine Analog)

A solution of 47 (145 mg, 0.30 mmol) and Et₃N.3HF (0.15 mL) in THF (10 mL) was stirred at rt for 16 h. It was concentrated in vacuo and the residue was purified by column chromatography on a silica gel (MeOH: CH₂Cl₂=1:8) to give 48 (63 mg, 88%) as a white solid. mp: 165-167° C.; [α]²⁶_{D} −73.14 (c 0.19, MeOH); ¹H NMR (500 MHz, CD₃OD) δ 7.20 (s, 1H), 5.75 (m, 1H), 5.73 (br s, 1H), 4.93 (d, J=7.0 Hz, 1H), 4.35 (d, J=15.0 Hz, 1H), 4.30 (d, J=15.0 Hz, 1H), 2.4-2.3 (m, 1H), 2.2-2.1 (m, 1H), 1.88 (s, 3H); ¹³C NMR (125 MHz, D₂O) 166.61, 152.35, 152.17, 138.96, 126.07, 111.13, 74.57, 60.63, 58.29, 40.20, 11.32. UV (H₂O) λₘₐₓ 273.0 nm (ε 9,894, pH 2), 273.0 nm (ε 10,954, pH 7), 272.0 nm (ε 9,779, pH 11). Anal. Calcd. for (C₁₁H₁₄N₂O₄) C, 55.46; H, 5.92; N, 11.76. Found C, 55.41, H, 5.88, N, 11.54.

### Compound 49 (Scheme 8—6-Chloropurine Cyclopentenyl Analog)

To a solution of Ph₃P (577 mg, 2.20 mmol) in THF (20 mL) was added DIAD (0.43 mL, 2.20 mmol) at 0° C. After stirring 30 min, the mixture was cooled to −78° C. and treated with a solution of 42 (410 mg, 1.10 mmol) in THF (20 mL). The mixture was stirred for 10 min and treated with 6-chloropurine (340 mg, 2.20 mmol) and slowly warmed to rt. It was stirred for 3 h, concentrated in vacuo and purified by column chromatography on a silica gel (EtOAc:Hexane=1:5) to give a white solid. It was dissolved in THF (20 mL) and treated with Et₃N.3HF (0.4 mL). The mixture was stirred at rt for 16 h and purified by column chromatography on a silica gel (MeOH: CH₂Cl₂=1:8) to give 49 (118 mg, 40%) as a white solid. mp: 168-170° C.; [α]²⁵_{D} −112.5 (c 0.09, MeOH); ¹H NMR (500 MHz, CD₃OD) δ 8.75 (s, 1H), 8.50 (s, 1H), 6.0-5.9 (m, 2H), 5.13 (m, 1H), 4.39 (q, J=15.0 Hz, 2H), 2.6-2.4 (m, 2H); ¹³C NMR (125 MHz, CD₃OD) 154.65, 151.55, 151.41, 149.72, 145.14, 131.30, 123.21, 74.59, 59.29, 58.43, 41.54. UV (MeOH) λₘₐₓ 265.0 nm; Anal. Calcd. for (C₁₁H₁₁ClN₄O₂) C, 49.54, H, 4.16, N, 21.01. Found C, 49.81; H, 4.04; N, 20.88.

### Compound 50 (Scheme 8—Cyclopentenyl Adenine Analog)

A solution of 49 (59 mg, 0.22 mmol) in saturated methanolic ammonia (10 mL) was heated at 80° C. in a steel bomb for 10 h. After the solution was cooled, the solvent was evaporated in vacuo and the residue was purified by column chromatography on a silica gel (MeOH: CH₂Cl₂=1:5) to give 50 (47 mg, 86%) as a white solid. mp: 236-238° C.; [α]²⁶_{D} −107.2 (c 0.20, MeOH); ¹H NMR (500 MHz, CD₃OD) δ 8.23 (s, 1H), 8.06 (s, 1H), 5.96 (s, 1H), 5.81 (m, 1H), 5.07 (br s, 1H), 4.38 (q, J=15.5 Hz, 2H), 2.6-2.4 (m, 2H); ¹³C NMR (125 MHz, DMSO-d₃) 156.45, 155.01, 152.82, 149.73, 139.21, 123.66, 119.64, 74.26, 58.64, 57.83, 42.52. UV (H₂O) λₘₐₓ 260.0 nm (ε 14,535, pH 2), 262.0 nm (ε 14,832, pH 7), 262.0 nm (ε 14,615, pH 11).; Anal. Calcd. for (C₁₁H₁₃N₅O₂) C, 53.43; H, 5.30; N, 28.32. Found C, 53.58; H, 5.38; N, 28.30.

### Compound 51 (Scheme 8—Cyclopentenyl Hypoxanthine Analog)

A solution of 49 (67 mg, 0.25 mmol), mercaptoethanol (68 mg, 0.88 mmol) and sodium methoxide (50 mg, 0.88 mmol) in methanol (10 mL) was refluxed for 10 h. The mixture was concentrated in vacuo and the residue was purified by column chromatography on a silica gel (MeOH: CH₂Cl₂=1:5) to give 51 (50 mg, 80%) as a white solid. mp: 126-127° C.; [α]²⁶_{D} −118.9 (c 0.21, MeOH); ¹H NMR (500 MHz, CD₃OD) δ 8.06 (s, 1H), 8.00 (s, 1H), 5.94 (s, 1H), 5.82 (m, 1H), 5.08 (m, 1H), 4.37 (q, J=15.2 Hz, 2H), 2.6-2.4 (m, 2H); ¹³C NMR (125 MHz, CD₃OD) 157.60, 154.09, 148.55, 144.98, 138.54, 124.19, 123.83, 74.53, 58.70, 58.41, 42.07. UV (H₂O) λₘₐₓ 251.0 nm (ε 11,121, pH 2), 250.0 nm (ε 14,734, pH 7), 254.0 nm (ε 12,375, pH 11).; Anal. Calcd. for (C₁₁H₁₂N₄O₃.H₂O) C, 49.62; H, 5.30; N, 21.04. Found C, 49.40; H, 5.24; N, 20.88.

### Compound 52 (Scheme 8—6-Chloro-2-Amino Purine Cyclopentenyl Analog)

To a solution of Ph₃P (703 mg, 2.68 mmol) in THF (25 mL) was added DIAD (0.52 mL, 2.68 mmol) at 0° C. After stirring 30 min, the mixture was cooled to −78° C. and treated with a solution of 42 (500 mg, 1.34 mmol) in THF (25 mL). The mixture was stirred for 10 min and treated with 2-amino-6-chloropurine (455 mg, 2.68 mmol) and slowly warmed to rt. It was stirred for 16 h, concentrated in vacuo and purified by column chromatography on a silica gel (EtOAc:Hexane=1:5) to give a white solid. It was dissolved in THF (20 mL) and treated with Et₃N.3HF (0.5 mL). The mixture was stirred at rt for 16 h and purified by column chromatography on a silica gel (MeOH: CH₂Cl₂=1:8) to give 52 (107 mg, 28%) as a white solid. mp: >200° C. (dec); [α]²⁶_{D} −51.2 (c 0.21, MeOH); ¹H NMR (500 MHz, DMSO-d₆) δ 7.93 (s, 1H), 6.89 (br s, 2H), 5.72 (br s, 1H), 5.49 (m, 1H), 5.05 (d, J=6.5 Hz, 1H), 4.86 (m, 2H), 4.2-4.1 (m, 2H), 2.3-2.1 (m, 2H); ¹³C NMR (125 MHz, DMSO-d₆) 160.11, 155.52, 154.19, 149.73, 141.45, 124.20, 123.04, 74.19, 58.62, 57.83, 42.28. UV (MeOH) λₘₐₓ 311.0 nm; Anal. Calcd. for (C₁₁H₁₂ClN₅O₂) C, 46.90; H, 4.29; N, 24.86. Found C, 46.67, H, 4.25, N, 24.56.

### Compound 53 (Scheme 8—Cyclopentenyl Guanine Analog)

A solution of 52 (107 mg, 0.38 mmol), mercaptoethanol (148 mg, 1.90 mmol) and sodium methoxide (108 mg, 1.90 mmol) in methanol (10 mL) was refluxed for 24 h. After cooling, the mixture was neutralized by acetic acid (0.5 mL) and concentrated in vacuo and the residue was purified by column chromatography on a silica gel (MeOH: CH₂Cl₂=1:2) to give a white solid which was washed with methanol to give 53 (65 mg, 60%) as a white solid. mp: >250° C. (dec); [α]²⁶_{D} −47.7 (c 0.16, MeOH); ¹H NMR (500 MHz, DMSO-d₆) δ 10.59 (br s, 1H), 7.52 (s, 1H), 6.48 (br s, 2H), 5.73 (s, 1H), 5.41 (m, 1H), 5.06 (d, J=5.5 Hz, 1H), 4.89 (m, 2H), 4.2-4.1 (m, 2H), 2.3-2.1 (m, 2H); ¹³C NMR (125 MHz, DMSO-d₆) 157.28, 154.95, 153.94, 151.32, 135.31, 123.67, 117.37, 74.15, 58.61, 57.23, 42.81. UV (H₂O), λₘₐₓ 254.0 nm (ε 10,510, pH 2), 253.0 nm (ε 12,429, pH 7), 255.0 nm (ε 11,492, pH 11); Anal. Calcd. for (C₁₁H₁₃N₅O₃.H₂O) C, 46.97; H, 5.38; N, 24.90. Found C, 46.52, H, 5.34, N, 24.51.

### (2′-Deoxy-2′-Fluoro Carbocyclic Cyclopentenyl Nucleosides-Scheme 9 and 10)

Compound 28 (Scheme 9—Cyclopentenyl analog)

To a solution of compound 1 (6.00 g, 14.0 mmol) in DMF (60 mL) was added 60% dispersion of sodium hydride (1.12 g, 28 mmol) at 0° C. After 10 min, the mixture was treated with 4-methoxy benzylchloride (2.1 mL, 15.4 mmol) and stirred at rt for 3 h. It was poured into water (300 mL) and extracted with ethyl ether (100 mL×2). The combined ether layer was dried (MgSO₄) and evaporated. The resulting solid was dissolved in MeOH (150 mL), THF (150 mL) and water (15 mL) and treated with conc. HCl (0.5 mL). The mixture was heated to 60° C. for 8 h and neutralized by NaHCO₃. After evaporation, the mixture was purified by column chromatography on a silica gel to give 53 (2.61 g, 70%) as a white solid. mp: 69-70° C.; ¹H NMR (400 MHz, CD₃OD) δ 7.31 (d, J=8.4 Hz, 2H), 6.88 (d, J=8.8 Hz, 2H), 5.8 (m, 1H), 4.65 (d, J=11.2 Hz, 1H), 4.52 (d, J=11.2 Hz, 1H), 4.15-4.35 (m, 5H); ¹³C NMR (100 MHz, CD₃OD) 159.45, 148.76, 130.33, 129.35, 125.35, 113.29, 79.89, 73.20, 71.62, 71.09, 58.59, 54.23.

### Compound 54 (Scheme 9—Tritylated Cyclopentenyl Analog)

A mixture of compound 53 (0.50 g, 1.88 mmol), trityl chloride (574 mg, 2.06 mmol), DMAP (11 mg, 0.094 mmol) and triethyl amine (0.39 mL, 2.82 mmol) in methylene chloride (20 mL) was stirred at rt for 16 h. It was washed with brine and purified by column chromatography on a silica gel to give 54 (908 g, 95%) as a white solid. mp: ????° C.; ¹H NMR (400 MHz, CDCl₃) δ 7.4-7.5 (m, 6H), 7.2-7.35 (m, 11H), 6.9-6.95 (m, 2H), 6.03 (d, J=1.6 Hz, 1H), 4.66 (d, J=11.2 Hz, 1H), 4.59 (d, J=11.2 Hz, 1H), 4.37 (m, 1H), 4.23 (m, 2H), 3.90 (d, J=14.4 Hz, 2H), 3.81 (s, 3H), 3.73 (d, J=14.8 Hz, 1H), 3.02 (d, J=5.6 Hz, 1H), 2.55 (d, J=8.8 Hz, 1H); ¹³C NMR (100 MHz, CDCl₃) 159.53, 147.67, 143.86, 129.73, 129.68, 128.56, 127.89, 127.08, 125.36, 113.97, 86.91, 79.88, 74.61, 71.99, 70.38, 61.41, 55.31.

### Compound 55 (Scheme 9—Tritylated-3-O-Protected Cyclopentenyl Analog)

A mixture of compound 54 (908 mg, 1.78 mmol), triethyl orthoformate (0.63 mL) and CAN (74 mg) in methylene chloride (30 mL) was stirred at rt for 2 h. It was cooled to −78° C. and treated with DIBAL-H (3.3 mL, 18 mmol) and stirred 1 h at −78° C. It was slowly warmed to 0° C. and quenched with MeOH. The mixture was evaporated and treated 100 mL of ethyl acetate/MeOH (20/1) and stirred 1 h. The mixture was filtered through Celite and washed with ethyl acetate. The combined organic layer was purified by column chromatography on a silica gel to give 55 (340 mg, 34%) as a sticky liquid.: ¹H NMR (400 MHz, CDCl₃) δ 7.4-7.5 (m, 6H), 7.2-7.35 (m, 11H), 6.9-6.95 (m, 2H), 6.11 (d, J=1.6 Hz, 1H), 4.6-4.75 (m, 4H), 4.2-4.3 (m, 3H), 3.86 (d, J=14.4 Hz, 1H), 3.80 (s, 3H), 3.63 (d, J=14 Hz, 1H), 3.38 (q, J=6.8 Hz, 2H), 2.93 (d, J=8 Hz, 1H), 1.01 (t, J=7.2 Hz, 1H); ¹³C NMR (100 MHz, CDCl₃) 159.32, 145.31, 143.87, 130.39, 129.65, 128.54, 127.87, 127.07, 113.82, 94.86, 86.79, 79.43, 78.75, 71.96, 71.08, 63.46, 61.50, 55.29, 14.98.

### Compound 56 (Scheme 9—Tritylated-3-O-Protected-2-Fluoro-Cyclopentenyl Analog)

To a solution of compound 55 (4.2 g, 7.4 mmol) and pyridine (2.9 g, 37 mmol) in methylene chloride (150 mL) was added triflic anhydride (1.88 mL, 11.1 mmol) at 0° C. After 15 min, the reaction mixture was washed with brine and evaporated. The resulting oil was dissolved in acetonitrile (150 mL) and treated with 1M solution of TBAF (11.1 mL) and stirred at rt for 1 h. After evaporation, the mixture was purified by column chromatography on a silica gel to give 56 (3.2 g, 76%) as a sticky liquid.: ¹H NMR (400 MHz, CDCl₃) δ 7.4-7.5 (m, 6H), 7.2-7.4 (m, 11H), 6.90 (d, J=8.4 Hz, 2H), 5.96 (s, 1H), 5.03 (dt, J=52.8, 3.6 Hz, 1H), 4.5-4.7 (m, 6H), 3.83 (s, 1H), 3.81 (s, 3H), 3.3-3.6 (m, 3H), 1.08 (t, J=7.2 Hz, 1H); ¹³C NMR (100 MHz, CDCl₃) 159.33, 143.80, 142.53, 142.47, 129.87, 129.65, 128.51, 127.88, 127.09, 125.41, 125.35, 113.86, 106.75, 104.86, 94.54, 86.85, 83.49, 83.23, 82.78, 82.54, 71.22, 63.45, 60.64, 55.30, 14.95; ¹⁹F NMR (376 MHz, CDCl₃)-187.77 (dt, J=53, 18.4 Hz).

### Compound 57 (Scheme 9—Tritylated-3-O-Protected-2-Fluororo Cyclopentenyl Analog)

A mixture of compound 56 (3.2 g, 5.6 mmol) and DDQ (1.92 g, 8.4 mmol) in CH₂Cl₂/H₂O (150 mL/7 mL) was stirred at rt for 3 h. The mixture was washed with brine and purified by column chromatography on a silica gel to give a reddish sticky liquid. It was purified by column chromatography on a silica gel one more time to give 57 (2.15 mg, 86%) as a colorless sticky liquid. [α]²⁶_{D} 25.81 (c 0.41, CHCl₃); ¹H NMR (400 MHz, CDCl₃) δ 7.4-7.5 (m, 6H), 7.2-7.35 (m, 9H), 6.04 (s, 1H), 4.87 (dt, J=52, 3.2 Hz, 1H), 4.5-4.8 (m, 4H), 3.81 (d, J=14 Hz, 1H), 3.61 (d, J=14.4 Hz, 1H), 3.45 (m, 2H), 2.22 (d, J=7.6 Hz, 1H), 1.10 (t, J=7.2 Hz, 1H); ¹³C NMR (100 MHz, CDCl₃) 143.76, 142.90, 142.86, 128.51, 127.96, 127.91, 127.14, 106.70, 104.82, 94.12, 86.94, 82.74, 82.47, 77.84, 77.57, 63.58, 60.64, 14.88; ¹⁹F NMR (376 MHz, CDCl₃)-188.90 (dt, J=63, 30 Hz); Anal. Calcd. for (C₂₈H₂₉FO₄) C, 74.98, H, 6.52 Found C, 74.57, H, 6.50.

### As Per Scheme 10-(2′-Deoxy-2′-Fluoro Carbocyclic Cyclopentenyl Nucleosides)

### Compound 58 (Scheme 10—Protected-2′-Fluoro-Cyclopentenyl Uracil Analog)

To a mixture of compound 57 (300 mg, 0.67 mmol), N³-benzoyluracil (207 mg, 1.34 mmol) and Ph₃P (351 mg, 1.34 mmol) in THF (30 mL) was added DIAD (0.26 mL, 1.34 mmol) at 0° C. After stirring 30 min at rt, the mixture was evaporated and purified by column chromatography on a silica gel. The appropriate fraction was collected and evaporated. The residue was treated with saturated methanolic ammonia and stirred at rt for 16 h. The mixture was purified by column chromatography on a silica gel to give 58 (202 mg, 56%) as a white solid. mp: 84-85° C.; [α]²⁶_{D} −46.23 (c 0.13, CHCl₃); ¹H NMR (400 MHz, CDCl₃) 8.58 (br s. 1H), 7.4-7.5 (m, 6H), 7.2-7.35 (m, 9H), 7.00 (dd, J=8.4, 2 Hz, 1H), 5.85-5.95 (m, 2H), 5.71 (dd, J=8, 3 Hz, 1H), 5.09 (dd, J=51, 5 Hz, 1H), 46-4.7 (m, 3H), 3.7-4.0 (m, 2H), 3.4-3.5 (m, 2H), 1.11 (t, J=6.8 Hz, 3H); ¹³C NMR (125 MHz, CDCl₃) 163.42, 151.18, 147.57, 147.55, 143.62, 142.19, 142.17, 128.52, 128.04, 127.34, 123.84, 101.70, 94.90, 93.83, 92.30, 87.36, 83.83, 83.61, 63.99, 61.11, 60.46, 60.30, 60.17, 14.98; ¹⁹F NMR (376 MHz, CDCl₃)-195.71 (dt, J=50, 16 Hz); UV (CHCl₃) λₘₐₓ 264.0 nm.

### Compound 59 (Scheme 10—Protected-2′-Fluoro-Cyclopentenyl Cytosine Analog)

To a mixture of compound 58 (225 mg, 0.41 mmol), 2,4,6-triisopropylbenzene sulfonyl chloride (251 mg, 0.83 mmol) and DMAP (51 mg, 0.41 mmol) in anhydrous acetonitrile (15 mL) was added triethyl amine (0.23 mL, 1.66 mmol) at 0° C. The mixture was stirred at rt for 24 h and treated with ammonium hydroxide (30% solution, 5 mL). After stirring at rt for 4 h, the mixture was evaporated and purified by column chromatography on a silica gel to give 59 (168 mg, 76%) as a white solid. mp: 115-116° C.; [α]²⁶_{D} 60.75 (c 0.13, CHCl₃); ¹H NMR (500 MHz, CDCl₃) 7.4-7.5 (m, 6H), 7.2-7.35 (m, 9H), 7.12 (dd, J=7, 2 Hz, 1H), 6.06 (dd, J=15, 2.5 Hz, 1H), 5.94 (s, 1H), 5.73 (d, J=7.5 Hz, 1H), 5.13 (dd, J=51, 5 Hz, 1H), 4.6-4.7 (m, 3H), 3.7-4.0 (m, 2H, 3.4-3.5 (m, 2H), 1.09 (t, J=7.5 Hz, 3H); ¹³C NMR (125 MHz, CDCl₃) 165.77, 156.66, 146.32, 146.30, 143.70, 143.62, 128.54, 128.00, 127.28, 125.37, 94.85, 93.80, 93.58, 92.07, 87.24, 83.89, 83.67, 63.87, 61.21, 61.16, 61.07, 14.99; ¹⁹F NMR (470 MHz, CDCl₃)-195.84 (dt, J=51, 15 Hz); UV (CHCl₃)λₘₐₓ 280.0 nm.

### Compound 60 (Scheme 10—Deprotected-2′-Fluoro-Cyclopentenyl Cytosine Analog)

A mixture of compound 59 (160 mg) and HCl (0.3 mL) in MeOH (15 mL), THF (15 mL) and water (1 mL) was heated to 50° C. for 16 h. It was neutralized by NaHCO₃ and purified by column chromatography on silica gel to give 10 (62 mg, 87%) as a white solid. mp: 205-206° C.; [α]²⁴_{D} 65.37 (c 0.24, MeOH); ¹H NMR (500 MHz, CD₃OD) 7.44 (d, J=7 Hz, 1H), 5.94 (d, J=8 Hz, 1H), 5.9 (m, 1H), 5.81 (s, 1H), 5.04 (ddd, J=51.5, 5.5, 1 Hz, 1H), 4.80 (d, J=17.5 Hz, 1H), 4.3-4.4 (m, 2H); ¹³C NMR (100 MHz, DMSO-d₆) 166.14, 156.31, 151.74, 151.70, 143.40, 122.37, 97.44, 95.57, 93.55, 77.98, 77.71, 60.24, 60.07, 58.46; ¹⁹F NMR (376 MHz, DMSO-d₆)-195.81 (ddd, J=51, 18, 13 Hz); UV (H₂O) λₘₐₓ 282 nm (pH 2), 272 nm (pH 7), 272 nm (pH 11); Anal. Calcd. for (C₁₀H₁₂FN₃O₃.0.2CH₃OH)C, 49.47, H, 5.21, N, 16.97. Found C, 49.58; H, 5.24; N, 16.92.

### Compound 61 (Scheme 10—Protected-2′-Fluoro-Cyclopentenyl Thymine Analog)

To a mixture of compound 57 (264 mg, 0.59 mmol), N³-benzoylthymine (198 mg, 1.18 mmol) and Ph₃P (310 mg, 1.18 mmol) in THF (30 mL) was added DIAD (239 mg, 1.18 mmol) at 0° C. After stirring 30 min at rt, the mixture was evaporated and purified by column chromatography on a silica gel. The appropriate fraction was collected and evaporated. The residue was treated with saturated methanolic ammonia and stirred at rt for 16 h. The mixture was purified by column chromatography on a silica gel to give 61 (150 mg, 46%) as a white solid. mp: 85-86° C.; [α]²⁷_{D} 34.68 (c 0.12, CHCl₃); ¹H NMR (500 MHz, CDCl₃) 8.9 (br s, 1H), 7.4-7.5 (m, 6H), 7.2-7.35 (m, 9H), 6.90 (s, 1H), 5.98 (s, 1H), 5.91 (m, 1H), 5.10 (dd, J=51, 5.5 Hz, 1H), 4.6-4.75 (m, 3H), 3.7-4.0 (m, 2H), 3.4-3.5 (m, 2H), 1.95 (s, 3H), 1.11 (t, J=7 Hz, 3H); ¹³C NMR (125 MHz, CDCl₃) 163.89, 163.86, 151.21, 151.19, 150.01, 147.12, 147.10, 143.60, 137.90, 137.88, 128.51, 128.04, 127.97, 127.37, 127.23, 124.27, 110.06, 94.90, 93.97, 92.45, 87.35, 83.86, 83.64, 63.98, 61.13, 60.04, 59.91, 14.98, 12.64; ¹⁹F NMR (470 MHz, CDCl₃)-195.93 (dt, J=51, 15 Hz); UV (CHCl₃) λₘₐₓ 269 nm.

### Compound 62 (Scheme 10—Deprotected-2′-Fluoro-Cyclopentenyl Thymine Analog)

A mixture of compound 61 (160 mg) and HCl (0.3 mL) in MeOH (15 mL), THF (15 mL) and water (1 mL) was heated to 50° C. for 16 h. It was neutralized by NaHCO₃ and purified by column chromatography on silica gel to give 10 (65 mg, 88%) as a white solid. mp: 174-175° C.; [α]²⁵_{D} 23.73 (c 0.13, MeOH); ¹H NMR (400 MHz, CD₃OD) 7.16 (br s, 1H), 5.77 (br s, 1H), 5.72 (m, 1H), 4.9-5.1 (m, 1H), 4.78 (d, J=18 Hz, 1H), 4.80 (d, J=17.5 Hz, 1H), 4.2-4.4 (m, 2H), 1.85 (s, 3H); ¹³C NMR (100 MHz, CD₃OD) 165.16, 151.75, 151.41, 151.36, 138.60, 121.93, 109.09, 96.76, 94.86, 78.17, 77.91, 59.87, 59.70, 58.27, 10.84; ¹⁹F NMR (376 MHz, CD₃OD)-198.07 (ddd, J=50, 17, 11 Hz); UV (H₂O) λₘₐₓ 271 nm (pH 2), 271 nm (pH 7), 269 nm (pH 11); Anal. Calcd. for (C₁₁H₁₃FN₂O₄.0.2CH₃OH) C, 51.22, H, 5.30, N, 10.67. Found C, 51.15; H, 5.16; N, 10.70.

### Compound 63 (Scheme 10—Protected-2′-Fluoro-Cyclopentenyl Adenine Analog)

To a mixture of compound 57 (100 mg, 0.22 mmol), 6-chloropurine (68 mg, 0.44 mmol) and Ph₃P (115 mg, 0.44 mmol) in THF (10 mL) was added DIAD (89 mg, 0.44 mmol) at 0° C. After stirring 30 min at rt, the mixture was evaporated and purified by column chromatography on a silica gel. The appropriate fraction was collected and evaporated. The residue was treated with saturated methanolic ammonia and heated to 80° C. for 16 h. After cooling, the mixture was purified by column chromatography on a silica gel to give 63 (77 mg, 62%) as a white solid. mp: 178-180° C.; [α]²⁷_{D} 15.91 (c 0.11, CHCl₃); ¹H NMR (400 MHz, CDCl₃) 8.39 (s, 1H), 7.77 (d, J=1.6 Hz, 1H), 7.4-7.5 (m, 6H), 7.2-7.35 (m, 9H), 6.15 (s, 1H), 6.01 (m, 1H), 5.74 (br s, 2H), 5.18 (ddd, J=51, 5.2, 2 Hz, 1H), 4.87 (d, J=14.8 Hz, 1H), 4.71 (d, J=7.2 Hz, 1H), 4.64 (d, J=7.2 Hz, 1H), 3.96 (d, J=15.2 Hz, 1H), 3.83 (d, J=15.2 Hz, 1H), 3.46 (m, 2H), 1.10 (t, J=7.2 Hz, 3H); ¹³C NMR (100 MHz, CDCl₃) 155.39, 153.07, 150.30, 147.16, 147.12, 143.62, 139.96, 128.49, 128.00, 127.29, 123.76, 119.53, 94.89, 94.64, 92.70, 87.27, 83.61, 83.36, 63.88, 60.97, 57.76, 57.59, 14.95; ¹⁹F NMR (470 MHz, CDCl₃)-195.93 (dt, J=51, 15 Hz); UV (CHCl₃) λₘₐₓ 260 nm; Anal. Calcd. for (C₃₃H₃₂FN₅O₃) C, 70.07; H, 5.70; N, 12.38. Found C, 69.61; H, 5.58; N, 12.01.

### Compound 64 (Scheme 10—Deprotected-2′-Fluoro-Cyclopentenyl Adenine Analog)

A mixture of compound 63 (250 mg) and HCl (0.6 mL) in MeOH (30 mL), THF (30 mL) and water (2 mL) was heated to 50° C. for 16 h. It was neutralized by NaHCO₃ and purified by column chromatography on silica gel to give crude 64. Recrystallization from MeOH/H₂O gave pure 64 (90 mg, 77%) as a white solid. mp: 255-256° C.; [α]²⁷_{D} −39.14 (c 0.17, H₂O); ¹H NMR (400 MHz, DMSO-d₆) 8.13 (s, 1H), 7.92 (s, 1H), 7.23 (br s, 1H), 5.82 (s, 1H), 5.72 (br s, 1H), 5.68 (d, J=6.4 Hz, 1H), 4.9-5.2 (m, 2H), 4.8-4.9 (m, 1H), 4.1-4.2 (m, 2H); ¹³C NMR (100 MHz, DMSO-d₆) 156.41, 152.96, 152.76, 152.70, 150.22, 139.96, 120.31, 119.19, 98.07, 96.14, 77.61, 77.36, 58.39, 56.70, 56.53; UV (H₂O) λₘₐₓ 260 nm (pH 2), 260 nm (pH 7), 260 nm (pH 11); Anal. Calcd. for (C₁₁H₁₂FN₅O₂) C, 49.81, H4.56, N, 26.40. Found C, 49.93; H, 4.62; N, 26.19.

### Compound 65 (Scheme 10—Protected-2′-Fluoro-Cyclopentenyl Guanine Analog)

To a mixture of compound 57 (300 mg, 0.67 mmol), N-isobutanoyl-2-amino-6-chloropurine (240 mg, 1.00 mmol) and Ph₃P (351 mg, 1.34 mmol) in THF (30 mL) was added DIAD (271 mg, 1.34 mmol) at 0° C. After stirring 30 min at rt, the mixture was evaporated and purified by column chromatography on a silica gel. The appropriate fraction was collected and evaporated. The residue was treated with 2-mercaptoethanol (144 mg, 1.84 mmol) and sodium methoxide (25 wt %, 0.40 mL, 1.84 mmol) in methanol and refluxed for 4 h. The mixture was neutralized by acetic acid and evaporated. The residue was purified by column chromatography on a silica gel to give 65 (202 mg, 51%) as a white solid. mp: 197-198° C.; [α]²⁷_{D} 37.24 (c 0.11, CH₃OH); ¹H NMR (500 MHz, DMSO-d₆). 10.66 (s, 1H), 7.60 (s, 1H), 7.25-7.45 (m, 15H), 6.49 (br s, 2H), 6.26 (s, 1H), 5.59 (m, 1H), 5.15-5.25 (m, 1H), 4.98 (d, J=14 Hz, 1H), 4.65 (d, J=6.5 Hz, 1H), 4.60 (d, J=6.5 Hz, 1H), 3.79 (d, J=16 Hz, 1H), 3.69 (d, J=15.5 Hz, 1H), 3.3-3.4 (m, 2H), 0.99 (t, J=7 Hz, 3H); ¹³C NMR (125 MHz, DMSO-d₆) 157.21, 154.10, 151.90, 146.42, 146.36, 144.05, 136.70, 128.61, 128.54, 127.68, 123.50, 117.00, 95.53, 94.71, 93.98, 86.91, 83.63, 83.43, 63.31, 61.04, 56.79, 56.65, 15.34; ¹⁹F NMR (470 MHz, DMSO-d₆)-197.1˜−196.9 (m); UV (CH₃OH) λₘₐₓ 253, 210 nm.

### Compound 66 (Scheme 10—Deprotected-2′-Fluoro-Cyclopentenyl Guanine)

A mixture of compound 65 (190 mg) and HCl (0.6 mL) in MeOH (30 mL), THF (30 mL) and water (2 mL) was heated to 50° C. for 16 h. It was neutralized by NaHCO₃ and purified by column chromatography on silica gel to give crude 66. Recrystallization from MeOH/H₂O gave pure 66 (65 mg, 71%) as a white solid. mp: 194-196° C.; [α]²⁷_{D} −28.97 (c 0.10, H₂O); ¹H NMR (400 MHz, DMSO-d₆) 8.8 (br s, 1H), 7.46 (d, J=0.8 Hz, 1H), 6.49 (br s, 2H), 5.76 (s, 1H), 5.68 (br s, 1H), 5.44 (br s, 1H), 4.7-5.1 (m, 3H), 4.0-4.2 (m, 2H), 4.80 (d, J=17.5 Hz, 1H), 4.2-4.4 (m, 2H), 1.85 (s, 3H); ¹³C NMR (125 MHz, DMSO-d₆) 157.28, 154.12, 152.64, 152.59, 151.90; 136.39, 120.42, 117.02, 97.87, 96.33, 77.36, 77.16, 58.37, 56.38, 56.24; ¹⁹F NMR (376 MHz, DMSO-d₆)-197.78 (ddd, J=52, 16, 5 Hz); UV (H₂O) λₘₐₓ 254 nm (pH 2), 251 nm (pH 7), 267 nm (pH 11); Anal. Calcd. for (C₁₁H₁₂FN₅O₃.1.2H₂O) C, 43.62, H, 4.79, N, 23.12. Found C, 43.69; H, 4.70; N, 23.12.

### As Per Schemes 11 and 12-(3′-Deoxy-Carbocyclic Cyclopentenyl Nucleosides)

### Compound 67 (Scheme 11—Protected Cyclopentenyl Analog)

To a mixture of compound 53 (2.10 g, 7.9 mmol) and imidazole (1.63 g, 24 mmol) in CH₂Cl₂ (100 mL) was added TBDPSCl (2.28 g, 8.3 mmol) at 0° C. and stirred at rt for 3 h. The mixture was washed with brine and evaporated and purified by column chromatography on a silica gel to give 67 (3.79 g, 95%) as a colorless liquid. ¹H NMR (500 MHz, CDCl₃) δ 7.6-7.7 (m, 4H), 7.3-7.45 (m, 8H), 6.86-6.92 (m, 2H), 5.89 (m, 1H), 4.63 (d, J=11.5 Hz, 1H), 4.57 (d, J=11 Hz, 1H), 4.2-4.5 (m, 5H), 3.81 (s, 3H), 1.06 (s, 9H); ¹³C NMR (125 MHz, CDCl₃) 159.54, 149.68, 135.57, 135.55, 133.30, 133.28, 129.81, 129.80, 129.77, 129.72, 127.76, 125.04, 113.98, 79.77, 74.17, 71.93, 70.71, 61.41, 55.34, 26.86, 19.30; Anal. Calcd. for (C₃₀H₃₆O₅Si) C, 71.39, H, 7.19 Found C, 71.35, H, 7.29.

### Compound 68 (Scheme 11—Protected Cyclopentenyl Epoxide Analog)

To a solution of compound 67 (3.79 g, 7.5 mmol) and trimethyl orthoacetate (1.26 mL, 9.75 mmol) in CH₂Cl₂ (150 mL) was added 1M solution of TMSCl (9.75 mL) at 0° C. The mixture was stirred at rt for 1 h and evaporated. The residue was dissolved in MeOH (150 mL) and treated with K₂CO₃ (2.07 g, 15 mmol) and stirred at rt for 3 h. The mixture was poured into water (300 mL) and extracted with ethyl ether (100 mL×3). The combined organic layer was evaporated and purified by column chromatography on a silica gel to give 68 (2.60 g, 70%) as a colorless liquid. ¹H NMR (400 MHz, CDCl₃) δ 7.65-7.7 (m, 4H), 7.3-7.45 (m, 8H), 6.86-6.92 (m, 2H), 6.68 (m, 1H), 4.57 (s, 2H), 4.51 (m, 1H), 4.25-4.45 (m, 2H), 3.80 (s, 3H), 3.56-3.7 (m, 2H), 1.05 (s, 9H); ¹³C NMR (100 MHz, CDCl₃) 159.37, 146.89, 135.53, 133.12, 133.10, 130.12, 129.82, 129.81, 129.66, 128.69, 127.78, 113.84, 79.93, 71.98, 61.46, 56.46, 55.28, 52.18, 26.74, 19.24.

### Compound 69 (Scheme 11—Protected-3′-Deoxy Cyclopentenyl Analog)

To a solution of compound 68 (2.60 g, 5.34 mmol) ethyl ether (150 mL) was added LiAlH₄ (200 mg, 5.3 mmol) at 0° C. The mixture was stirred at rt for 1 h and quenched with water. The mixture was extracted with ethyl ether (100 mL×2). The combined organic layer was evaporated and purified by column chromatography on a silica gel to give 69 (2.40 g, 92%) as a colorless liquid.: ¹H NMR (400 MHz, CDCl₃) δ 7.6-7.7 (m, 4H), 7.3-7.5 (m, 8H), 6.86-6.94 (m, 2H), 5.78 (m, 1H), 4.57 (s, 2H), 4.3-4.44 (m, 2H), 4.24 (d, J=15.2 Hz, 1H), 4.15 (d, J=15.2 Hz, 1H), 3.81 (s, 3H), 2.94 (d, J=6 Hz, 1H), 2.2-2.5 (m, 2H), 1.06 (s, 9H); ¹³C NMR (100 MHz, CDCl₃) 159.37, 147.35, 135.50, 133.39, 133.36, 130.14, 129.71, 129.59, 127.71, 122.03, 113.89, 82.33, 71.67, 70.63, 63.01, 55.29, 39.88, 26.77, 19.27.

### Compound 70 (Scheme 11—5′-O-Protected Cyclopentenyl Analog)

A mixture of compound 69 (2.4 g, 4.9 mmol), benzoyl chloride (0.85 mL, 7.4 mmol) in pyridine (10 mL) and CH₂Cl₂ (50 mL) was stirred at rt for 5 h. It was washed with 1N HCl and purified by column chromatography. The appropriate fraction was collected and evaporated. The residue was treated with DDQ (1.72 g, 7.5 mmol) in CH₂Cl₂ (150 mL) and water (7 mL). The mixture was stirred at rt for 30 min and washed with water. The organic phase was evaporated and purified by column chromatography. The appropriate fraction was collected and evaporated. The residue was dissolved in MeOH (100 mL) and treated with sodium methoxide (0.35 g) and stirred at rt for 30 min. The mixture was evaporated and purified by column chromatography on a silica gel to give 70 (1.30 g, 72%) as a colorless liquid.: ¹H NMR (400 MHz, CDCl₃) δ 7.6-7.7 (m, 4H), 7.25-7.5 (m, 6H), 5.76 (m, 1H), 4.56 (m, 1H), 4.30 (m, 1H), 4.25 (d, J=15.2 Hz, 1H), 4.18 (d, J=15.2 Hz, 1H), 2.45-2.55 (m, 1H), 2.2-2.3 (m, 1H), 1.07 (s, 9H); ¹³C NMR (100 MHz, CDCl₃) 147.21, 135.53, 133.33, 129.79, 127.74, 124.34, 75.61, 71.23, 62.87, 39.27, 26.80, 19.24.

### Compound 71 (Scheme 11—Protected Azido-Substituted Cyclopentenyl Analog)

To a mixture of compound 70 (1.3 g, 3.5 mmol) and triethyl amine (2.4 mL, 17.5 mmol) in CH₂Cl₂ (50 mL) was added SOCl₂ (0.39 mL, 5.3 mmol) at 0° C. After 30 min, the mixture was washed with water and evaporated and purified by column chromatography. The appropriate fraction (barely separable diastereomeric mixture) was collected and evaporated. The residue was dissolved in DMF (40 mL) and treated with NaN₃ (455 mg, 7.0 mmol) and stirred at rt for 3 h. The mixture was poured into water (150 mL) and extracted with ethyl ether (100 mL×2). The combined organic phase was evaporated and purified by column chromatography. The appropriate fraction was collected and evaporated. The residue was dissolved in CH₂Cl₂ (100 mL) and treated with imidazole (476 mg, 7.0 mmol) and TBDMSCl (633 mg, 4.2 mmol). The mixture was stirred at rt for 12 h and washed with water. The organic phase was evaporated and purified by column chromatography on a silica gel to give 71a and 71b (1.50 g, 67%) as a colorless liquid which was inseparable mixture (71a/71b=4/1).: IR (cm⁻¹) 2095; Anal. Calcd. for (C₂₈H₄₁N₃O₂Si₂) C, 66.22; H, 8.14; N, 8.27. Found C, 66.44, H, 8.18, N, 8.22.

### Scheme 12—(3′-Deoxy-Carbocyclic Cyclopentenyl Nucleosides)

### Compound 72 (Scheme 12—Triazole-Substituted-Protected-Cyclopentenyl Analog)

To a mixture of compound 71 (300 mg, 0.59 mmol) and methyl propiolate (50.4 mg, 0.59 mmol) in tert-BuOH (2 mL) and water (2 mL) was added sodium ascorbate (23 mg, 0.12 mmol) and CuSO₄ (2 mg, 0.012 mmol). The mixture was heated to 70° C. for 16 h. After cooling, the mixture was evaporated and purified by column chromatography on a silica gel to give 72 (260 mg, 74%) as a colorless liquid. ¹H NMR (500 MHz, CDCl₃) δ 8.00 (s, 1H), 7.65-7.7 (m, 4H), 7.35-7.5 (m, 6H), 5.72 (br s, 1H), 5.52 (br s, 1H), 4.44 (m, 1H), 4.28 (s, 2H), 3.97 (s, 3H), 2.68 (dd, J=16.5, 7 Hz, 1H), 2.28-2.36 (m, 1H), 1.07 (s, 9H), 0.83 (s, 9H), −0.07 (s, 3H), −0.09 (s, 3H); ¹³C NMR (125 MHz, CDCl₃) 161.48, 149.24, 140.11, 135.68, 133.29, 133.28, 130.13, 130.11, 128.02, 126.12, 119.05, 80.16, 74.68, 62.83, 52.40, 40.70, 26.97, 25.83, 19.43, 18.13, −4.84, −4.95. Anal. Calcd. for (C₃₂H₄₅N₃O₄Si₂) C, 64.94, H, 7.66, N, 7.10. Found C, 64.29; H, 7.71; N, 7.01.

### Compound 73 (Scheme 12—Deprotected Triazole-Substituted-Cyclopentenyl Analog)

A mixture of compound 72 (300 mg, 0.51 mmol) and Et₃N.3HF (0.5 mL) in THF (10 mL) was heated to 60° C. for 16 h. After cooling, the mixture was evaporated and purified by column chromatography on a silica gel to give 73 (110 mg, 91%) as a white solid. mp: 100-102° C.; [α]²⁷_{D} −179.57 (c 0.16, CH₃OH); ¹H NMR (400 MHz, CD₃OD) δ 8.50 (s, 1H), 5.72 (m, 1H), 5.52 (m, 1H), 4.51 (m, 1H), 4.21 (s, 2H), 3.90 (s, 3H), 2.85-2.95 (m, 1H), 2.35-2.45 (m, 1H); ¹³C NMR (100 MHz, CD₃OD) 160.96, 150.33, 139.10, 126.73, 118.82, 78.00, 74.34, 60.20, 51.08, 39.48. UV (CH₃OH) λₘₐₓ 213 mL Anal. Calcd. for (C₁₀H₁₃N₃O₄) C, 50.21; H, 5.48; N, 17.56. Found C, 50.00; H, 5.46; N, 17.56.

### Compound 74 (Scheme 12—Amid-Substituted Triazole Containing Cyclopentenyl Analog)

A mixture of compound 73 (100 mg, 0.42 mmol) in saturated methanolic ammonia was stirred at rt for 16 h. The mixture was evaporated and purified by column chromatography on a silica gel to give 74 (90 mg, 95%) as a white solid. mp: 146-148° C.; [α]²³_{D} −179.74 (c 0.14, CH₃OH); ¹H NMR (400 MHz, CD₃OD) δ 8.32 (s, 1H), 5.72 (m, 1H), 5.52 (m, 1H), 4.50 (m, 1H), 4.21 (s, 2H), 2.88 (dd, J=16.8, 7.2 Hz, 1H), 2.34-2.44 (m, 1H); ¹³C NMR (100 MHz, CD₃OD) 163.34, 150.13, 142.36, 124.57, 118.95, 78.09, 74.15, 60.22, 39.47. UV (H₂O) λₘₐₓ 211 nm (pH 2), 209 nm (pH 7), 224 nm (pH 11); Anal. Calcd. for (C₉H₁₂N₄O₃) C, 48.21, H, 5.39, N, 24.99. Found C, 47.98; H, 5.37; N, 24.84.

### Compound 75 (Nucleoside Precursory Compound of Scheme 12)

A mixture of compound 71 (100 mg, 0.20 mmol) and PPh₃ (77 mg, 0.30 mmol) in MeOH (5 mL) was refluxed for 1 h. After cooling, it was evaporated to dryness and dissolved in benzene (5 mL). A mixture of methoxy acrylic acid (80 mg, 0.79 mmol) and oxalyl chloride (78 μL, 0.87 mmol) was stirred at rt for 1 h. It was evaporated and dissolved in benzene and treated with AgOCN (237 mg, 1.58 mmol). The mixture was refluxed for 1 h and cooled to rt. The liquid of the suspension was taken by syringe and added to the benzene solution of amine compound. The resulting mixture was stirred at rt for 12 h and evaporated. The residue was purified by column chromatography on a silica gel to give 75 (32 mg, 26%) as a colorless liquid. ¹H NMR (400 MHz, CDCl₃) δ 9.81 (s, 1H), 8.67 (d, J=8.8 Hz, 1H), 7.6-7.7 (m, 4H), 7.35-7.5 (m, 6H), 5.57 (m, 1H), 5.39 (d, J=12.8 Hz, 1H), 4.83 (m, 1H), 4.24 (m, 1H), 4.16 (s, 2H), 3.71 (s, 3H), 2.55 (dd, J=16, 7.2 Hz, 1H), 2.1-2.2 (m, 1H), 1.06 (s, 9H), 0.87 (s, 9H), 0.06 (s, 3H), 0.05 (s, 3H).

### Synthesis of 3-Deazaneplanocin A and 3-Deazaguanosine-3′-deoxy carbocyclic cyclopentenyl nucleosides Schemes 1-3

### Experiment

General: NMR spectra were recorded on 400 or 500 MHz Fourier transform spectrometer; Optical rotations were measured by a Jasco DIP-370 digital polarimeter. High-resolution mass spectra (HRMS) were recorded on a Micromass Autospec high-resolution mass spectrometer using electrospray ionization (ESI) in positive mode. Infrared spectrum was recorded on an Avatar 360 FT-IR as neat type. Melting points were taken on Mel-Temp II melting point apparatus and were uncorrected. TLC was performed on 0.25 mm silica gel. Purifications were carried out using silica gel (60 Å, 32-63 mm) or C₁₈ reversed silica gel (230-400 mesh).

### The Synthesis of 4-Chloro-Imidazo[4,5-c]Pyridine (7) from 4-Amino-2-Chloropyridine 2-Chloro-4-Nitroaminopyridine

4-Amino-2-chloropyridine (10.0 g, mol) was carefully added to 70 mL of concentrated sulfuric acid at 0° C. with ice bath. To the suspension was added 70 mL of 70% nitric acid dropwise while the inside temperature was maintained below 10° C. with salt-ice bath. The reaction mixture was stirred for 1 h at room temperature and then poured onto 300.0 g of crushed ice and allowed to cool to 10° C. for 12 h. The cream-colored precipitate was filtered and washed well with ice water. The aqueous layer was treated with pallet NaOH until the pH of the solution reached at 3. The white solid was separated from the aqueous solution and filtered. The collected solid was dried to give 13.0 g of 2-Chloro-4-nitroaminopyridine in 85% yield. mp 163-164° C.; UV (H₂O) λₘₐₓ 299.0 (ε 11880, pH 11), ¹H-NMR (DMSO, 500 MHz) δ 8.42 (d, J=5.5, 1H), 7.53 (d, J=2.0, 1H), 7.40 (dd, J=5.5, 2.0, 1H), 2.52 (s, 1H); ¹³C-NMR (DMSO, 125 MHz) δ 151.51, 151.29, 145.89, 112.49, 112.26.

### 4-Amino-2-chloro-3-nitropyridine and 4-amino-2-Chloro-5-nitropyridine

2-Chloro-4-nitroaminopyridine (10.0 g, mol) was carefully dissolved in 100 mL of concentrated sulfuric acid at room temperature and heated 100° C. for 1 h. After the solution was cooled to room temperature, it was poured onto 250 g of crushed ice and treated with concentrated ammonium hydroxide until pH was reached at 3 while the temperature was kept below 20° C. with ice bath. The yellow solid was separated and extracted with ethyl acetate (200 ml×3) from aqueous layer. The collected organic layer was concentrated and the residue was purified with silica gel column chromatography (hexane:EtOAc=4:1 to EtOAc v/v) to give 6.0 g of 4-Amino-2-chloro-3-nitropyridine in 70% yield and 2.0 g of 4-amino-2-Chloro-5-nitropyridine in 25% yield. 4-Amino-2-chloro-3-nitropyridine: mp 179-181° C.; UV (H₂O) λₘₐₓ 238.0 (ε 13586, pH 11), ¹H-NMR (DMSO, 500 MHz) δ 7.91 (d, J=6.0, 1H), 7.37 (s, 2H), 6.83 (d, J=6.0, 1H); ¹³C-NMR (DMSO, 125 MHz) δ 149.54, 149.24, 142.69, 142.33, 122.45; 4-amino-2-chloro-5-nitropyridine: ¹H-NMR (DMSO, 500 MHz) δ 8.85 (s, 1H), 7.37 (s, 2H), 6.96 (s, 1H).

### 2-Chloro-3,4-diaminopyridine

4-Amino-2-chloro-3-nitropyridine (6.0 g, 34.57 mmol) in 150 mL of ethanol was hydrogenated over Raney nickel catalyst (6.0 g wet) for 4 h at room temperature under 1.0 atm of H₂ atmosphere. After addition of 4.0 g of celite to the solution, the mixture was stirred vigorously and filtered over celite pad. The filtrate was concentrated and purified with silica gel column chromatography (CH₂Cl₂:MeOH=20:1 v/v) to give 2-Chloro-3,4-diaminopyridine (4.72 g, 32.84 mmol) in 95% yield. ¹H-NMR (DMSO, 500 MHz) δ 7.31 (d, J=5.0, 1H), 6.45 (d, J=5.0, 1H), 5.79 (s, 2H), 4.68 (s, 2H); ¹³C-NMR (DMSO, 125 MHz) δ 143.41, 138.03, 135.61, 126.66, 108.73.

### 4-Chloro-imidazo[4,5-c] Pyridine (14)

2-Chloro-3,4-diaminopyridine (4.70 g, 32.74 mmol) was refluxed in a 1:1 mixture of ethyl orthoformate: acetic anhydride (60 mL) for 5 h. The excess reagents were removed under reduced pressure and the residue was treated with 20 mL of water. The suspension warmed on steam bath and 10% sodium hydroxide was added at 0° C. until pH of the solution remained at 9. The solid was extracted with CH₂Cl₂ (100 mL×5), concentrated and purified with silica gel column chromatography (CH₂Cl₂:MeOH=40:1 to 10:1 v/v) to give compound 14 (4.28 g, 27.83 mmol) in 85% yield. UV (H₂O) λₘₐₓ 274.0 (ε 2455, pH 11), 267.0 (ε 5134 pH 2); NMR (DMSO, 500 MHz) δ 13.27 (br, 1H), 8.50 (s, 1H), 8.13 (d, J=6.0, 1H), 7.64 (d, J=6.0, 1H); ¹³C-NMR (DMSO, 125 MHz) δ 145.11, 142.33, 141.11, 108.20; HRMS (ES) calcd for C₄H₄ClN₄ (M+H⁺) 154.0172, found 154.0174.

### 1-[(1R,4R,5S)-3-(Trityloxymethyl)-4,5-O-iso-propylidinene-2-cyclopenten-1-yl]-4-chloroimidazo[4,5-c]pyridine (8) and 9

To a solution of Ph₃P (1.66 g, 6.30 mmol) in 10 mL of anhydrous THF, 3.80 mL of DIAD was added at 0° C. under N₂ atmosphere. After 30 min, a solution of compound 5 (0.90 g, 2.10 mmol) in 10 mL of anhydrous THF was added to the reaction mixture at −78° C. and additional stirring the mixture for 30 min. Compound 7 (3.20 g, 9.57 mmol) was added to the suspension at −78° C., then the reaction mixture was allowed to stir for 24 h at room temperature. The yellow solution was absorbed on silica gel and purified on silica gel column chromatography (hexane:EtOAc=2:1 to 1:1 v/v) to give products 8 and 9 (2:1) in quantitative yield (1.30 g, 2.30 mmol) including a little impurity from DIAD. The mixture was purified by silica gel column chromatography (hexane:EtOAc=2:1) to give compound 8 (0.15 g, mmol) and 9 (0.13 g, mmol) and a mixture of 8 and 9 (0.92 g). Compound 8: ¹H-NMR (CDCl₃, 500 MHz) δ 8.22 (d, J=11.0 Hz, 1H), 7.86 (s, 1H), 7.65 (d, J=11.0 Hz, 1H), 7.49 (m, 6H), 7.34-7.26 (m, 9H), 6.26 (s, 1H), 6.16 (s, 1H), 5.12 (d, J=5.5 Hz, 1H), 4.64 (d, J=5.5 Hz, 1H), 4.12 (d, J=15.5 Hz, 1H), 3.89 (d, J=15.5 Hz, 1H), 1.44 (s, 3H), 1.31 (s, 3H); ¹³C-NMR (CDCl₃, 125 MHz) δ 151.99, 151.65, 144.63, 143.84, 141.57, 134.34, 128.73, 128.21, 127.51, 121.48, 115.12, 112.90, 87.57, 85.15, 83.69, 66.30, 61.41, 27.75, 26.37; HRMS (ES) calcd for C₃₄H₃₀ClN₃O₃ (M+H⁺) 564.2055, found 564.2062.

Compound 9: ¹H-NMR (CDCl₃, 500 MHz) δ 8.23 (d, J=6.0 Hz, 1H), 7.89 (s, 1H), 7.48 (m, 6H), 7.49 (m, 6H), 7.34-7.26 (m, 9H), 6.14 (s, 1H), 5.45 (s, 1H), 5.19 (d, J=5.5 Hz, 1H), 4.56 (d, J=5.5 Hz, 1H), 4.08 (d, J=15.5 Hz, 1H), 3.93 (d, J=15.5 Hz, 1H), 1.47 (s, 3H), 1.32 (s, 3H); ¹³C-NMR (CDCl₃, 125 MHz) δ 151.04, 143.77, 143.38, 142.64, 141.87, 139.67, 138.59, 128.70, 128.20, 127.53, 121.36, 113.27, 105.86, 87.63, 84.76, 83.94, 67.06, 61.50, 27.51, 25.99.

### 1-[(1R,4R,5S)-3-(Hydroxymethyl)-4,5-dihydroxy-2-cyclopenten-1-yl]-4-chloroimidazo[4,5-c]pyridine (10)

The solution of compound 8 (0.26 g, 0.46 mmol) in 10 mL of MeOH was bubbled with dried HCl at 0° C. for 20 min. The acidic solution was allowed to stir for 4 h at room temperature. After removal of the solvent and excess HCl under reduced pressure, the residue was purified by silica gel column chromatography (CH₂Cl₂:MeOH=10:1 v/v) to give compound 10 (0.12 g, 0.42 mmol) in 92% yield. ¹H-NMR (CD₃OD, 500 MHz) δ 8.52 (s, 1H), 8.24 (d, J=5.5 Hz, 1H), 7.76 (d, J=5.5 Hz, 1H), 6.29 (d, J=1.5 Hz, 1H), 6.05 (d, J=1.5 Hz, 1H), 5.73 (d, J=5.5 Hz, 1H), 4.43 (m, 2H), 4.35 (t, J=5.5 Hz, 1H); ¹³C-NMR (CD₃OD, 125 MHz) δ 151.57, 150.97, 145.88, 140.61, 134.07, 128.38, 123.55, 114.27, 78.82, 73.10, 66.48, 58.88; HRMS (ES) calcd for C₁₂H₁₂ClN₃O₃ (M+H⁺) 282.0646, found 282.0639.

### 3-[(1R,4R,5S)-3-(Hydroxymethyl)-4,5-dihydroxy-2-cyclopenten-1-yl]aminoimidazo[4,5-c]pyridine (11)

The solution of compound 9 (0.10 g, 0.18 mmol) in 10 mL of MeOH was bubbled with dried HCl at 0° C. for 20 min. The acidic solution was allowed to stir for 4 h at room temperature. After removal of the solvent and excess HCl under reduced pressure, the residue was purified by silica gel column chromatography (CH₂Cl₂:MeOH=10:1 v/v) to give product (0.05 g, 0.18 mmol) in 98% yield. The product (40.0 mg, 0.14 mmol) was dissolved in anhydrous hydrazine (10 mL) under N₂ atmosphere followed by refluxing the reaction mixture for 2 h. The resulting solution was diluted with EtOH (10 mL) and concentrated under reduced pressure to give a solid residue. The resulting solid was dissolved with degassed water (20 mL) and treated with Raney-nickel (0.12 g) at room temperature under N₂ atmosphere. The suspension was refluxed for 30 min and then filtered and washed with hot water (10 ml×3) and acidified with 10% HCl (1.0 mL). The acidic solution was concentrated in vacuo and purified on reverse silica gel column using water eluent to give compound 11 (25 mg, 0.084 mmol) 60% yield. UV (H₂O) λₘₐₓ 286.0, 212 (pH 7.4); [α]²⁷_{D} 82.81 (c 0.51, H₂O); ¹H-NMR (CD₃OD, 400 MHz) δ 8.47 (s, 1H), 8.19 (d, J=6.0 Hz, 1H), 7.71 (d, J=6.0 Hz, 1H), 6.24 (s, 1H), 5.99 (d, J=2.0 Hz, 1H), 4.67 (d, J=5.5 Hz, 1H), 4.37 (m, 2H), 4.29 (t, J=5.5 Hz, 1H); ¹³C-NMR (CD₃OD, 100 MHz) δ 151.50, 150.89, 145.81, 140.52, 133.99, 128.32, 123.44, 114.17, 78.74, 72.99, 66.38, 58.77.

### 7H-Imidazo[4,5-c]tetrazolo[1,5-a]pyridine (12)

To a suspension solution of compound 7 (4.20 g, 27.35 mmol) in 20 mL of DMF-[emim]BF₄ (10:1 v/v), LiN₃ (28 mL, 20 wt % in H₂O) was added at room temperature and the reaction mixture was heated at 80° C. for 6 h. The mixture was concentrated under reduced pressure followed by the residue was treated with acetone (20 mL). The result solid was filtered, washed with acetone (10 mL×3) and dried under reduced pressure for 24 h to give a white solid (4.35 g, 26.80 mmol) in 98% yield. mp 205-207° C.; UV (H₂O) λₘₐₓ 271.0 (ε 5134 pH 2); ¹H-NMR (DMSO-d₆, 500 MHz) δ 9.07 (d, J=7.0 Hz, 1H), 8.56 (s, 1H), 7.71 (d, J=7.0 Hz, 1H); ¹³C-NMR (DMSO-d₆, 125 MHz) δ 144.06, 143.83, 134.99, 125.67, 121.22, 106.93; IR (neat) 3074, 2928, 2854, 2361, 1647, 1523 cm⁻¹; HRMS (ES) calcd for C₆H₄N₆ (M+H⁺) 161.0576, found 161.0538.

### 4-Amino-imidazo[4,5-c]pyridine (13)

To a solution of compound 12 (2.75 g, 14.0 mmol) in 50 mL of 6 N HCl was added Pd/C (2.50 g, 10 wt % activated carbon). The black suspension was stirred for 10 min under N₂ atmosphere and then shacked at 3 atm of H₂ atmosphere for 36 h. The reaction mixture was treated with a celite (4.0 g), filtered on a celite pad and washed with degassed water (50 mL×3). The acidic solution was concentrated followed by the residue was dried in vacuo for 48 h to give a white solid 13 (3.36 g, 13.90 mmol) in 99% yield. mp 260-262° C.; NMR (CD₃OD, 500 MHz) δ 8.36 (s, 1H), 7.67 (d, J=7.0 Hz, 1H), 7.20 (d, J=7.0 Hz, 1H); ¹³C-NMR (CD₃OD, 125 MHz) δ 148.71, 143.08, 140.18, 128.74, 125.55, 100.31; HRMS (ES) calcd for C₆H₆N₄ (M+H⁺) 135.0670, found 135.0648.

### 4-(N⁶,N⁶-Di-tert-butyloxycarbonylamino)-1-tert-butyloxycarbonyl-imidazo[4,5-c]pyridine (14)

To a solution of compound 13 (3.30 g, 15.93 mmol) and DMAP (4.87 g, 39.83 mmol) in 50 mL of anhydrous THF was added (Boc)₂O (17.38 g, 79.65 mmol) under N₂ atmosphere at 0° C. After stirring the suspension for 24 at room temperature, the solvent was removed under reduced pressure and the residue was purified on silica gel coluum chromatography (hexane:EtOAc=1:4 to 1:2 v/v) to give compound 14 (6.57 g, 15.13 mmol) in 95% yield. UV (H₂O) λₘₐₓ 264.0 (ε 5134 pH 11), 264.0 (ε 5134 pH 7.4); ¹H-NMR (CDCl₃, 500 MHz) δ 8.45 (d, J=6.0 Hz, 1H), 8.44 (s, 1H), 7.85 (d, J=6.0 Hz, 1H), 1.72 (s, 9H), 1.40 (s, 18H); ¹³C-NMR (CDCl₃, 125 MHz) δ 150.91, 147.15, 144.87, 143.53, 142.61, 138.38, 136.78, 109.59, 87.01, 82.93, 28.00, 27.85; HRMS (ES) calcd for C₂₁H₃₀N₄O₆ (M+Na⁺) 457.2063, found 457.2050.

### 4-(N⁶-tert-Butyloxycarbonylamino)-imidazo[4,5-c]pyridine (15a)

To a solution of compound 14 (2.40 g, 5.52 mmol) in 200 mL of THF, 2.60 mL of NaOMe (12.0 mmol, 25 wt % in MeOH) was added at 0° C. After 10 min, the basic solution was treated with 1.0 N HCl to get pH 9. The resulting solution was treated with MgSO₄ and filtered. The filtrate was concentrated and purified on silica gel pad (CH₂Cl₂ to CH₂Cl₂:MeOH 20:1 v/v) to give compound 15a (1.25 g, 5.32 mmol) in 96% yield. ¹H-NMR (CDCl₃, 500 MHz) δ 11.92 (br, 1H), 11.10 (br, 1H), 8.12 (d, J=6.0 Hz, 1H), 8.15 (s, 1H), 7.48 (d, J=6.0 Hz, 1H), 1.59 (s, 9H); ¹³C-NMR (CDCl₃, 125 MHz) δ 154.23, 150.73, 142.39, 139.43, 139.32, 120.01, 110.59, 82.05, 28.35; HRMS (ES) calcd for C₁₁H₁₄N₄O₂ (M+H⁺) 235.1195, found 235.1168.

### 4-(N6,N⁶-Di-tert-butyloxycarbonylamino)-imidazo[4,5-c]pyridine (15b)

The solution of compound 14 (6.50 g, 14.97 mmol) in 200 mL of MeOH was treated with 15 mL of saturated NaHCO₃ at room temperature. After 1 h, the solvent was removed in vacuo while the temperature of bat was maintained at 25° C. The residue was resolved in 100 mL of cold CH₃Cl and then poured into ice water (200 mL). The organic layer was separated from water layer and the aqueous layer was washed with CH₃Cl (50 mL×3). The collected organic layer was dried over MgSO₄ and concentrated. The residue was purified on silica gel pad (hexane:EtOAc=1:4 to 1:2 v/v) to give compound 15b (4.52 g, 13.47 mmol) in 90% yield. ¹H-NMR (CDCl₃, 500 MHz) δ 13.00 (s, 1H), 8.32 (d, J=6.0 Hz, 1H), 8.31 (s, 1H), 7.62 (d, J=6.0 Hz, 1H), 1.35 (s, 18H); ¹³C-NMR (CDCl₃, 125 MHz) δ 151.57, 143.61, 143.28, 140.56, 140.05, 136.14, 108.29, 83.33, 27.78; HRMS (ES) calcd for C₁₆H₂₂N₄O₄ (M+Na⁺) 357.1539, found 357.1550.

### 1-[(1R,4R,5S)-3-(Trityloxy(ethyl)-4,5-O-iso-propyldinene-2-cyclopenten-1-yl]-4-(N⁶-tert-butyloxycarbonylamino)-imidazo[4,5-c]pyridine (17a) and 16a

The procedure of Mitsunobu reaction of compound 15a with 5 was the same as compound 15b. Compound 17a: ¹H-NMR (CDCl₃, 500 MHz) δ 8.54 (s, 1H), 8.28 (d, J=5.5 Hz, 1H), 7.81 (s, 1H), 7.49 (m, 6H), 7.30 (m, 6H), 7.24 (m, 4H), 7.11 (d, J=5.5 Hz, 1H), 6.14 (s, 1H), 5.43 (s, 1H), 5.19 (d, J=5.0 Hz, 1H), 4.56 (d, J=5.0 Hz, 1H), 4.06 (d, J=15.0 Hz, 1H), 3.91 (d, J=15.0 Hz, 1H), 1.56 (s, 9H), 1.46 (s, 3H), 1.31 (s, 3H); ¹³C-NMR (CDCl₃, 125 MHz) δ 150.98, 150.34, 144.97, 143.64, 141.47, 140.65, 138.72, 130.28, 128.52, 128.02, 127.32, 121.62, 112.87, 101.67, 87.39, 84.57, 83.82, 80.83, 66.52, 61.37, 28.32, 27.39, 25.89; HRMS (ES) calcd for C₃₉H₄₀N₄O₅ (M+H⁺) 645.3088, found 645.3076.

Compound 16a: ¹H-NMR (CDCl₃, 500 MHz) δ 8.36 (d, J=5.5 Hz, 1H 1H), 7.82 (s, 1H), 7.72 (d, J=5.5 Hz, 1H), 7.47 (m, 6H), 7.33 (m, 6H), 7.27 (m, 3H), 6.07 (s, 1H), 5.55 (s, 1H), 5.16 (d, J=6.0 Hz, 1H), 4.55 (d, J=6.0 Hz, 1H), 4.08 (d, J=15.5 Hz, 1H), 3.90 (d, J=15.5 Hz, 1H), 1.45 (s, 9H), 1.41 (s, 3H), 1.29 (s, 3H).

### (−)-1-[(1R,4R,5S)-3-(Trityloxymethyl)-4,5-iso-propylidinene-2-cyclopenten-1-yl]-4-(N⁶,N⁶-di-tert-butyloxycarbonylamino)-imidazo[4,5-c]pyridine (17b) and 16b

To a solution of Ph₃P (6.04 g, 22.98 mmol) in 20 mL of anhydrous THF, 3.80 mL of DIAD was added at 0° C. under N₂ atmosphere. After 30 min, a solution of compound 5 (3.30 g, 7.66 mmol) in 20 mL of anhydrous THF was added to the reaction mixture at −78° C. and additional stirring the mixture for 30 min. N⁶,N⁶-diBoc-3-deazaadenine (15b, 3.20 g, 9.57 mmol) was added to the suspension at −78° C., then the reaction mixture was allowed to stir for 48 h at room temperature. The yellow solution was absorbed on silica gel and purified on silica gel column chromatography (hexane:EtOAc=2:1 to 1:2 v/v) to give compound 17b (4.56 g, 6.13 mmol) in 80% yield and 16b (0.46 g, 0.62 mmol) in 8% yield. Compound 17b: UV (H₂O)λₘₐₓ 269.0 (ε 2455, pH 11), 267.0 (ε 5134 pH 7.4); [α]²⁷_{D} −11.64 (c 1.00, CHCl₃); NMR (CDCl₃, 500 MHz) δ 8.36 (d, J=6.0 Hz, 1H), 7.87 (s, 1H), 7.47 (d, J=7.5 Hz, 6H), 7.42 (d, J=6.0 Hz, 1H), 7.34-7.25 (m, 9H), 6.15 (s, 1H), 5.45 (s, 1H), 5.19 (d, J=5.5 Hz, 1H), 4.59 (d, J=5.5 Hz, 1H), 4.04 (d, J=15.0 Hz, 1H), 4.90 (d, J=15.0 Hz, 1H), 1.46 (s,3H), 1.45 (s, 18H), 1.32 (s, 3H); ¹³C-NMR (CDCl₃, 125 MHz) δ 151.44, 150.47, 144.75, 143.64, 142.34, 141.14, 140.18, 137.30, 128.55, 128.05, 121.58, 112.98, 106.12, 87.41, 84.56, 83.85, 82.98, 66.66, 61.32, 27.98, 27.36, 25.85; IR (neat) 3060, 2973, 2926, 1783, 1748, 1608, 1485, 1450 cm⁻¹; HRMS (ES) calcd for C₄₄H₄₈N₄O₇ (M+H⁺) 745.3597, found 745.3553.

Compound 16b: UV (H₂O) λₘₐₓ 278.0 (ε 2455, pH 11), 277.0 (ε 5134 pH 7.4); [α]²⁷_{D} +25.77 (c 0.98, CHCl₃); NMR (CDCl₃, 400 MHz) δ 8.36 (d, J=5.6 Hz, 1H), 7.83 (s, 1H), 7.72 (d, J=5.6 Hz, 1H), 7.48 (d, J=7.2 Hz, 6H), 7.35-7.25 (m, 9H), 6.07 (s, 1H), 5.55 (s, 1H), 5.16 (d, J=6.0 Hz, 1H), 4.56 (d, J=6.0 Hz, 1H), 4.09 (d, J=16.0 Hz, 1H), 3.90 (d, J=16.0 Hz, 1H), 1.46 (s, 9H), 1.42 (s, 3H), 1.39 (s, 9H), 1.29 (s, 3H); ¹³C-NMR (CDCl₃, 100 MHz) δ 151.96, 151.55, 150.99, 150.54, 143.74, 143.63, 140.69, 137.48, 128.51, 127.99, 127.47, 127.32, 121.81, 115.64, 112.83, 87.40, 85.09, 83.81, 83.41, 83.27, 66.54, 61.33, 28.02, 27.94, 27.68, 26.51; IR (neat) 3055, 2980, 2926, 1784, 1750, 1608, 1488, 1449 cm⁻¹.

### (−)-1-[(1R,4R,5S)-3-(Hydroxymethyl)-4,5-dihydroxy-2-cyclopenten-1-yl]-4-aminoimidazo[4,5-c]pyridine Hydrochloride (3-Deazaneplanocin A, 2)

The solution of a little crude compound 17b (3.50 g, 4.72 mmol) in 100 mL of MeOH was bubbled with dried HCl at 0° C. for 20 min. The acidic solution was allowed to stir for 6 h at room temperature. After removal of the solvent and excess HCl under reduced pressure, to the residue was added 50 mL of water and 50 mL of EtOAc, followed by separation of organic layer from water layer. The aqueous layer was washed with EtOAc (50 mL×3) and concentrated in vacuo to give compound 2 as sticky foam. The product was solved with 40 mL of MeOH-EtOH (3:1 v/v), followed by evaporation of the solvents in vacuo to give a pale yellow solid as HCl salt form (1.26 g, 4.23 mmol) in 90% yield. mp 166-168° C.; UV (H₂O) λₘₐₓ 265.0, 216 (ε 2455, 2222, pH 11), 264.0, 212 (ε 5132, 4034, pH 7.4), 266, 216 (ε 7180,pH 2); [α]²⁷_{D} −100.76 (c 0.12, H₂O); ¹H-NMR (D₂O, 500 MHz) δ 8.31 (s, 1H), 7.65 (d, J=7.0 Hz, 1H), 7.20 (d, J=7.0 Hz, 1H), 6.08 (d, J=1.5 Hz, 1H), 5.54 (m, 1H), 4.69 (d, J=5.0 Hz, 1H), 4.40 (s, 2H), 4.35 (t, J=5.0 Hz, 1H); ¹³CδNMR(D₂O, 125 MHz) δ 152.25, 150.67, 146.70, 142.72, 131.65, 128.73, 127.03, 102.51, 80.63, 75.19, 69.06, 61.56; HRMS (ES) calcd for C₁₂H₁₄N₄O₃ (+M⁺) 263.1145, found 263.1152.

### The Synthesis of Carbocyclic 3-DEAZAGUANOSIN (4)

### N-(4-Oxo-4,5-dihydro-1H-imidazolo[4,5-c]pyridine-6-yl-isobutyramide (23)

A solution 3-deazaguanine (2.0 g, 13.30 mmol) in 50 mL of anhydrous dimethyl acetamide, isobutyricanhydride (5.52 mL, 33.30 mmol) was added at room temperature under N₂ atmosphere. The reaction mixture was stirred for 2 h at 150° C. The mixture was cooled to room temperature and then the solvent was removed under reduced pressure. The residue was purified using silica gel chromatography (CH₂Cl₂:MeOH=88:12) to give compound 23 (2.05 g, 70%) as a white solid. mp 310-312° C.; UV (MeOH)λₘₐₓ: 272, 300 nm; ¹H-NMR (DMSO-d₆, 500 MHz) δ 12.82 (brs, 1H), 11.35 (s, 1H), 10.36 (s, 1H), 8.02 (s, 1H), 6.48 (s, 1H), 2.58 (m, 1H), 1.40 (d, J=5.5 Hz, 6H); ¹³C-NMR (CDCl₃, 125 MHz) δ 177.3, 155.2, 142.3, 140.7, 138.0, 124.5, 83.5, 35.6, 19.5; HRMS (ES) calcd for C₁₀H₁₄N₄O₂ (M−H⁺) 221.1039, found 221.1018.

### (1′S,2′R,3′R)-5-(Cyanomethyl)-3-[2′,3′-(Isopropylidenedioxy)-4′-(trityloxymethyl)-4′-cyclopenten-1′-yl]-3H-imidazole-4-carboxylic acid methyl ester (20)

To a solution of 5 (0.5 g, 1.16 mmol) in anhydrous THF (25 mL), PPh₃ (0.61 g, 2.33 mmol) and 19 (0.29 g, 1.75 mmol) was added under N₂ atmosphere at room temperature. The mixture was cooled to −78° C. DIAD (0.46 mL, 2.33 mmol) was slowly added to this mixture and stirred for over night at rt. The mixture was concentrated under reduced pressure and the residue was purified by silica gel chromatography (hexane:EtOAc=4:1) to give compound 20 (0.61 g, 91%) as a white solid. mp 52° C.; [α]²⁵_{D} 31.02 (c 1.1, CHCl₃); UV (MeOH) λₘₐₓ 207, 232; ¹H-NMR (CDCl₃, 500 MHz) δ 7.47-7.44 (m, 7H), 7.33-7.25 (m, 9H), 6.03 (s, 1H), 5.98 (s, 1H), 5.09 (d, J=5.5 Hz, 1H), 4.50 (d, J=5.5 Hz, 1H), 4.03 (dd, J=2.5, 15.5 Hz, 1H), 4.01 (d, J=2.5 Hz, 2H), 3.98 (s, 3H), 3.83 (d, J=15.5 Hz, 1H), 1.40 (s, 3H), 1.31 (s, 3H); ¹³C-NMR (CDCl₃, 125 MHz) δ 160.1, 151.1, 143.6, 140.7, 138.5, 128.5, 128.0, 127.3, 121.4, 119.6, 116.7, 112.5, 87.3, 85.2, 83.5, 67.1, 61.2, 52.2, 27.5, 26.2, 19.3; HRMS (ES) calcd for C₃₅H₃₃N₃O₅ (M+H⁺) 576.2499, found 576.2403.

### (1′S,2′S,3′R)-(2′,3′-(isopropylidenedioxy)-4′-(trityloxymethyl)-4′-cyclopenten-1′-yl)-3-deaza guanosine (21)

Compound 20 (110 mg, 0.17 mmol), liquid NH₃ were placed in a steel bomb and heated for 18 h at 100° C. The ammonia was allowed to evaporate at room temperature. The crude residue was purified on silica gel column (CH₂Cl₂:MeOH=95:5) to give the protected intermediate and which is treated with HCl (0.20 mL) in MeOH (10 mL), THF (10 mL) and water (1 mL) was heated to 50° C. for 10 h. The mixture was concentrated under reduced pressure, dissolved in water (10 mL), washed with CH₂Cl₂ (2×20 mL) and the water layer was concentrated and purified using C-18 reverse phase silica gel chromatography (water) to give 27 (33 mg, 56%) as a white solid. mp 190° C. (color change); [α]²⁴_{D} −105.57 (c 0.2, MeOH); UV λₘₐₓ 279, 318 (ε 10 920, 5650, pH 2), 261, 317 (ε 5750, 6900, pH 7), 263, 312 (ε 5620, 5100, pH 11); ¹H-NMR (DMSO, 500 MHz) δ 10.82 (s, 1H, NH), 8.32 (s, 1H), 5.94 (brs, 2H, NH₂), 5.76 (d, J=2 Hz, 1H), 5.50 (s, 1H), 5.20 (brs, 2H, OH), 5.09 (brs, 1H), 5.01 (brs, 1H, OH), 4.38 (d, J=5.5 Hz, 1H), 4.14 (m, 2H), 4.07 (t, J=6.0 Hz, 1H), 3.78 (d, J=15 Hz, 1H), 3.73 (d, J=15 Hz, 1H), 1.35 (s, 3H), 1.26 (s, 3H); ¹³C-NMR (DMSO, 125 MHz) δ 156.9, 148.2, 148.0, 128.51, 111.9, 68.9, 83.9, 70.4, 65.6, 61.5; HRMS (ES) calcd for C₁₂H₁₄N₄O₄ (M+H⁺) 279.1094, found 279.1091.

### (1S,2R,3R)-[2,3-(Isopropylidenedioxy)-4-(trityloxymethyl)-4-cyclopenten-1-yl]-isobutyryl-3-deazaguanine (24)

To a stirring solution of 23 (0.80 g, 1.57 mmol) in anhydrous DMF (20 mL), NaH (0.06 g, 2.36 mmol) and 18-crown-6 (0.50 g, 1.89 mmol) was added and stirred for 30 min at room temperature under N₂ atmosphere. Compound 22 (0.42 g, 1.89 mmol) in DMF (5 mL) was added to the mixture and stirred at 60° C. for 24 h. The DMF was removed under reduced pressure. The crude residue was neutralized with sat NH₄Cl solution and extracted with EtOAc (150 mL×2). The combined organic layer was dried with Na₂SO₄, concentrated under reduced pressure and then purified using silica gel chromatography (CH₂Cl₂:MeOH=96:4) to give compound 24 (0.4 g, 40%) as a white solid. mp 98° C.; [α]²⁶_{D} −18.80 (c 1.0, MeOH); ¹H-NMR (CDCl₃, 500 MHz) δ 11.93 (s, 1H), 10.86 (s, 1H), 7.46-7.44 (m, 7H), 7.32-7.25 (m, 10H), 6.11 (s, 1H), 5.31 (s, 1H), 5.20 (d, J=6.5 Hz, 1H), 4.62 (d, J=7.5 Hz, 1H), 3.90 (d, J=19.5 Hz, 1H), 3.87 (d, J=20 Hz, 1H), 2.85 (m, 1H), 1.36 (s, 3H), 1.25 (s, 3H), 1.17 (d, J=5.5 Hz, 3H), 1.16 (d, J=5.5 Hz, 3H); ¹³C-NMR (CDCl₃, 125 MHz) δ 180.2, 177.3, 157.3, 150.4, 143.8, 143.7, 141.0, 138.8, 138.7, 128.5, 127.9, 127.8, 127.2, 127.1, 127.0, 112.7, 87.3, 84.5, 83.8, 66.1, 61.4, 35.0, 27.3, 25.9, 19.5, 19.3; HRMS (ES) calcd for C₃₈H₃₈N₄O₅ (M+H⁺) 631.2921, found 631.2921.

### (1S,2R,3R)-[2,3-(Isopropylidenedioxy)-4-(trityloxymethyl)-4-cyclopenten-1-yl]-3-deaza guanine (26).

Compound 24 (0.4 g, 0.63 mmol) in 20 mL of methanol was saturated with NH₃ gas and the solution was quickly transferred to the steal bomb. The mixture was heated at 110° C. for 20 h. The steel bomb was cooled to room temperature, methanol was removed under reduced pressure and the residue was purified using silica gel chromatography (CH₂Cl₂:MeOH=9:1) to afford 26 (0.33 mg, 0.58 mmol) in 92% yield as a white solid. mp 175° C.; [α]²⁴_{D} +4.82 (c 1, CHCl₃); UV (MeOH) λₘₐₓ 275, 301; ¹H-NMR (DMSO, 500 MHz) δ 10.34 (s, 1H), 7.52 (s, 1H), 7.45-7.28 (m, 15H), 6.15 (s, 1H), 5.61 (s, 2H), 5.36 (s, 1H), 5.26 (d, J=5.5 Hz, 1H), 5.24 (s, 1H), 4.56 (d, J=5.5 Hz, 1H), 3.78 (d, J=15 Hz, 1H), 3.73 (d, J=15 Hz, 1H), 1.35 (s, 3H), 1.26 (s, 3H); ¹³C-NMR (DMSO, 100 MHz) δ 156.9, 148.2, 148.0, 144.0, 142.6, 137.3, 128.56, 128.51, 127.6, 123.1, 111.9, 68.9, 84.3, 83.9, 70.4, 65.6, 61.5, 27.5, 26.0; HRMS (ES) calcd for C₃₈H₃₈N₄O₅ (M+H⁺) 561.2503, found 561.2609.

### (1S,2R,3R)-[2,3-dihydroxy-4-(hydroxymethyl)-4-cyclopenten-1-yl]-3-deaza guanine (27).

A mixture of compound 26 (0.20 g) and HCl (0.20 mL) in MeOH (10 mL), THF (10 mL) and water (1.0 mL) was heated to 50° C. for 10 h. The mixture was concentrated under reduced pressure. The residue was dissolved in 10 mL of water and the resulting solution was washed with CH₂Cl₂ (20 mL×2). The remaining aqueous layer was concentrated and purified using C-18 reverse phase silica gel chromatography (acetonitrile:water=8:2) to give 27 (0.07 mg, 64%) as pure white solid. mp 198° C. (color change); [α]²⁴_{D} −105.57 (c 0.2, MeOH); UV λₘₐₓ 283, 312 (ε 13000, 6700, pH 2), 271, 300 (ε 11700, 8540, pH 7), 272, 295 (ε 12 500, pH 11); ¹H-NMR (DMSO, 500 MHz) δ 10.82 (s, 1H, NH), 8.32 (s, 1H), 5.94 (brs, 2H, NH₂), 5.76 (d, J=2 Hz, 1H), 5.50 (s, 1H), 5.20 (brs, 2H, OH), 5.09 (brs, 1H), 5.01 (brs, 1H, OH), 4.38 (d, J=5.5 Hz, 1H), 4.14 (m, 2H), 4.07 (t, J=6.0 Hz, 1H), 3.78 (d, J=15 Hz, 1H), 3.73 (d, J=15 Hz, 1H), 1.35 (s, 3H), 1.26 (s, 3H); ¹³C-NMR (DMSO, 125 MHz) δ 156.9, 148.2, 148.0, 128.51, 111.9, 68.9, 83.9, 70.4, 65.6, 61.5; HRMS (ES) calcd for C₁₂H₁₄N₄O₄ (M+H⁺) 279.1094, found 279.1091; Anal. calcd for C₁₂H₁₄N₄O₄HCl: C, 45.80, H, 4.80, N, 17.80. found C, 45.76; H, 4.83; N, 17.72.

### References for Novel Cyclpentenoside Compounds (5-Membered Rings)

1. (a) Yaginuma, A.; Muto, N.; Tsujino, M.; Sudate, Y.; Hayashi, M.; Otani, M. I. J. Antibiot. 1981, 34, 359-366. (b) Hayashi, M.; Yaginuma, A.; Nakatsu, K. J. Antibiot. 1981, 34, 675-680.
2. (a) De Clercq, E. Antimicrob. Agents Chemother. 1985, 28, 84-89. (b) Marquez, V. E.; Lim, M. I.; Med. Res. Rev. 1986, 6, 1-40. (c) Agrofoglio, L.; Suhas, E.; Farese, A.; Condom, R.; Challand, S. R.; Earl, R. A.; Guedj, R. Tetrahedron 1994, 50, 10611-10670.
3. (a) Ueland, P. M. Pharmacol. Rev. 1982, 34, 223-253. (b) Borchardt, R. T.; Keller, B. T.; Patel-Thombre, U. J. Biol. Chem. 1984, 259, 4353-4358. (c) De Clercq, E.; Cools, M. Biochem. Biophys. Res. Commun. 1985, 129, 306-311. (d) Wolfe, M. S.; Borchardt, R. T. J. Med. Chem. 1991, 34,1521-1530. (e) Turner, M. A.; Yang, X.; Yin, D.; Kuczera, K.; Borchardt, R. T.; Howell, P. L. Cell Biochem. Biophys. 2000, 33, 101-125.
4. (a) Glazer, R. I.; Knode, M. C. J. Biol. Chem. 1984, 259, 12964-12969. (b) Hoshi, A.; Yoshida, M.; Iigo, M.; Tokuzen, R.; Fukukawa, K.; Ueda, T.J Plarmacobio-Dyn. 1986, 9, 202-206. (c) Inaba, M.; Nagashima, S.; Tsukagoshi, S.; Sakurai, Y. Cancer Res. 1986, 46, 1063-1067.
5. (a) Borcherding, D. R.; Scholtz, S. A.; Borchart, R. T. J. Org. Chem. 1987, 52, 5457. (b) Hegedus, L. S.; Geisler, L. J Org, Chem. 2000, 65, 4200-4203. (c) Wolfe, M. S.; Lee, Y.; Bartlett, W. J.; Borcherding, D. R.; Borchardt, R T. J. Med. Chem. 1992, 35, 1782-1791 and references cited therein. (d) Tseng, C. K. H.; Marquez, V. E.; Fuller, R. W.; Goldstein, B. M.; Haines, D. P; McPherson, H.; Parsons, J. L.; Shannon, W. M.; Arnett, G.; Hollingshead, M.; Driscoll, J. S. J. Med. Chem. 1989, 32, 1442-1446. (e) Shuto, S.; Minakawa, N.; Niizunia, S.; Kin, H-S.; Wataya, Y.; Matsuda, A. J. Med. Chem. 2002, 45, 748-751 and references cited therein. (f) Moon, H. R.; Kim, H. O.; Lee, K. M.; Chun, M. W.; Kim, J. H.; Jeong, L. S. Org. Lett. 2002, 4, 3501-3503. (g) Seley, K. L.; Mosley, S. L.; Zeng, F. Org. Lett. 2003, 5, 4401-4403. (h) Jeong, L. S.; Yoo, S. J.; Lee, K. M.; Koo, M. J.; Choi, W. J.; Kim, H. O.; Moon, H. R.; Lee, M. Y.; Park, J. G.; Lee, S. K; Chun, M. W. J. Med. Chem. 2003, 46, 201-203. (i) Shuto, S.; Obara, T.; Saito, Y.; Andrei, G.; Snoeck, R.; De Clercq, E.; Matsuda, A.J. Med. Chem. 1996, 39, 2392-2399. (j) Wang, P.; Gullen, B.; Newton, M. G.; Cheng, Y—C.; Schinazi, R. F.; Chu, C. K J. Med. Chem. 1999, 42, 3390-3399. (k) Borcherding, D. R.; Narayanan, S.; Hasobe, M.; McKee, J. G.; Keller, B. T.; Borchardt, R T. J. Med. Chem. 1988, 31, 1729-1738.
6. (a) Song, G. Y.; Paul, V.; Choo, H.; Morrey, J.; Sidwell, R. W.; Schinazi, R. F.; Chu, C. K. J. Med. Chem. 2001, 44, 3985-3993. (b) Jin, Y. H.; Liu, P.; Wang, J.; Baker, R.; Huggins, J.; Chu, C. K. J. Org. Chem. 2003, 68, 9012-9018. (c) Chu, C. K; Jin, Y. H.; Baker, R. O.; Huggins, J. Bioorg. Med. Chem. Lett. 2003, 13, 9-12.
7. (a) Arita, M.; Adachi, K; Ito, Y.; Sawai, H.; Ohno, M. Enantioselective J. Am. Chem. Soc. 1983, 105, 4049-4055. (b) Hill, J. M.; Hutchinson, E. J.; Le Grand, D. M.; Roberts, S. M. J. Chem. Soc., Perkin Trans. 1 1994, 1483-1487. (c) Trost, B. M.; Madsen, R; Guile, S. D.; Brown, B. J. Am. Chem. Soc. 2000, 122, 5947-5956. (d) Marquez, V. E.; Limn, M-I.; Tseng, C. K.-H.; Markovac, A.; Priest, M. A.; Khan, M. S.; Kaskar, B.J. Org. Chem. 1988, 53, 5709-5714. (e) Bestmann, H. J.; Roth, D. Angew. Chem. Int. Ed. Engl. 1990, 29, 99-100. (f) Ono, M.; Nishimura, K.; Tsubouchi, H.; Nagaoka, Y.; Tomioka, K J Org. Chem. 2001, 66, 8199-8203.
8. (a) Grubbs, R. H.; Miller, S. J.; Fu, G. C. Acc. Chem. Res. 1995, 28, 446-452. (b) Schuster, M.; Blechert, S.Angew. Chem. Int. Ed. Engl. 1997, 36, 2037-2056. (c) Amstrong, S. K.J. Chem. Soc., Perkin trans. 1 1998, 371-388. (d) Grubbs, P, H.; Chang, S. Tetrahedron 1998, 54, 4413-4450. (e) Trnka, T. M.; Grubbs, R. H.Acc. Chem. Res. 2001, 34, 18-29. (f) Deiters, A.; Martin, S. F. Chem. Rev. 2004, 104, 2199-2238 and references cited therein.
9. (a) Crimmins, M. T.; King, B. W.; Zuercher, W. J.; Choy, A. J. Org. Chem. 2000, 65, 8499-8509. (b) Choi, W. J.; Park, J. G.; Yoo, S. J.; Kim, H. O.; Moon, H. R.; Chun, M. W.; Jung, Y. H.; Jeong, L. S. J. Org. Chem. 2001, 66, 6490-6494. (c) Gurjar, M. K.; Maheshwar, K. J. Org. Chem. 2001, 66, 7552-7554. (d) Jin, Y. H.; Chu, C. K. Tetrahedron Lett. 2002, 43, 4141-4143. (e) Choi, W. J.; Moon, H. R.; Kim, H. O.; Yoo, B. Y.; Lee, J. A.; Shin, D. H.; Jeong, L. S. J. Org. Chem. 2004, 69, 2634-2636. (f) Yang, M.; Ye, W.; Schneller, S. W. J. Org. Chem. 2004, 69, 3993-3996. (g) Davis, F. A.; Wu, Y. Org. Lett. 2004, 6, 1269-1272.
10. (a) Lee, K; Cass, C.; Jacobson, K. A. Org. Lett. 2001, 3, 597-599. (b) Callam, C. S.; Lowary, T. L. J. Org. Chem. 2001, 66, 8961-8972. (c) Kim, A.; Hong, J. H. Nucleosides Nucleotides & Nucleic Acids 2004, 23, 813-822.
11. (a) Seepersaud, M; Al-Abed, Y. Org. Lett. 1999, 1, 1463-1465. (b) Seepersaud, M.; Al-Abed, Y.Tetrahedron Lett. 2000, 41, 7801-7803. (c) Gillaizeau, I.; Charamon, S.; Agrofoglio, L. A. Tetrahedron Lett. 2001, 42, 8817-8819.
12. Niizuma, S.; Shuto, S.; Matsuda, A. Tetrahedron 1997, 53, 13621-13632.
13. (a) Scholl, M.; Lee, C. W.; Grubbs, R, H.Org. Lett. 1999, 1, 953-956. (b) Chatterjee, A. K.; Grubbs, R. H. Org. Lett. 1999, 1, 1751-1753. (c) Kirkland, T. A.; Grubbs, R. H. J. Org. Chem. 1997, 62, 7310-7318.
14. Ohira, S.; Sawamoto, T.; Yamato, M. Tetrahedron Lett. 1995, 36, 1537-1538: [α]²⁵_{D} +28.4 (c 1.00, CHCl₃); Compound (+)-12a: [α]²³_{D} +33.2 8774 (c 1.00, CHCl₃).
15. Gathergood, N.; Knudsen, K R.; Jorgensen, K. A. J. Org. Chem. 2001, 66, 1014-1017.
16. (a) Mitsunobu, O. Synthesis 1981, 1-26. (b) Hassner, A.; Dehaen, W. J. Org. Chem. 1990, 55, 2243-2245. (c) Ahn, C.; Correia, R.; Desong, P. J. Org. Chem. 2002, 67, 1751-1753.

17. Wang, Q.; Chan, T. R.; Hilgraf, R.; Fokin, V. V.; Sharpless, K. B.; Finn, M. G. Bioconjucation by Copper(I)-Catalyzed Azide-Alkyne [3+2] Cycloaddition. J. Am. Chem. Soc. 2003, 125, 3192-3193 and references cited therein.

### References for 3-Deazaadenine Synthesis and (−)-3-Deazaneplanocin A

1. (a) Borchardt, R. T. In The Biochemistry of Adenosylmethionine; Salvatore, F., et al., Eds.; Columbia University Press: New York, 1977; pp 151-171.; (b) Ueland, P. M. Pharmacol. Rev. 1982, 34, 223-253. (c) Wolfe, M. S.; Borchardt, R. T. J. Med. Chem. 1991, 34, 1521-1530. (d) Turner, M. A.; Yang, X.; Yin, D.; Kuczera, K.; Borchardt, R. T.; Howell, P. L. Cell Biochem. Biophys. 2000, 33, 101-125.
2. (a) De Clercq, E.; Cools, M. Biochem. Biophys. Res. Commun. 1985, 129, 306-311. (b) Cantoni, G. L. In Biological Methylation and Drug Design; Borchardt, R. T., Creveling, C. R., Ueland, P. M., Eds.; Humana Press: Clifton, N.J., 1986; pp 227-238.; (c) Borcherding, D. R.; Scholtz, S. A.; Borchardt, R. T. J. Org. Chem. 1987, 52, 5457-5461.; (d) Siddiqi, S. M.; Chen, X.; Rao, J.; Schneller, S. W. J. Med. Chem. 1995, 38, 1035-1038. (e) Jin, Y. H.; Liu, P.; Wang, J.; Baker, R.; Huggins, J.; Chu, C. K. J. Org. Chem. 2003, 68, 9012-9018.
3. (a) Tseng, C. K. H.; Marquez, V. E.; Fuller, R. W.; Goldstein, B. M.; Haines, D. R.; McPherson, H.; Parsons, J. L.; Shannon, W. M.; Arnett, G.; Hollingshead, M.; Driscoll, J. S. J. Med. Chem. 1989, 32, 1442-1446. (b) Glazer, R. I.; Knode, M. C.; Tseng, C. K. H.; Haines, D. R.; Marquez, V. E. Biochem. Pharmacol. 1986, 35, 4523-4527.
4. (a) Shuto, S.; Minakawa, N.; Niizuma, S.; Kim, H-S.; Wataya, Y.; Matsuda, A. J. Med. Chem. 2002, 45, 748-751. (b) Gerster, J. F.; Lindstrom, K J.; Miller, R. L.; Tomai, M. A.; Birmachu, W.; Bomersine, S. N.; Gibson, S. J.; Imbertson, L. M.; Jacobson, J. R.; Knafla, R. T.; Maye, P. V.; Nikolaides, N.; Oneyemi, F. Y.; Parkhurst, G. J.; Pecore, S. E.; Reiter, M. J.; Scribner, L. S.; Testerman, T. L.; Thompson, N. J.; Wagner, T. L.; Weeks, C. E.; Andre, J-D.; Lagain, D.; Bastard, Y.; Lupu, M. J. Med. Chem. 2005, 48, 3481-3491. (c) Seley, K. L.; O'Daniel, P. I.; Salim, S. Nucleosides Nucleotides & Nucleic acids 2003, 22, 2133-2144. (d) Obara, T.; Shuto, S.; Saito, Y.; Snoeck, R.; Andrei, G.; Balzarini, J.; De Clercq, E.; Matsuda, A. J. Med. Chem. 1996, 39, 3847-3852. (e) Lee, J. A.; Moon, H. P; Kim, H. O.; Kim, K. R; Lee, K. M.; Kim, B. T.; Hwang, K. J.; Chun M. W.; Jacobson, K A.; Jeong, L. S. J. Org. Chem. 2005, 70, 5006-5013. (f) Liu, S.; Yuan, C-S.; Borchardt, R. T. J. Med. Chem. 1996, 39, 2347-2352. (g) Montgomery, J. A.; Clayton, S. J. J. Med. Chem. 1982, 25, 96-98. (h) Franchetti, P.; Cappellacci, L.; Grifantini, M.; Messini, L.; Sheikha, G. A.; Loi, A. G.; Tramontano, E.; De Montis, A.; Spiga, M. G.; Colla, P. J.Med. Chem. 1994, 37, 3534-3541. (i) Antonini, I.; Cristalli, G.; Franchetti, P.; Grifantini, M.; Martelli, S.; Lupidi, G.; Riva, F. J. Med. Chem. 1984, 27, 274-278. (j) Houston, D. M.; Dolence, E. K; Keller, B. T.; Patel-Thombre, U.; Borchardt, R. T. J. Med. Chem. 1985, 28, 467-471.) Minakawa, N.; Kojima, N.; Matuda, A. J. Org. Chem. 1999, 64, 7158-7172.
5. (a) Montgomery, J. A.; Clayton, S. J.; Thomas, H. J.; Shannon, W. M.; Arneft, G.; Bodner, A. J.; Kion, I-K.; Cantoni, G. L.; Chiang, P. K J. Med. Chem. 1982, 25, 626-629. (b) Houston, D. M.; Dolence, E. K.; Keller, B. T.; Patel-Thombre, U.; Borchardt, R. T. J. Med. Chem. 1985, 28, 471-477.
6. (a) Rousseau, R. J.; Townsend, L. B.; Robins, R. Biochemistry 1966, 5, 756-760. (b) Seela, F.; Rosemeyer, H.; Fischer, S. Helv. Chem. Acta 1990, 73, 1602-1611. (c) Serafinowski, P. Synthesis 1990, 757-760. (d) Volpini, R.; Camaioni, E.; Costanzi, S.; Vittori, S.; Cristaili, G. Helv. Chem. Acta 1998, 81, 2326-2331. (e) Minakawa, N.; Matsuda, A. Tetrahedron Lett. 1993, 34, 661-664.
7. Yang, M.; Zhou, J.; Schneller, S. W. Tetrahedron Lett. 2004, 45, 8981-8982.
8. Rousseau, R. J.; Robins, R. K. J Heterocyclic Chem. 1965, 2, 196-201.
9. Cho, J. H.; Bernard, D. L.; Sidwell, R W.; Kern, E. R.; Chu, C. K. J. Med. Chem. 2005, in progress.
10. Song, G. Y.; Paul, V.; Choo, H.; Morrey, J.; Sidwell, R. W.; Schinazi, R. F.; Chu, C. K. J. Med. Chem. 2001, 44, 3985-3993.
11. (a) Davis, J. H.; Fox. P. A. Chem. Commun. 2003, 1209-1212. (b) Sheldon, R. Chem. Commun. 2001, 2399-2407. (c) Wasserscheid, P.; Kein, W. Angew. Chem. Int. Ed. 2000, 39, 3772-3798. (d) Welton, T. Chem. Rev. 1999, 99, 2071-2083.
12. Recycling reaction: A solution of 6-chloro-3-deazapurine 8 (0.25 g, 1.54 mol) in 0.6 mL of DMF-[emim]BF4 (10:1 v/v) was treated with 2.5 equiv. of LiN₃ (0.19 g, 3.85 mmol, 20 wt % in water) at 80° C. for 6 h, and then the ionic liquid solution was recovered. To the recovered ionic liquid solution, compound 8 (0.25 g, 1.54 mmol), 1.1 equiv. of LiN₃ (0.4 mL), water (0.6 mL) and DMF (0.5 mL) were added at room temperature, and then the reaction was repeated under the same reaction time. The compound 9 was obtained in 94-99% yields.
13. Bullock M. W.; Hand, J. J.; Stokstad, E. L. R. J. Org. Chem. 1957, 22, 568-569.
14. (a) Dey, S.; Garner, P. J. Org. Chem. 2000, 65, 7697-7699. (b) Grehn, L.; Ragnarsson, U. Angew. Chem. Int. Ed. 1984, 23, 296-301.
15. Milecki, J.; Foldesi, A.; Fischer, A.; Adamiak, R. A.; Chattopadhyaya, J. J Labelled Cpd. Radiopharm. 2001, 44, 763-783.
16. Hasan, I.; Marinelli, E. R.; Lin, L-C. C.; Fowler, F. W.; Levy, A. B. J. Org. Chem. 1981, 46, 175-164.

### References for Synthesis and Antiviral Activity of 7-Deaza Neplanocin A Against Orthopox Viruses (Vaccinia and Cowpox Virus)

1. Yaginuma, A.; Muti, N.; Tsujinio, M.; Sudate, Y.; Hayashi, M.; Otani, M. J. Antibiot. 1981, 34,359.110
2. De Clercq, E. Antimicrob. Agents Chemother. 1985, 28, 84.
3. Borchardt, R. T.; Keller, B. T.; Patel-Thombre, U. J. Biol. Chem. 1984, 259, 4353.
4. Shuto, S.; Minakawa, N.; Niizuma, S.; Kim, H.-S.; Wataya, Y.; Matsuda, A. J. Med. Chem. 2002, 45, 7480.
5. Yang, M.; Schneller, S. W.; Korba, B. J. Med. Chem. 2005, 48, 5043.
6. Obara, T.; Shuto, S.; Saito, Y.; Snoeck, R.; Andrei, G.; Balzarini, J.; De Clercq, E.; Matsuda, A. J. Med. Chem. 1996, 39, 3847.120
7. Tseng, C. K H.; Marquez, V. E.; Fuller, R. W.; Goldstein, B. M.; Arnett, G.; Hollingshead, M.; Driscoll, J. S. J. Med. Chem. 1989, 32, 1442.
8. Eldrup, A. B.; Prhavc, M.; Brooks, J.; Bhat, B.; Prakash, T. P.; Song, Q.; Bera, S.; Bhat, N.; Dande, P.; Cook, P. D.; Bennett, C. F.; Carroll, S. S.; Ball, R. G.; Bosserman, M.; Burlein, C.; Colwell, L. F.; Fay, J. F.; Flores, O. A.; Getty, K.; LaFemina, R. L.; Leone, J.; MacCoss, M.; McMasters, D. R.; Tomassini, J. E.; Langen, D. V.; Wolanski, B.; Olsen, D. B. J. Med. Chem. 2004, 47, 5284.130
9. Fenner, F. In Poxviruses in Virology 2nd Ed, Fields, Ed, Raven Press: New York; Vol. 2, Chapter 75.
10. De Clercq, E.; Cools, M.; Balzarini, J.; Marquez, V. E.; Borcherding, D. R.; Borchardt, R. T.; Drach, J. C.; Kitaoka, S.; Konno, T. Antimicrob. Agents Chemother. 1989, 33, 1291.
11. (a) Chu, C. K.; Jin, Y. H.; Baker, R. O.; Huggins, J. Bioorg. Med. Chem. Lett. 2003, 13, 9; (b) Yang, M.; Schneller, S. W. Bioorg. Med. Chem. Lett. 2005, 15, 149; (c) Roy, A.; Schneller, S. W.; Keith, K. A.; Hartline, C. B.; 140 Kern, E. R. Bioorg. Med. Chem. 2005, 13, 4443.
12. Song, G. Y.; Paul, V.; Choo, H.; Morrey, J.; Sidwell, R. W.; Schinazi, R. F.; Chu, C. K. J. Med. Chem. 2001, 44, 3985.
13. 6-Chloro-7-deazapurine is commercially available at Toronto Research Chemicals, Inc.
14. Data for compound 2: mp 196-198_C; ½a_{—}23 D_{—}43.80 (c 0.10, MeOH); ¹H NMR (500 Hz, DMSO-d6) d 8.04 (s, 1H), 7.00 (d, 3.5 Hz, 1H), 6.94 (br s, 2H), 6.54 (d, 3.5 Hz, 1H), 5.58 (m, 1H), 5.52 (m, 1H), 4.99 (br s, 1H), 4.88 (br s, 150

### References for Synthesis of 3-Deazaneplanocin A and 3-Deazaguanosine-3′-deoxy carbocyclic cyclopentenyl nucleosides

(1) (a) Krenitsky, T. A.; Rideout, J. L.; Chao, E. Y.; Koszalka, G. W.; Gurney, F.; Crouch, R. C.; Cohn, N. K.; Wolberg, G.; Vinegar, R. Imidazo[4,5-c]pyridines (3-Deazapurines) and Their Nucleosides as Immunosuppressive and Antiinflammatory Agents. J. Med. Chem. 1986, 29, 1035-1038. (b) Rousseeau, R. J.; Townsend, L. B.; Robins, R. K. The Synthesis of 4-Amino-β-D-ribofuranosylimidazo[4,5-c]pyrimidine (3-Deazaadenosine) and Related Nucleosides. Biochemistry 1966, 5, 756-760.
(2) (a) Tseng, C. K. H.; Marquez, V. E.; Fuller, R. W.; Goldstein, B. M.; Haines, D. R.; McPherson, H.; Parsons, J. L.; Shannon, W. M.; Arnett, G.; Hollingshead, M.; Driscoll, J. S. Synthesis of 3-Deazaneplanocin A, a Powerful Inhibitor of S-Adenosylhomocysteine Hydrolase with Potent and Selective in Vitro and in Vivo Antiviral Activities.J. Med. Chem. 1989, 32, 1442-1446. (b) Glazer, R. I.; Inode, M. C.; Tseng, C. K. H.; Haines, D. R.; Marquez, V. E. 3-Deazaneplanocin A: a New Inhibitor of S-Adenosylhomocysteine Hydrolase Synthesis and Its Effects in Human Colon Carcinoma Cells. Biochem. Pharmacol. 1986, 35, 4523-4527.
(3) (a) Borchardt, R. T. In The Biochemistry of Adenosylmethionine; Salvatore, F., et al., Eds.; Columbia University Press: New York, 1977; pp 151-171.; (b) Ueland, P. M. Pharmacological and Biochemical Aspects of S-Adenosylhomocysteine and S-Adenosylhomocysteine Hydrolase. Pharmacol. Rev. 1982, 34, 223-253. (c) Wolfe, M. S.; Borchardt, R. T. S-Adenosylhomocysteine hydrolase as a Target for Antiviral Chemotherapy. J. Med. Chem. 1991, 34, 1521-1530. (d) Turner, M. A.; Yang, X.; Yin, D.; Kuczera, K; Borchardt, R. T.; Howell, P. L. Structure and Function of S-Adenosylhomocysteine hydrolase. Cell Biochem. Biophys. 2000, 33, 101-125. (d) Guranowski, A.; Montgomery, J. M.; Cantoni, G. L.; Chiang, P. K. Adenosine Analogues as Substrates and Inhibitors of S-Adenosylhomocysteine Hydrolase.

### Biochemistry 1981, 20, 110-1115.

(4) (a) De Clercq, E.; Cools, M. Antiviral Potency of Adenosine Analogues: Correlation with Inhibition of S-Adenosylhomocysteine Hydrolase. Biochem. Biophys. Res. Commun. 1985, 129, 306-311. (b) Cantoni, G. L. In Biological Methylation and Drug Design; Borchardt, R. T., Creveling, C. R., Ueland, P. M., Eds.; Humana Press: Clifton, N.J., 1986; pp 227-238. (c) Borcherding, D. R; Scholtz, S. A.; Borchardt, R. T. Synthesis of Analogues of Neplanocin A: Utilization of Optically Active Dihydroxycyclopentanones Derived from Carbohydrate. J. Org. Chem. 1987, 52, 5457-5461. (d) Siddiqi, S. M.; Chen, X.; Rao, J.; Schneller, S. W. 3-Deaza and 7-Deaza-5′-noraristeromycin and Their Antiviral Properties. J. Med. Chem. 1995, 38, 1035-1038. (e) Jin, Y. H.; Liu, P.; Wang, J.; Baker, R.; Huggins, J.; Chu, C. K. Practical Synthesis of D- and L-2-Cyclopentanone and Their Utility for the Synthesis of Carbocyclic Antiviral Nucleosides against Orthopox Viruses (Smallpox, Monkeypox, and Cowpox Virus). J. Org. Chem. 2003, 68, 9012-9018.
(5) (a) Revankar, G. R.; Gupta, P. K.; Adams, A. D.; Dalley, N. K.; McKernan, P. A.; Cook, P. D.; Canonico, P. G.; Robins, R. K. Synthesis and Antiviral/Antitumor Activities of Certain 3-Deazaguanine Nucleosides and Nucleotides. J. Med. Chem. 1984, 27, 1389-1396. (b) Avila, J. L.; Rojas, T.; Avila, A.; Polegre, M. A.; Robins, R. K. Biological Activity of Analogs of Guanine and Guanosine Against American Trypanosoma and Leishmania spp. Antimicrobial Agents And Chemotherapy 1987, 447-451. (c) Sidwell, R. W.; Huffman, J. H.; Barnard, D. L.; Smee, D. F.; Warren, R. P.; Chirigos, M. A.; Kende, M.; Huggins, J. Antiviral and Immunomodulating Inhibitors of Experimentally-Induced Punta Toro Virus Infections. Antiviral Research 1994, 25, 105-122.
(6) (a) Shuto, S.; Minakawa, N.; Niizuma, S.; Kim, H-S.; Wataya, Y.; Matsuda, A. New Neplanocin Analogues. 12. Alternative Synthesis and Antimalarial Effect of (6′R)-6′-C-Methylneplanocin A, a Potent AdoHcy Hydrolase Inhibitor. J. Med. Chem. 2002, 45, 748-751. (b) Gerster, J. F.; Lindstrom, K J.; Miller, R. L.; Tomai, M. A.; Birmachu, W.; Bomersine, S, N.; Gibson, S. J.; Imbertson, L. M.; Jacobson, J. R.; Knafla, R T.; Maye, P. V.; Nikolaides, N.; Oneyemi, F. Y.; Parkhurst, G. J.; Pecore, S. E.; Reiter, M. J.; Scribner, L. S.; Testerman, T. L.; Thompson, N.J.; Wagner, T. L.; Weeks, C. E.; Andre, J-D.; Lagain, D.; Bastard, Y.; Lupu, M. Synthesis and Structure-Activity-Relationships of 1H-Imidazo[4,5-c]quinolines That Induce Interferon Production. J. Med. Chem. 2005, 48, 3481-3491. (c) Seley, K. L.; O'Daniel, P. I.; Salim, S. Design and Synthesis of a Series of Chlorinated 3-Deazaadenine Analogues.Nucleosides Nucleotides & Nucleic acids 2003, 22, 2133-2144. (d) Obara, T.; Shuto, S.; Saito, Y.; Snoeck, R.; Andrei, G.; Balzarini, J.; De Clercq, E.; Matsuda, A. New Neplanocin Analogues. 7. Synthesis and Antiviral Activity of 2-Halo Derivatives of Neplanocin A. J. Med. Chem. 1996, 39, 3847-3852. (e) Lee, J. A.; Moon, H. R.; Kiln, H. O.; Kim, K. R.; Lee, K. M.; Kim, B. T.; Hwang, K. J.; Chun M. W.; Jacobson, K. A.; Jeong, L. S. Synthesis of Novel Apio Carbocyclic Nucleoside Analogues as Selective A₃ Adenosine Receptor Agonists. J. Org. Chem. 2005, 70, 5006-5013. (f) Liu, S.; Yuan, C-S.; Borchardt, R. T. Aristeromycin-5′-carboxaldehyde: A Potent Inhibitor of S-Adenosyl-L-homocysteine Hydrolase. J. Med. Chem. 1996,39, 2347-2352. (g) Montgomery, J. A.; Clayton, S. J. 1-β-D-Arabinofuranosyl-1H-imidazo-[4,5-c]pyridine (ara-3-Deazaadenine). J. Med. Chem. 1982, 25, 96-98. (h) Franchetti, P.; Cappellacci, L.; Grifantini, M.; Messini, L.; Sheikha, G. A.; Loi, A. G.; Tramontano, E.; De Montis, A.; Spiga, M. G.; Colla, P. Synthesis and Evaluation of the Anti-HIV Activity of Aza and Deaza Analogues of IsoddA and Their Phosphates as prodrugs. J. Med. Chem. 1994, 37, 3534-3541. (i) Antonini, I.; Cristalli, G.; Franchetti, P.; Grifantini, M.; Martelli, S.; Lupidi, G.; Riva, F. Adenosine Deaminase Inhibitors. Synthesis of Deaza Analogues of erythro-9-(2-Hydroxy-3-nonyl)adenine. J. Med. Chem. 1984, 27, 274-278. (j) Houston, D. M.; Dolence, E. K.; Keller, B. T.; Patel-Thombre, U.; Borchardt, R. T. Potential Inhibitors of S-Adenosylmethionine-Dependent Methyltransferases. 8. Molecular Dissections of Carbocyclic 3-Deazaadenosine as Inhibitors of S-Adenosylhomocysteine Hydrolase. J. Med. Chem. 1985,28, 467-471. (k) Minakawa, N.; Kojima, N.; Matuda, A. Necleosides and Neceotides. 184. Synthesis and Conformational Investigation of Anti-fixed 3-Deaza-3-halopurine Ribonucleosides. J. Org. Chem. 1999, 64, 7158-7172.
(7) (a) Seela, F.; Rosemeyer, H.; Fischer, S. Synthesis of New 3′-Deoxyribonucleosides Employing the Acid-catalyzed Fusion Method. Helv. Chem. Acta 1990, 73, 1602-1611. (b) Serafinowski, P. Synthesis of 2′,3′-Dideoxy-3-deazaadenosine and Some of Its Analogues. Synthesis 1990, 757-760. (c) Volpini, R.; Camaioni, E.; Costanzi, S.; Vittori, S.; Cristalli, G. 143. Synthesis of 3-Deaza-2′-deoxyadenosine and 3-deaza-2′,3′-dideoxyadenosine; Glycosylation of the 4-Chloroimidazo[4,5-c]pyridinyl Anion. Helv. Chem. Acta 1998, 81, 2326-2331. (d) Minakawa, N.; Matsuda, A. Nucleotides and Nucleotides. 114. A Convenient Method for the Synthesis of 3-Deazapurine Nucleosides from AICA-Riboside. Tetrahedron Lett. 1993, 34, 661-664.
(8) (a) Montgomery, J. A.; Clayton, S. J.; Thomas, H. J.; Shannon, W. M.; Arnett, G.; Bodner, A. J.; Kion, I-K.; Cantoni, G. L.; Chiang, P. K. Carbocyclic Analogues of 3-Deazaadenosine: A Novel Antiviral Agent Using S-Adenosylhomocysteine Hydrolase as a Pharmacological Target. J. Med. Chem. 1982, 25, 626-629. (b) Houston, D. M.; Dolence, E. K.; Keller, B. T.; Patel-Thombre, U.; Borchardt, R. T. Potential Inhibitors of S-Adenosylmethionine-Dependent Methyltransferases. 9. 2′,3′-Dialdehyde Derivatives of Carbocyclic Purine Nucleosiddes as Inhibitors of S-Adenosylhomocysteine Hydrolase. J. Med. Chem. 1985, 28, 471-477.
(9) (a) Yang, M.; Zhou, J.; Schneller, S. W. An Efficient Synthesis of (−)-3-Deazaaristeromycin. Tetrahedron Lett. 2004, 45, 8981-8982. (b) Yang, M.; Zhou, J.; Schneller, S. W. The Mitsunobu Reaction in Preparing 3-Deazapurine Carbocyclic Nucleosides. Tetrahedron 2005, 62, 1295-1300.
(10) Cho, J. H.; Bernard, D. L.; Sidwell, R. W.; Kern, E. R.; Chu, C. K. Synthesis of Cyclopentenyl Carbocyclic Nucleosiddes as Potential Antiviral Agents Against Orthopox Viruses and SARS. J. Med. Chem. 2006, 49, 1140-1148.
(11) (a) Rousseau, R. J.; Robins, R K. The synthesis of Various Chloroimidazo[4,5-c]pyridines and related Derivatives. J Heterocyclic Chem. 1965, 2, 196-201. (b) Cosstick, R.; Li, X.; Tuli, D. K.; Williams, D. M.; Connolly, B. A.; Newman, P. C. Molecular Recognition in the Minor Groove of the DNA Helix. Studies on the Synthesis of Oligonucleotides and Polynucleotides Containing 3-Deaza-2′-deoxyadenosine. Interaction of the Oligonucleotides with the Restriction Endonuclease EcoRV. Nucleic Acids Res. 1990, 18, 4771-4777.
(12) Song, G. Y.; Paul, V.; Choo, H.; Morrey, J.; Sidwell, R W.; Schinazi, R. F.; Chu, C. K. Enantiomeric Synthesis of D- and L-Cyclopentenyl Nucleosides and Their Antiviral Activity Against HIV and West Nile Virus. J. Med. Chem. 2001, 44, 3985-3993.
(13) (a) Davis, J. H.; Fox. P. A. From Curiosities to Commodities: Ionic Liquids Begin the Transition.Chem. Commun. 2003, 1209-1212. (b) Sheldon, R Catalytic Reaction in Ionic Liquid. Chem. Commun. 2001, 2399-2407. (c) Wasserscheid, P.; Kein, W. Ionic Liquids-New “Solutions” for Transition Metal Catalysis. Angew. Chem. Int. Ed. 2000, 39, 3772-3798. (d) Welton, T. Room-temteratur Ionic Liquids. Solvents for Synthesis and Catalysis. Chem. Rev. 1999, 99, 2071-2083. (e) Dupont, J.; de Souza, R. F.; Suarez, P. A.
   Ionic Liquid (Molten Salt) Phase Organometallic Catalysis. Chem. Rev. 2002, 102, 3667-3692.
(14) Recycling procedure: A solution of 6-chloro-imidazo[4,5-c]pyridine (6) (0.25 g, 1.54 mmol) in 0.6 mL of DMF-[emim]BF4 (10:1 v/v) was treated with 2.5 equiv. of LiN₃ (0.19 g, 3.85 mmol, 20 wt % in water) at 80° C. for 6 h, and then the ionic liquid solution was recovered. To the recovered ionic liquid solution, compound 6 (0.25 g, 1.54 mmol), 1.1 equiv. of LiN₃ (0.4 mL), water (0.6 mL) and DMF (0.5 mL) were added at room temperature, and then the reaction was repeated under the same reaction time. The compound 11 was obtained in 94-99% yields.
(15) (a) Grehn, L.; Ragnarsson, U. A Convenient Method for the Preparation of 1-(tert-Butyloxycarbonyl)pyrroles. Angew. Chem. Int. Ed. 1984, 23, 296-301. (b) Dey, S.; Garner, P. Synthesis of tert-Butoxycarbonyl (Boc)-Protected purines. J. Org. Chem. 2000, 65, 7697-7699.
(16) (a) Robins, R. K.; Horner, J. K; Greco, C. V.; Noell, C. W.; Beames Jr, C. G.; Aromaticity in Heterocyclic Systems. I. The Synthesis and Structure of Certain 4,6-Dihydroxyimidazo[4,5-c]pyridines. J. Org. Chem. 1963, 28, 3041-3046. (b) Cook, P. D.; Rousseau, R. J.; Mian, A. M.; Dea, P.; Meyer Jr, R. B.; Robins, R. K Synthesis of 3-Deazaguanine, 3-Deazaguanosine, and 3-deazaguanylic Acid by a Novel ring Closure of Imidazole Precursors. J. Am. Chem. Soc. 1975, 98 (2), 1492-1498.

1. A compound according to the structure I: 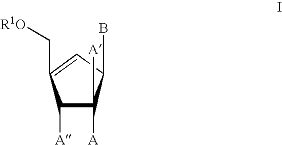
Where B is 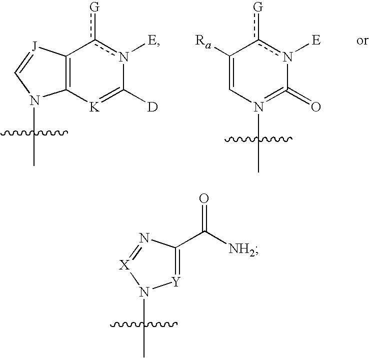
A is H, OR² or halogen (F, Cl, Br, I, preferably F or Br, more preferably F);
A′ is H, OR² or halogen (F, Cl, Br, I, preferably F or Br, more preferably F);
A″ is H or OR¹,
with the proviso that when A′ is OR², A is H; and when A is OR², A′ is H;
X is C—R³ or N;
Y is C—R³ or N; preferably X or Y is N and X and Y are not both simultaneously N;
R³ is H or C₁-C₃ alkyl;
D is H or NHR²;
E is absent (when G is NHR²) or H;
G is O or NHR²;
J is N or C—R⁴;
K is N or C—H;
R⁴ is H, halogen (F, Cl, Br, I), CN, —C(═O)NH₂, NH₂, NO₂, —C═C—H (cis or trans) or —C≡C—H;
Rₐ is H or CH₃;
Each R¹ is independently H, an acyl group, a C₁-C₂₀ alkyl or ether group, a phosphate, diphosphate, triphosphate, phosphodiester group;
Each R² is independently H, an acyl group, a C₁-C₂₀ alkyl or ether group; and
pharmaceutically acceptable salts, solvates or polymorphs thereof.

2. The compound according to claim 1 wherein R¹ and R² are both H.

3. The compound according to claim 1 wherein A and A″ are OH and A′ is H.

4. The compound according to claim 1 wherein A or A′ is halogen.

5. The compound according to claim 1 wherein A″ is H.

6. The compound according to claim 1 wherein J is CR⁴.

7. The compound according to claim 1 wherein G is NHR².

8. The compound according to claim 1 wherein K is N.

9. The compound according to claim 1 wherein K is CH.

10. The compound according to claim 1 wherein J is N, K is CH and G is O or NHR².

11. The compound according to claim 1 where D is NHR².

12. The compound according to claim 1 wherein B is

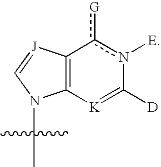

13. The compound according to claim 11 wherein K is N.

14. The compound according to claim 11 wherein K is CH.

15. The compound according to claim 11 wherein J is C—R⁴.

16. The compound according to claim 12 wherein G is O, E is H and D is NHR².

17. The compound according to claim 12 wherein G is NHR², E is non-existent and D is H.

18. The compound according to claim 1, wherein R⁴ is an acetylene group.

19. The compound according to claim 1 wherein said compound is

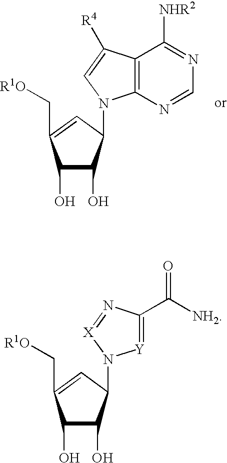

20. The compound according to claim 1 wherein R² is H and R¹ is an acyl group.

21. The compound according to claim 1 wherein X and Y are both CH.

22. The compound according to claim 1 wherein X or Y is N.

23. The compound according to claim 1 which is

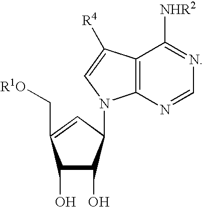

24. The compound according to claim 23 wherein R¹ and R² are both H.

25. A compound according to claim 1 wherein B is:

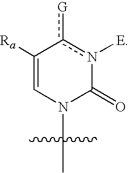

26. The compound according to claim 25 wherein G is O and E is H.

27. The compound according to claim 25 wherein Rₐ is H.

28. The compound according to claim 26 wherein Rₐ is CH₃.

29. The compound according to claim 25 wherein G is NHR², E is non-existent and Rₐ is H.

30. The compound according to claim 25 wherein R¹ and R² are both H.

31. A compound according to the structure: 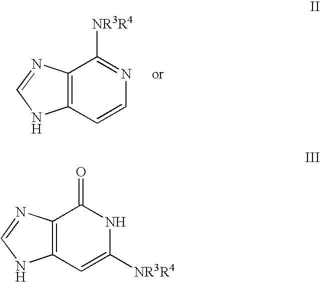
Where R³ and R⁴ are the same or different and are independently H, a COR^{a} group or a COOR^{b} group (preferably R³ and R⁴ are identical), or when R³ and R⁴ are both COR^{a} groups, R³ and R⁴ together with the nitrogen to which they are attached may form a single or multi-ring system having two keto groups alpha to the nitrogen in the single or multi-ring system, with the proviso that both R³ and R⁴ are not simultaneously H;
Each R^{a} is the same or different and is independently a C₁-C₂₅ optionally substituted hydrocarbyl group (preferably each R^{a} is identical);
Each R^{b} is the same or different and is independently a C₁-C₂₅ optionally substituted hydrocarbyl group (preferably each R^{b} is identical); and
salts, solvates and polymorphs thereof.

32-33. (canceled)

34. A pharmaceutical composition comprising an effective amount of a compound according to claim 1 optionally in combination with a pharmaceutically acceptable additive, carrier or excipient.

35. A method of treating a viral infection whose causative agent is a virus selected from the group consisting of an Orthopox virus infection, severe acute respiratory syndrome virus-associated coronavirus (SARS virus), measles virus, human cytomegalovirus (HCMV), hepatitis B virus (HBV), hepatitis C virus (HCV), vaccinia virus, Herpes Simplex virus I (HSV-1), Herpes Simplex virus II (HSV-2), Varicella-Zoster virus (VZV), yellow fever virus, dengue virus, tacaribe virus, Rhinovirus (common cold), adenovirus, influenza A (flu A), influenza B (flu B), respiratory syncytial virus (RSV), parainfluenza virus (PIV), in a patient, comprising administering to said patient an effective amount of a compound or composition according to claim 1 to said patient.

36-39. (canceled)

40. The method according to claim 35 wherein said virus is a drug resistant virus.

41. A method of reducing the likelihood of a viral infection in patient at risk for such an infection, said infection being caused by a virus selected from the group consisting of an Orthopox virus infection, severe acute respiratory syndrome virus-associated coronavirus (SARS virus), measles virus, human cytomegalovirus (HCMV), hepatitis B virus (HBV), hepatitis C virus (HCV), vaccinia virus, Herpes Simplex virus I (HSV-1), Herpes Simplex virus II (HSV-2), Varicella-Zoster virus (VZV), yellow fever virus, dengue virus, tacaribe virus, Rhinovirus (common cold), adenovirus, influenza A (flu A), influenza B (flu B), respiratory syncytial virus (RSV), parainfluenza virus (PIV), comprising administering to said patient at risk an effective amount of a compound or composition according to claim 1 to said patient.

42. A method of synthesizing 3-deazaadenine comprising reacting a compound according to the structure: 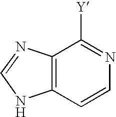
where Y′ is Cl, Br, or I, preferably Cl
With an azide salt (preferably sodium or lithium azide) in the presence of ionic liquid to produce a tricyclic tetrazole compound according to the structure 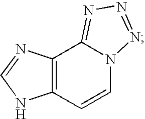
and subjecting said tricyclic tetrazole compound to hydrogenation conditions to convert the azide group to an amino group to produce the compound

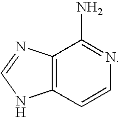

43. (canceled)
